SEQUENCE LISTING (35 sequences)

SEQ ID NO: 1 - protein, 16 aa, Artificial Sequence
  CHDSos-1 Mimic of Sos alpha H
FIGRLCTEILKLREGN

SEQ ID NO: 2 - protein, 16 aa, Artificial Sequence
  CHDSos-1 Mimic of Sos alpha I
LAWRLRELERELARLC

SEQ ID NO: 3 - protein, 16 aa, Artificial Sequence
  CHDSos-2 Mimic of Sos alpha H
WIGRLCTEILRLRNGN

SEQ ID NO: 4 - protein, 16 aa, Artificial Sequence
  CHDSos-2 Mimic of Sos alpha I
LAWRLRELERELARLC

SEQ ID NO: 5 - protein, 16 aa, Artificial Sequence
  CHDSos-3 Mimic of Sos alpha H
AIGRLCTEILRLRNGA

SEQ ID NO: 6 - protein, 16 aa, Artificial Sequence
  CHDSos-3 Mimic of Sos alpha I
LAWRLRELERELARLC

SEQ ID NO: 7 - protein, 16 aa, Artificial Sequence
  CHDSos-4 Mimic of Sos alpha H
WIGRLCTEILRLRNGN

SEQ ID NO: 8 - protein, 16 aa, Artificial Sequence
  CHDSos-4 Mimic of Sos alpha I
LAWALRELERELARLC

SEQ ID NO: 9 - protein, 16 aa, Artificial Sequence
  CHDSos-5 Mimic of Sos alpha H
  MISC_FEATURE (10)..(10): Xaa at position 10 is homoarginine
WIGRLCTEIXRLRNGN

SEQ ID NO: 10 - protein, 16 aa, Artificial Sequence
  CHDSos-5 Mimic of Sos alpha I
LAWRLRELERELARLC

SEQ ID NO: 11 - protein, 16 aa, Artificial Sequence
  CHDSos-6 Mimic of Sos alpha H
WIGRLCTEIRRLRNGN

SEQ ID NO: 12 - protein, 16 aa, Artificial Sequence
  CHDSos-6 Mimic of Sos alpha I
LAWRLRELERELARLC

SEQ ID NO: 13 - protein, 16 aa, Artificial Sequence
  CHDSos-2 alpha H Mimic of Sos alpha H
WIGRLCTEILRLRNGN

SEQ ID NO: 14 - protein, 16 aa, Artificial Sequence
  CHDSos-2 alpha I Mimic of Sos alpha I
LAWRLRELERELARLC

SEQ ID NO: 15 - protein, 16 aa, Artificial Sequence
  FITC-CHDSos-1 Mimic of Sos alpha H
FIGRLCTEILKLREGN

SEQ ID NO: 16 - protein, 18 aa, Artificial Sequence
  FITC-CHDSos-1 Mimic of Sos alpha I
  MISC_FEATURE (2)..(2): Xaa at position 2 is L-beta-alanine
AXLAWRLRELERELARLC

SEQ ID NO: 17 - protein, 16 aa, Artificial Sequence
  FITC-CHDSos-2 Mimic of Sos alpha H
WIGRLCTEILRLRNGN

SEQ ID NO: 18 - protein, 18 aa, Artificial Sequence
  FITC-CHDSos-2 Mimic of Sos alpha I
  MISC_FEATURE (2)..(2): Xaa at position 2 is L-beta-alanine
AXLAWRLRELERELARLC

SEQ ID NO: 19 - protein, 16 aa, Artificial Sequence
  FITC-CHDSos-3 Mimic of Sos alpha H
AIGRLCTEILRLRNGA

SEQ ID NO: 20 - protein, 18 aa, Artificial Sequence
  FITC-CHDSos-3 Mimic of Sos alpha I
  MISC_FEATURE (2)..(2): Xaa at position 2 is L-beta-alanine
AXLAWRLRELERELARLC

SEQ ID NO: 21 - protein, 16 aa, Artificial Sequence
  FITC-CHDSos-4 Mimic of Sos alpha H
WIGRLCTEILRLRNGN

SEQ ID NO: 22 - protein, 18 aa, Artificial Sequence
  FITC-CHDSos-4 Mimic of Sos alpha I
  MISC_FEATURE (2)..(2): Xaa at position 2 is L-beta-alanine
AXLAWALRELERELARLC

SEQ ID NO: 23 - protein, 16 aa, Artificial Sequence
  FITC-CHDSos-5 Mimic of Sos alpha H
  MISC_FEATURE (10)..(10): Xaa at position 10 is homoarginine
WIGRLCTEIXRLRNGN

SEQ ID NO: 24 - protein, 18 aa, Artificial Sequence
  FITC-CHDSos-5 Mimic of Sos alpha I
  MISC_FEATURE (2)..(2): Xaa at position 2 is L-beta-alanine
AXLAWRLRELERELARLC

SEQ ID NO: 25 - protein, 16 aa, Artificial Sequence
  FITC-CHDSos-6 Mimic of Sos alpha H
WIGRLCTEIRRLRNGN

SEQ ID NO: 26 - protein, 18 aa, Artificial Sequence
  FITC-CHDSos-6 Mimic of Sos alpha I
  MISC_FEATURE (2)..(2): Xaa at position 2 is L-beta-alanine
AXLAWRLRELERELARLC

SEQ ID NO: 27 - protein, 16 aa, Artificial Sequence
  DZ1-CHDSos-5 Mimic of Sos alpha H
  MISC_FEATURE (10)..(10): Xaa at position 10 is homoarginine
WIGRLCTEIXRLRNGN

SEQ ID NO: 28 - protein, 17 aa, Artificial Sequence
  DZ1-CHDSos-5 Mimic of Sos alpha I
GLAWRLRELERELARLC

SEQ ID NO: 29 - protein, 17 aa, Artificial Sequence
  DZ2-CHDSos-5 Mimic of Sos alpha H
  MISC_FEATURE (11)..(11): Xaa at position 11 is diazirine photocrosslinker
WIGRLCTEIKXRLRNGN

SEQ ID NO: 30 - protein, 16 aa, Artificial Sequence
  DZ2-CHDSos-5 Mimic of Sos alpha I
LAWRLRELERELARLC

SEQ ID NO: 31 - protein, 29 aa, Artificial Sequence
  ER Transport Peptide Signal Sequence
MMSFVSLLLVGILFYATEAEQLTKCEVFQ

SEQ ID NO: 32 - protein, 4 aa, Artificial Sequence
  ER Retention Signal Sequence
KEDL

SEQ ID NO: 33 - protein, 8 aa, Artificial Sequence
  Nuclear Transport Peptide Sequence
PPKKKRKV

SEQ ID NO: 34 - protein, 25 aa, Artificial Sequence
  Mitochondria Targeting Peptide Sequence
MLSLRQSIRFFKPATRTLCSSRYLL

SEQ ID NO: 35 - protein, 15 aa, Artificial Sequence
  Sos alphaH Helix (929-944)
FFGIYLTNILKTEEG

(19) United States
(12) Patent Application Publication
(10) Pub. No.: US 2024/0124539 A1
(43) Pub. Date: Apr. 18, 2024

(54) CROSSLINKED HELIX DIMER MIMICS OF SOS AND METHODS OF USING SAME

(71) Applicant: NEW YORK UNIVERSITY, New York, NY (US)

(72) Inventors: Paramjit S. ARORA, Cold Spring Harbor, NY (US); Seong Ho HONG, Brooklyn, NY (US); Daniel YOO, Brooklyn, NY (US)

(21) Appl. No.: 18/259,637

(22) PCT Filed: Dec. 23, 2021
(86) PCT No.: PCT/US2021/065055

§ 371 Date: Jun. 28, 2023

Related U.S. Application Data
(60) Provisional application No. 63/131,103, filed on Dec. 28, 2020.

(51) Int. Cl. C07K 14/47

(52) U.S. Cl. CPC C07K 14/4705

(57) ABSTRACT

This invention relates to macrostructures (and pharmaceutical formulations containing them) that include an antiparallel coiled-coil. wherein the antiparallel coiled-coil comprises a first coil of Formula I and a second coil of Formula II: T₁-g₀-a₁-b₁-c₁-d₁-e₁-f₁-g₁-a₂-b₂-c₂-d₂-e₂-f₂-g₂-a₃-b₃-c₃-d₃-e₃-f₃-T₂ (I) T₃-f₀-g′₀-a′₁-b′₁-c′₁-d′₁-e′₁-f′₁-g′₁-a′₂-b′₂-c′₂-d′₂-e′₂-f′₂-g′₂-a′₃-b′₃-c′₃-d′₃-e′₃-T₄ (II), as described in the present application. Methods of using these macrostructures are also disclosed.

This application claims the benefit of U.S. Provisional Patent Application Ser. No. 63/131,103, filed Dec. 28, 2020, the contents of which are incorporated herein by reference in their entirety.

This invention was made with government support under grant number R35GM130333 awarded by the National Institutes of Health. The government has certain rights in the invention.

The instant application contains a Sequence Listing which has been submitted electronically in ASCII format and is hereby incorporated by reference in its entirety. Said ASCII copy, created on Dec. 17, 2021, is named 147462.002271_ST25.txt and is 18,005 bytes in size.

### FIELD OF THE INVENTION

This invention is directed to stabilized coiled coils that mimic the Sos αH/αI coiled coil and methods of using such mimics.

### BACKGROUND OF THE INVENTION

Aberrant Ras signaling is linked to a wide spectrum of hyperproliferative diseases, and components of the signaling pathway, including Ras, have been the subject of intense and ongoing drug discovery efforts. The cellular activity of Ras is modulated by its association with the guanine nucleotide exchange factor Son of Sevenless (Sos), and the high-resolution crystal structure of the Ras-Sos complex provides a basis for the rational design of orthosteric Ras ligands.

Oncogenic Ras isoforms are the subject of intense study due to the difficulty in targeting these biomedically important yet “undruggable” proteins. Recent success in covalent targeting of a Ras mutant illustrates potential avenues for ligand design; however, many mutant Ras forms do not feature nucleophilic residues suggesting that strategies for noncovalent engagement of Ras are required.

The Ras-specific guanine nucleotide exchange factor Sos mediates the conversion of Ras from its inactive GDP-bound form to the active GTP-bound state (P. A. Konstantinopoulos, M. V. Karamouzis, A. G. Papavassiliou, Post-translational modifications and regulation of the RAS superfamily of GTPases as anticancer targets. Nat. Rev. Drug Discov. 6, 541-555 (2007)). Sos catalyzes nucleotide exchange via insertion of a critical helical segment (αH) between the conformationally dynamic Switch I and II regions that flank the Ras nucleotide binding pocket leading to disruption of water-mediated and direct interactions between the protein and the cofactor (see FIG. 1A) (P. A. Boriack-Sjodin, S. M. Margarit, D. Bar-Sagi, J. Kuriyan, The structural basis of the activation of Ras by Sos. Nature 394, 337-343 (1998)). Given the biomedical importance of the conformationally dynamic Sos-binding interface of Ras, several rational design and screening strategies have been attempted to isolate ligands for this interface (A. R. Moore, S. C. Rosenberg, F. McCormick, S. Malek, RAS-targeted therapies: is the undruggable drugged? Nature Reviews Drug Discovery 19, 533-552 (2020) .; H. Chen, J. B. Smaill, T. Liu, K. Ding, X. Lu, Small-Molecule Inhibitors Directly Targeting KRAS as Anticancer Therapeutics. Journal of Medicinal Chemistry 63, 14404-14424 (2020)). Recent efforts to engage the Ras G12C (J. Canon et al., The clinical KRAS(G12C) inhibitor AMG 510 drives anti-tumour immunity. Nature 575, 217-223 (2019); J. M. Ostrem, U. Peters, M. L. Sos, J. A. Wells, K. M. Shokat, K-Ras(G12C) inhibitors allosterically control GTP affinity and effector interactions. Nature 503, 548-551 (2013)) and the G12D isoforms (Z. Zhang et al., GTP-State-Selective Cyclic Peptide Ligands of K-Ras(G12D) Block Its Interaction with Raf. ACS Central Science 6, 1753-1761 (2020); K. Sakamoto, T. Masutani, T. Hirokawa, Generation of KS-58 as the first K-Ras(G12D)-inhibitory peptide presenting anti-cancer activity in vivo. Scientific Reports 10, 21671 (2020)) suggest that targeted screens may afford small molecules and peptide macrocycles as potential leads.

The structure of the Ras-Sos complex provides a basis for the rational design of Sos helix mimics that engage the Ras switch regions. Indeed, a conformationally stabilized α-helix mimic to target the Ras-Sos protein-protein interaction (PPI) has previously been developed (D. Y. Yoo, A. D. Hauser, S. T. Joy, D. Bar-Sagi, P. S. Arora, Covalent Targeting of Ras G12C by Rationally Designed Peptidomimetics. ACS Chem. Biol. 15, 1604-1612 (2020); A. Patgiri, K. K. Yadav, P. S. Arora, D. Bar-Sagi, An orthosteric inhibitor of the Ras-Sos interaction. Nat. Chem. Biol. 7, 585-587 (2011)). The stabilized a-helix was shown to bind Ras at the orthosteric binding site and inhibit Sos-mediated nucleotide exchange, Ras activation, and phosphorylation of the downstream effector protein ERK (B. N. Kholodenko, J. F. Hancock, W. Kolch, Signalling ballet in space and time. Nature Reviews Molecular Cell Biology 11, 414-426 (2010)), a well-characterized kinase implicated in cell proliferation and differentiation. However, this compound preferred to bind Ras in its nucleotide-free form, suggesting that a single Sos helix is likely insufficient to properly engage the dynamic Ras interface in its nucleotide-bound form. While wild-type Ras toggles between its two nucleotide-bound forms (FIG. 1A), the oncogenic forms of Ras remain activated in their GTP-bound states. Therefore, a compound that preferentially engages the nucleotide-free form of Ras may have limited biological utility.

Prior results with the Sos helix mimic encapsulate a critical challenge in developing minimal protein secondary structure mimics. Although mimics of protein secondary structures have proven to be a potent class of protein-protein interaction (PPI) inhibitors (Y. S. Chang et al., Stapled alpha-helical peptide drug development: a potent dual inhibitor of MDM2 and MDMX for p53-dependent cancer therapy. Proc. Natl. Acad. Sci. USA 110, E3445-3454 (2013); S. Kushal et al., Protein domain mimetics as in vivo modulators of hypoxia-inducible factor signaling. Proc. Natl. Acad. Sci. USA 110, 15602-15607 (2013); S. Liu, R. W. Cheloha, T. Watanabe, T. J. Gardella, S. H. Gellman, Receptor selectivity from minimal backbone modification of a polypeptide agonist. Proc. Natl. Acad. Sci. USA 115, 12383-12388 (2018); M. Pelay-Gimeno, A. Glas, O. Koch, T. N. Grossmann, Structure-Based Design of Inhibitors of Protein-Protein Interactions: Mimicking Peptide Binding Epitopes. Angew. Chem. Int. Ed. 54, 8896-8927 (2015)), many protein interfaces feature binding epitope complexity beyond what can be captured by simple secondary structure mimics (W. S. Horne, T. N. Grossmann, Proteomimetics as protein-inspired scaffolds with defined tertiary folding patterns. Nat. Chem. 12, 331-337 (2020); H. Adihou et al., A protein tertiary structure mimetic modulator of the Hippo signalling pathway. Nat. Commun. 11, 5425 (2020); J. Sadek et al., Modulation of virus-induced NF-kappaB signaling by NEMO coiled coil mimics. Nat Commun 11, 1786 (2020); A. M. Watkins, M. G. Wuo, P. S. Arora, Protein-Protein Interactions Mediated by Helical Tertiary Structure Motifs. J. Am. Chem. Soc. 137, 11622-11630 (2015)).

Thus there exists an unmet need for developing minimal Sos protein secondary structure mimics as potential therapeutic targets.

### SUMMARY OF THE INVENTION

Various non-limiting aspects and embodiments of the invention are described below.

In one aspect, the present invention provides a macrostructure comprising an antiparallel coiled-coil, wherein the antiparallel coiled-coil comprises: a first coil of Formula I and a second coil of Formula II:

T₁-g₀-a₁-b₁-c₁-d₁-e₁-f₁-g₁-a₂-b₂-c₂-d₂-e₂-f₂-g₂-a₃-b₃-c₃-d₃-e₃-f₃-T₂ (I)

T₃-f′₀-g′₀-a′₁-b′₁-c′₁-d′₁-e′₁-f′₁-g′₁-a′₂-b′₂-c′₂-d′₂-e′₂-f′₂-g′₂-a′₃-b′₃-c′₃-d′₃-e′₃-T₄ (II),
wherein: each a₁₋₃, b₁₋₃, c₁₋₃, d₁₋₃, e₁₋₃, f₁₋₃, g₀₋₂, a′₁₋₃, b′₁₋₃, c′₁₋₃, d′₁₋₃, e′₁₋₃, f′₀₋₂, and g′₀₋₂ is independently absent or a residue selected from the group consisting of modified or unmodified amino acid residues and analogues thereof;
one or more of the following residue pairs are covalently bound by a linker: g₀-g′₂, g₁-g′₁, g₂-g′₀, a₁-d′₃, a₂-d′₂, a₃-d′₁, d₁-a′₃, d₂-a′₂, d₃-a′₁, e₁-e′₃, e₂-e′₂, and e₃-e′₁;
each T₁ and T₃ is independently a point of attachment from a terminal nitrogen to one or more (preferably one or two) moieties, wherein each moiety is independently H, -PG₁, —C(O)R, —C(O)NR₂, —C(O)NH₂, —R, —C(O)OR, an amino acid or analogue thereof, a peptide, a targeting moiety, or a tag, where PG₁ is an amine protecting group and each R is independently hydrogen, an alkyl, an alkenyl, an alkynyl, a cycloalkyl, an aryl, a heteroaryl, a heterocyclyl, an arylalkyl, a peptide, a targeting moiety, or a tag; and
each T₂ and T₄ is independently a point of attachment from a terminal carbonyl to H, —OPG₂, —NPG₂, —OR, —OH,—NR₂, —NH₂, —N(R)C(O)C₁₋₆ alkyl, —N(H)C(O)C₁₋₆ alkyl, an amino acid or analogue thereof, a peptide, a targeting moiety, or a tag, where PG₂ is a carboxylic acid protecting group and each R is independently hydrogen, an alkyl, an alkenyl, an alkynyl, a cycloalkyl, an aryl, a heteroaryl, a heterocyclyl, an arylalkyl, a peptide, a targeting moiety, or a tag; and wherein: the first coil comprises at least ten contiguous residues, wherein the at least ten contiguous residues have the formula X₁-X₂-X₃-X₄-X₅-X₆-X₇-X₈-X₉-X₁₀-X₁₁-X₁₂-X₁₃-X₁₄-X₁₅-X₁₆;
the second coil comprises at least ten contiguous residues, wherein the at least ten contiguous residues have the formula X′₁-X′₂-X′₃-X′₄-X′₅-X′₆-X′₇-X′₈-X′₉-X′₁₀-X′₁₁-X′₁₂-X′₁₃-X′₁₄- X′₁₅-X′_{16;}
and wherein each residue is selected from the groups indicated below (superscript letters indicate each residue's location within Formula I and Formula II; residues in the a, a′, d, d′, e, e′, g, and g′ positions can optionally be modified to facilitate attachment of a linker or replaced with a linker; underlined residues are particularly preferred)

| First Coil | | | Second Coil | | |
|---|---|---|---|---|---|
| | | Preferred | | | Preferred |
| Residue | Group | Residue(s) | Residue | Group | Residue(s) |
| ^{g}X₁ | Any residue | Phe, Trp | | | |
| ^{a}X₂ | Any | Cys, HCys, Leu, | ^{d}X′₁ | Any | Cys, HCys, Leu, |
| | hydrophobic | Ile, allylleucine, | | hydrophobic | Ile, allylleucine, |
| | residue | Val, allylglycine, | | residue | Val, allylglycine, |
| | | Thr, | | | Thr, |
| | | selenocysteine, | | | selenocysteine, |
| | | hexafluoroleucine, | | | hexafluoroleucine, |
| | | hexafluorovaline | | | hexafluorovaline |
| | | (or analogue of | | | (or analogue of |
| | | any of the | | | any of the |
| | | preceding | | | preceding |
| | | residues) | | | residues) |
| ^{b}X₃ | Any residue | Gly | ^{e}X′₂ | Any residue | Ala |
| ^{c}X₄ | Any residue | Arg | ^{f}X′₃ | Any residue | Trp |
| ^{d}X₅ | Any | Cys, HCys, Leu, | ^{g}X′₄ | Any residue | Arg |
| | hydrophobic | Ile, allylleucine, | | | |
| | residue | Val, allylglycine, | | | |
| | | Thr, | | | |
| | | selenocysteine, | | | |
| | | hexafluoroleucine, | | | |
| | | hexafluorovaline | | | |
| | | (or analogue of | | | |
| | | any of the | | | |
| | | preceding | | | |
| | | residues) | | | |
| ^{e}X₆ | Any residue | Cys | ^{a}X′₅ | Any | Cys, HCys, Leu, |
| | | | | hydrophobic | Ile, allylleucine, |
| | | | | residue | Val, allylglycine, |
| | | | | | Thr, |
| | | | | | selenocysteine, |
| | | | | | hexafluoroleucine, |
| | | | | | hexafluorovaline |
| | | | | | (or analogue of |
| | | | | | any of the |
| | | | | | preceding |
| | | | | | residues) |
| ^{f}X₇ | Any residue | Thr | ^{b}X′₆ | Any residue | Arg |
| ^{g}X₈ | Any residue | Glu, Carboxylic | ^{c}X′₇ | Any residue | Glu |
| | | Acid Isostere | | | |
| ^{a}X₉ | Any | Cys, HCys, Leu, | ^{d}X′₈ | Any | Cys, HCys, Leu, |
| | hydrophobic | Ile, allylleucine, | | hydrophobic | Ile, allylleucine, |
| | residue | Val, allylglycine, | | residue | Val, allylglycine, |
| | | Thr, | | | Thr, |
| | | selenocysteine, | | | selenocysteine, |
| | | hexafluoroleucine, | | | hexafluoroleucine, |
| | | hexafluorovaline | | | hexafluorovaline |
| | | (or analogue of | | | (or analogue of |
| | | any of the | | | any of the |
| | | preceding | | | preceding |
| | | residues) | | | residues) |
| ^{b}X₁₀ | Any residue | Leu, L- | ^{e}X′₉ | Any residue | Glu |
| | | homoarginine | | | |
| ^{c}X₁₁ | Any residue | Lys, Arg | ^{f}X′₁₀ | Any residue | Arg |
| ^{d}X₁₂ | any | Cys, HCys, Leu, | ^{g}X′₁₁ | Any residue | Glu |
| | hydrophobic | Ile, allylleucine, | | | |
| | residue | Val, allylglycine, | | | |
| | | Thr, | | | |
| | | selenocysteine, | | | |
| | | hexafluoroleucine, | | | |
| | | hexafluorovaline | | | |
| | | (or analogue of | | | |
| | | any of the | | | |
| | | preceding | | | |
| | | residues) | | | |
| ^{e}X₁₃ | Any residue | Arg | ^{a}X′₁₂ | Any | Cys, HCys, Leu, |
| | | | | hydrophobic | Ile, allylleucine, |
| | | | | residue | Val, allylglycine, |
| | | | | | Thr, |
| | | | | | selenocysteine, |
| | | | | | hexafluoroleucine, |
| | | | | | hexafluorovaline |
| | | | | | (or analogue of |
| | | | | | any of the |
| | | | | | preceding |
| | | | | | residues) |
| ^{f}X₁₄ | Any residue | Glu, Asn, Amide | ^{b}X′ ₁₃ | Any residue | Ala |
| | | isostere, | | | |
| | | Carboxylic acid | | | |
| | | isostere | | | |
| ^{g}X₁₅ | Any residue | Gly | ^{c}X′₁₄ | Any residue | Arg |
| ^{a}X₁₆ | Any residue | Asn, Amide | ^{d}X′₁₅ | Any | Cys, HCys, Leu, |
| | | isostere | | hydrophobic | Ile, allylleucine, |
| | | | | residue | Val, allylglycine, |
| | | | | | Thr, |
| | | | | | selenocysteine, |
| | | | | | hexafluoroleucine, |
| | | | | | hexafluorovaline |
| | | | | | (or analogue of |
| | | | | | any of the |
| | | | | | preceding |
| | | | | | residues) |
| | | | ^{e}X′₁₆ | Any residue | Cys |

In one embodiment, the length of any linker between residue pairs g₀-g′₂, g₁g′₁, g₂-g′₀, e₁-e′₃, e₂-e′₂, and e₃-e′₁ is such that the spatial distance between the Cα positions of each residue in the pair is 10-25 Å; and the length of any linker between residue pairs a₁-d′₃, a₂-d′₂, a₃-d′₁, d₁-a′₃, d₂-a′₂, and d₃-a′₁is such that the spatial distance between the Cα positions of each residue in the pair is 5-15 Å.

In one embodiment, at least g₀, a₁, b₁, c₁, d₁, e₁, f₁, g₁, a₂, b₂, c₂, d₂, e₂, f₂, g₂, and a₃, are present in the first coil and at least d′₁, e′₁, f′₁, g′₁, a′₂, b′₂, c′₂, d′₂, e′₂, f′₂, g′₂, a′₃, b′₃, c′₃, d′₃, and e′₃ are present in the second coil.

In one embodiment, each residue independently has the formula

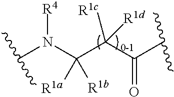

wherein:
R¹, R^{1b}, R^{1c}, and R^{1d} are each independently hydrogen, an amino acid side chain, an alkyl, an alkenyl, an alkynyl, a cycloalkyl, a heterocyclyl, an aryl, a heteroaryl, or an arylalkyl, wherein each amino acid side chain, alkyl, alkenyl, alkynyl, cycloalkyl, heterocyclyl, aryl, heteroaryl, and arylalkyl can be optionally substituted with H, an alkyl, an alkenyl, an alkynyl, an azide, —OR⁵, or —SR⁵; and wherein when a linker covalently binds to a residue, the linker is attached to or replaces one of R¹, R^{1b}, R^{1c}, and R^{1d}, each R⁴ is independently hydrogen, an alkyl, an alkenyl, an alkynyl, a cycloalkyl, a heterocyclyl, an aryl, a heteroaryl, or an arylalkyl; and each R⁵ is independently selected from the group consisting of H, -PG (where PG is a protecting group), an alkyl, an alkenyl, an alkynyl, a cycloalkyl, an aryl, a heteroaryl, a heterocyclyl, and an arylalkyl.

In one embodiment, a linker between at least one of residue pairs g₀-g′₂, g₁-g′₁, g₂-g′₀, e₁-e′₃, e₂-e′₂, and e₃-e′₁ is present.

In one embodiment, a linker between at least one of residue pairs g₀-g′₂, g₁-g′₁, g₂-g′₀, e₁-e′₃, e₂-e′₂, and e₃-e′₁ has the formula -Zₙ-, wherein n is a number from 1 to 25 and each Z is independently selected at each occurrence thereof from the group consisting of alkylene, alkenylene, arylene, heteroarylene, triazole-diyl, thiazole-diyl, oxazole-diyl, ethers, amides, esters, maleimides, thioethers, O, S, and Se.

In one embodiment, a linker between at least one of residue pairs g₀-g′₂, g₁-g′₁, g₂-g′₀, e₁-e′₃, e₂-e′₂, and e₃-e′₁ has a formula selected from (a) the group consisting of:

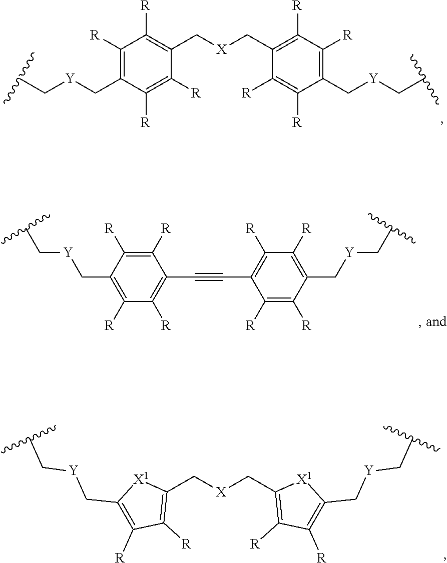

(b) the group consisting of:

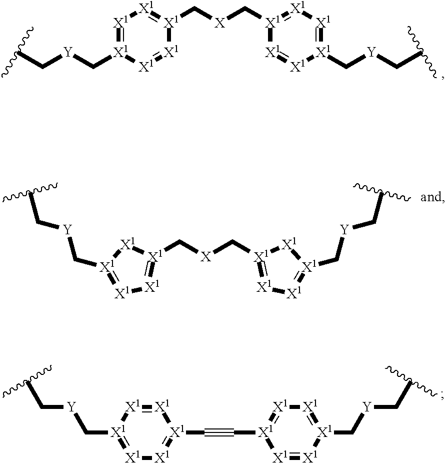

wherein:
X in group (a) and group (b) is O, S, CR₂, NR, or P (preferably O, S, CH₂ or NR);
each X¹ in group (a) is independently O, S, NH, or NR;
each X¹ in group (b) is independently O, S, C, CR, N, NH, or NR;
each R in group (a) and group (b) is independently H, alkyl, or aryl; and
each Y in group (a) and group (b) is S.

In one embodiment, a linker between at least one of residue pairs g₀-g′₂, g₁-g′₁, g₂-g′₀, e₁-e′₃, e₂-e′₂, and e₃-e′₁ is selected from the group consisting of

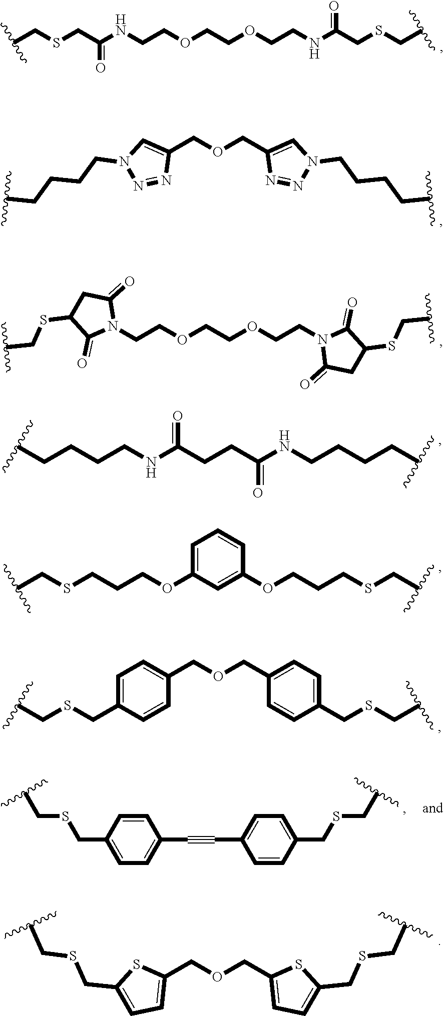

In one embodiment, a linker between at least one of residue pairs a₁-d′₃, a₂-d′₂, a₃-d′₁, d₁-a′₃, d₂-a′₂, and d₃-a′₁ is present.

In one embodiment, a linker between at least one of residue pairs a₁-d′₃, a₂-d′₂, a₃-d′₁, d₁-a′₃, d₂-a′₂, and d₃-a′₁ is selected from the group consisting of disulfides, diselenides, C₁₋₈ alkylene, C₂₋₈ alkenylene, arylene, heteroarylene, triazole-diyl, and thiazole-diyl.

In one embodiment, a linker between at least one of residue pairs a₁-d′₃, a₂-d′₂, a₃-d′₁, d₁-a′₃, d₂-a′₂, and d₃-a′₁ is a disulfide bond from a cysteine or homocysteine residue, a diselenide from a selenocysteine residue, an alkylene from an allylglycine residue, or an arylene linker.

In one embodiment, the antiparallel coiled-coil is of Formula III:
wherein: each dotted line represents, independently, an optional linker and each residue independently has the formula

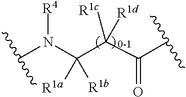

wherein:
R¹, R^{1b}, R^{1c}, and R^{1d} are each independently hydrogen, an amino acid side chain, an alkyl, an alkenyl, an alkynyl, a cycloalkyl, a heterocyclyl, an aryl, a heteroaryl, or an arylalkyl, wherein each amino acid side chain, alkyl, alkenyl, alkynyl, cycloalkyl, heterocyclyl, aryl, heteroaryl, and arylalkyl can be optionally substituted with H, an alkyl, an alkenyl, an alkynyl, an azide, —OR⁵, or —SR⁵; and wherein when a linker covalently binds to a residue, the linker is attached to or replaces one of R¹, R^{1b}, R^{1c}, and R^{1d}, each R⁴ is independently hydrogen, an alkyl, an alkenyl, an alkynyl, a cycloalkyl, a heterocyclyl, an aryl, a heteroaryl, or an arylalkyl; and each R⁵ is independently selected from the group consisting of H, -PG (where PG is a protecting group), an alkyl, an alkenyl, an alkynyl, a cycloalkyl, an aryl, a heteroaryl, a heterocyclyl, and an arylalkyl.

In one embodiment, at least one of the following conditions is met:
(A) in at least one a, a′, d, or d′ residue, (i) one of R^{1a} and R^{1c} is the side chain of a modified or unmodified amino acid selected from the group consisting of cysteine, homocysteine, selenocysteine, leucine, isoleucine, hexafluoroleucine, valine, hexafluorovaline, allylglycine, threonine, and analogues of each of the preceding residues, and (ii) R^{1b}, R^{1d}, and the other of R^{1a} and R^{1c} are each independently hydrogen, a C₁₋₃ alkyl, or a C₂₋₃ alkenyl;
(B) in at least one e, e′, g, or g′ residue, (i) one of R^{1a} and R^{1c} is an amino acid side chain and (ii) R^{1b}, R^{1d}, and the other of R^{1a} and R^{1c} are each independently hydrogen or a C₁₋₃ alkyl.

In one embodiment, the antiparallel coiled-coil has the formula:
wherein each dotted line is independently an optional linker.

In one embodiment, the macrostructure is CHD-1, CHD-2, CHD-3, CHD-4, or CHD-5.

In another aspect, the present invention provides a pharmaceutical composition comprising a macrostructure according to any one of the above embodiments and a pharmaceutically acceptable vehicle.

In yet another aspect, the present invention provides a method of inhibiting Ras signaling in a cell, said method comprising:

contacting the cell with a macrostructure according to any one of the above embodiments

under conditions effective to inhibit Ras signaling in the cell.

In one embodiment, the cell is a mammalian cell.

In one embodiment, the cell expresses a mutated Ras protein.

In one embodiment, said contacting is carried out in a subject.

In yet another embodiment, the present invention provides a method of treating in a

subject a disorder mediated by Ras signaling, said method comprising: administering to the subject a macrostructure according to any one of the above embodiments or a pharmaceutical formulation according to the above embodiment under conditions effective to treat the disorder in the subject.

In yet another embodiment, the present invention provides a method of treating a cellular proliferative disorder, differentiative disorder, and/or neoplastic condition in a subject in need thereof, the method comprising: administering to the subject a macrostructure according to any one of the above embodiments or a pharmaceutical formulation according to the above embodiment under conditions effective to treat the cellular proliferative disorder, differentiative disorder, and/or neoplastic condition in the subject.

In one embodiment, the cellular proliferative disorder, differentiative disorder, and/or neoplastic condition is selected from the group consisting of fibrosarcoma, myosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, gastric cancer, esophageal cancer, rectal cancer, pancreatic cancer, ovarian cancer, prostate cancer, uterine cancer, cancer of the head and neck, skin cancer, brain cancer, squamous cell carcinoma, sebaceous gland carcinoma, papillary carcinoma, papillary adenocarcinoma, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular cancer, small cell lung carcinoma, non-small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma, leukemia, lymphoma, and Kaposi sarcoma; hematopoietic neoplastic disorders; cellular proliferative and/or differentiative disorders of the breast; cellular proliferative and/or differentiative disorders of the lung; cellular proliferative and/or differentiative disorders of the colon; cellular proliferative and/or differentiative disorders of the liver; cellular proliferative and/or differentiative disorders of the ovary; a cancer mediated by a mutated Ras protein; and immunoproliferative disorders.

In one embodiment, the disorder and/or condition is pancreatic ductal adenocarcinoma, rectal adenocarcinoma, plasma cell myeloma, colon adenocarcinoma, bile duct carcinoma, chronic myelomonocytic leukemia, acute myeloid leukemia, melanoma, lung adenocarcinoma, rhabdomyosarcoma, endometrium carcinoma, salivary gland carcinoma, thyroid carcinoma, bladder carcinoma, mouth carcinoma, or ovarian carcinoma.

In one embodiment, the subject is a mammal. In one embodiment, the subject is a primate (e.g., human).

In one embodiment, the disorder or condition is mediated by a mutated Ras protein. In one embodiment, the mutated Ras protein is an H-Ras isoform. In one embodiment, the mutated Ras protein is a K-Ras isoform. In one embodiment, the mutated Ras protein has one or more mutations selected from the group consisting of G12C, G12D, G12S, G12V, G13D, G12A, G12C, G12D, G12R, G12S, G12V, G13A, G13C, G13R, G13S, G13D, Q61E, Q61H, Q61L, Q61K, Q61P, and Q61R.

These and other aspects of the present invention will become apparent to those skilled in the art after a reading of the following detailed description of the invention, including the appended claims.

### BRIEF DESCRIPTION OF THE DRAWINGS

FIG. 1 provides an overview of the Ras activation cycle and design of a Sos-based proteomimetic. The cellular activity of Ras is tightly controlled as part of a balanced feedback loop. Oncogenic mutations shift this balance and increase the cellular concentration of Ras-GTP leading to aberrant downstream signaling. FIG. 1A depicts the molecular model showing complex between Ras (gray ribbon) and its guanine exchange factor Sos. Sos inserts a helical hairpin (helices) into the nucleotide binding pocket of Ras to mediate nucleotide exchange. The Ras nucleotide binding pocket is highlighted. Segments of Sos are not shown to highlight interactions of the helical hairpin with Ras (PDB code INVW). FIG. 2B depicts the molecular models depicting critical Sos helices and the design of a constrained Sos proteomimetic as a Ras inhibitor. GDP =guanosine 5′-diphosphate, GTP=guanosine 5′-triphosphate, GAP=GTPase activating protein, GEF=guanine nucleotide exchange factor.

FIG. 2 depicts the rational design of Sos proteomimetics as Ras ligands. Antiparallel helix wheel diagrams depicting native (top) Sos helical hairpin and (bottom) the optimized constrained helix dimer mimic. FIG. 2A depicts Sos αH and αI helical domains making direct contacts with Sos, with many of the energetically important Ras contacting residues, termed hot spot residues, positioned on the αH helix. FIG. 2B shows designed and synthesized constrained Sos mimics with a hydrophobic interface and non-native residues on both helices to enhance binding interactions with Ras. A dibenzyl ether crosslinker is placed at the ‘e’ position of each helix to enhance conformational stability. The binding affinity of the Sos derivatives for Ras was measured by a fluorescence polarization (FP) assay; the binding affinity of the lead derivative CHC^{Sos}-5 and alanine control CHC^{Sos}-3 was further confirmed by microscale thermophoresis (MST). R^{H}=L-homoarginine.

FIG. 3 shows biophysical characterization, proteolytic stability and the Ras-binding site analysis of CHC^{Sos}-5. FIG. 3A is a circular dichroism spectrum of CHC^{Sos}-5. The CD study was conducted in 50 mM aqueous potassium fluoride buffer (pH 7.5) at 20 μM peptide concentration. FIG. 3B depicts proteolytic stability of CHC^{Sos}-5 in 25% fetal bovine serum analyzed in an HPLC assay as discussed in Example 23. Error bars are mean±SD of biological replicates. FIG. 3C depicts ¹H-¹⁵N HSQC titration spectra of uniformly ¹⁵N-labeled GDP-loaded wild-type H-Ras. Examples of specific Ras residues that shift upon titration with increasing equivalents (1, 2.5 and 5) of CHC^{Sos}-5. FIG. 3D is a bar graph showing mean chemical shift changes observed for the ¹⁵N-labeled H-Ras upon titrations with increasing amounts of CHC^{Sos}-5. FIG. 3E shows the CHC^{Sos}-5 binding site on Ras further confirmed by a proximity-guided protein crosslinking reaction. DZ1-CHC^{Sos}-5 contains a photoactivable diazirine group that reacts with proximal residues on Ras. A fragment with mass corresponding to DZ1-CHC^{Sos}-5 crosslinked to Ras switch I loop was identified. The identified fragment corresponds to the Switch I region and is depicted as a ribbon in the molecular model.

FIG. 4 depicts cellular internalization and efficacy of Sos Proteomimetics modulated with oncogenic Ras mutations. FIG. 4A is live cell fluorescence imaging of Hoechst-stained Ras mutant T24 and H358 cells incubated with fluorescently-labeled CHC^{Sos}-5 or DMSO for 4 hours at 40× (scale bar=5 μm) magnification. FIG. 4B flow cytometry analysis of fluorescently labeled CHC^{Sos}-5 (1 μM) in T24, H358, SW780, BxPC3, and HeLa cells after 1 h treatment. FIG. 4C is fluorescence polarization and microscale thermophoresis analyses were performed to determine the binding affinity of CHC^{Sos}-5 and GDP-loaded H-Ras wild-type and mutant isoforms. FIG. 4D shows the cellular toxicity of CHC^{Sos}-5 analyzed in an MTT cell viability assay. Bar graph shows viability of Ras wild type and mutant cell lines treated with increasing concentrations of CHC^{Sos}-5. The results from the MTT assay were confirmed in the CellTiter-Glo luminescent cell viability assay (Example 26). FIG. 4E is a double y-axis graph showing correlation of CHC^{Sos}-5 cellular uptake and toxicity. Results from cellular uptake studies (left axis) with 1 μM fluorescent analog and MTT cell viability assay (right axis) at 10 μM concentration are shown. FIG. 4F is a representative Western blot showing Erk phosphorylation levels in H358 cells upon treatment with 0, 1, 5, 10 μM CHC^{Sos}-5. FIG. 4G is Western blots showing ERK phosphorylation levels in HeLa and H358 cells upon treatment with CHC^{Sos}-5 or negative control CHC^{Sos}-3. FIG. 4A shows bar graphs comparing ERK phosphorylation in HeLa and H358 cells post-treatment with CHC^{Sos}-3 and CHC^{Sos}-5. Error bars are mean±SD of biological duplicates.

FIG. 5 shows an analysis of Sos Proteomimetic Cellular Interacting Partners by quantitative MS-based proteomics. FIG. 5A is a schematic depicting proximity-driven photocrosslinking of DZ2-CHC^{Sos}-5 with cellular proteins. FIG. 5B is a volcano plot revealing statistically significant (p<0.01) enriched proteins in H358 cells upon treatment with DZ2-CHC^{Sos}-5 (10 μM) for 4 hours. Enriched protein targets that are members of the Ras GTPase superfamily are labeled. FIG. 5C depicts pie charts outlining functional diversity (left) and cellular localization (right) of the enriched protein targets.

FIG. 6 depicts alanine scanning mutagenesis of the Ras (light gray)-Sos (dark grey) complex revealing critical Ras binding residues (spheres). Although, the energetically important binding residues are dispersed over the large Sos surface, the αH helix (dark) contains a cluster of these residues thereby providing a lead helical domain for rational design of Ras ligands.

FIG. 7 shows fluorescence polarization curves for binding of fluorescently-labeled CHDs and GDP-bound wild-type H-Ras. The calculated dissociation constants (K_{d}) for each CHD are listed in the Table. Error bars are mean±SD of triplicates.

FIG. 8 depicts microscale thermophoresis analysis of CHD binding with wild-type and mutant H-Ras labeled with AFDye 647. FIG. 8A is a dose-response curve for interaction between CHC^{Sos}-5 and CHC^{Sos}-3 and wild-type H-Ras-GDP. FIG. 8B is a dose-response curve for interaction between CHC^{Sos}-5 and CHC^{Sos}-3 and G12V H-Ras-GDP. Changes in TRIC signal observed from microscale thermophoresis are plotted as Fnorm vs. Ligand concentrations. Thermographs of wild-type H-Ras-GDP binding to FIG. 8C CHC^{Sos}-5 and FIG. 8D CHC^{Sos}-3. Aggregation was observed with CHC^{Sos}-3 at concentration higher than 3 μM and interrupted binding isotherm. Thermographs of G12V H-Ras-GDP binding to FIG. 8E CHC^{Sos}-5 and FIG. 8F CHC^{Sos}-3. Cold region is set to 0 sec and hot region at 3 sec are used. Error bars are mean±SD from triplicates.

FIG. 9 shows circular dichroism spectra of crosslinked and linear peptides. CD spectra of the individual unlinked αH and αI peptides, equimolar mixture of the αH and αI peptides, and the CHC^{Sos}-2. CD experiments were conducted in 50 mM aqueous potassium fluoride buffer (pH 7.5) with 20 μM peptide concentration.

FIG. 10 shows titration HSQC NMR spectroscopy revealing CHC^{Sos}-5/Ras binding site. ¹H-¹⁵N HSQC overlaid spectra of H-Ras alone, H-Ras:CHC^{Sos}-5 (1:2.5), and H-Ras:CHC^{Sos}-5 (1:5).

FIG. 11 shows chemical crosslinking of diazirine-modified CHC^{Sos}-5 supports Ras binding site. FIG. 11A is a gel shift assay with H-Ras incubated with and without DZ1-CHC^{Sos}-5. FIG. 11B shows MALDI-TOF spectra displaying identified fragment masses of trypsin-digested DZ1-CHC^{Sos}-5 crosslinked to the Ras switch I loop. Corresponding labeled mass was not observed from the unlabeled Ras sample.

FIG. 12 depicts fluorescence microscopy and flow cytometry studies to analyze cellular uptake of CHDs. FIG. 12A shows live cell fluorescence imaging of Hoechst-stained Ras mutant T24, H358, and BxPC3 cells incubated with fluorescently-labeled peptides and DMSO (negative control) for 4 hours at 40× (scale bar=5 μm) magnification. Cells were exposed to 500 nM of CHDs and Tat. FIG. 12B shows median fluorescence intensities in T24 and H358 cells post-treatment with 1 μM CHC^{Sos}-2, CHC^{Sos}-3, and CHC^{Sos}-5 and Tat for 1 hour. FIG. 12C and FIG. 12D show flow cytometry analysis of fluorescently labeled CHC^{Sos}-5 (1 μM) in indicated cell lines after 1 h treatment. Error bars are mean±SD of biological duplicates.

FIG. 13 shows MTT and CellTiter-Glo assays to determine cell viability in the presence of CHDs. MTT cell viability in indicated Ras mutant and control cell lines treated with increasing concentrations of FIG. 13A CHC^{Sos}-2 and FIG. 13B CHC^{Sos}-3. The cell viability studies were performed in the presence of serum. FIG. 13C shows cell viability assessed by CellTiter-Glo assay in H358 and HeLa cells treated with increasing concentrations of CHC^{Sos}-5. Error bars are mean±SD of biological triplicates.

FIG. 14 depicts representative Western blots to probe the effect of CHC^{Sos}-5 on

the levels of active/GTP-bound Ras. Ras-GTP levels were determined by immunoblotting with Ras-GTP-specific antibody after treatment of H358 cells with 10 μM CHC^{Sos}-5 for 6 hours.

FIG. 15 depicts representative Western blots to analyze ERK phosphorylation. Suppression of ERK phosphorylation by CHDs was probed in H358 and HeLa cells after treatment with (A) CHC^{Sos}-5 and (B) CHC^{Sos}-3 (DMSO, 1, 5, 10 μM). Error bars are mean±SD of biological duplicates.

FIG. 16 shows the analytical HPLC traces of purified linear and CHD peptides.

FIG. 17 shows the chemical structures of Sos CHDs.

### DETAILED DESCRIPTION OF THE INVENTION

The following detailed description is presented to enable any person skilled in the art to make and use the subject technology. For purposes of explanation, specific nomenclature is set forth to provide a thorough understanding of the present invention. However, it will be apparent to one skilled in the art that these specific details are not required to practice the technology. Descriptions of specific applications are provided only as representative examples. The present technology is not intended to be limited to the embodiments shown, but is to be accorded the widest possible scope consistent with the principles and features disclosed herein. Preferences and options for a given aspect, feature, or parameter of the invention should, unless the context indicates otherwise, be regarded as having been disclosed in combination with any and all preferences and options for all other aspects, features, and parameters of the invention.

The present invention provides a synthetic Sos protein mimic that engages the wild-type and oncogenic forms of nucleotide-bound Ras and modulates downstream kinase signaling. The Sos mimic was designed to capture the conformation of the Sos helix-loop-helix motif that makes critical contacts with Ras in its switch region. Chemoproteomics studies illustrate that the proteomimetic engages Ras and other cellular GTPases. In some embodiments, the synthetic proteomimetic resists proteolytic degradation and enters cells through macropinocytosis. As such, it is selectively toxic to cancer cells with upregulated macropinocytosis, including those that feature oncogenic Ras mutations. The present invention provides the design of a conformationally-defined

proteomimetic that reproduces a key binding surface of Sos, a well-characterized effector of Ras. The proteomimetic binds wild-type Ras and its various mutant forms and downregulates Ras signaling. Significantly and surprisingly, the compound shows enhanced internalization into cancer cells that upregulate macropinocytosis.

The present invention hypothesizes that the introduction of additional contact

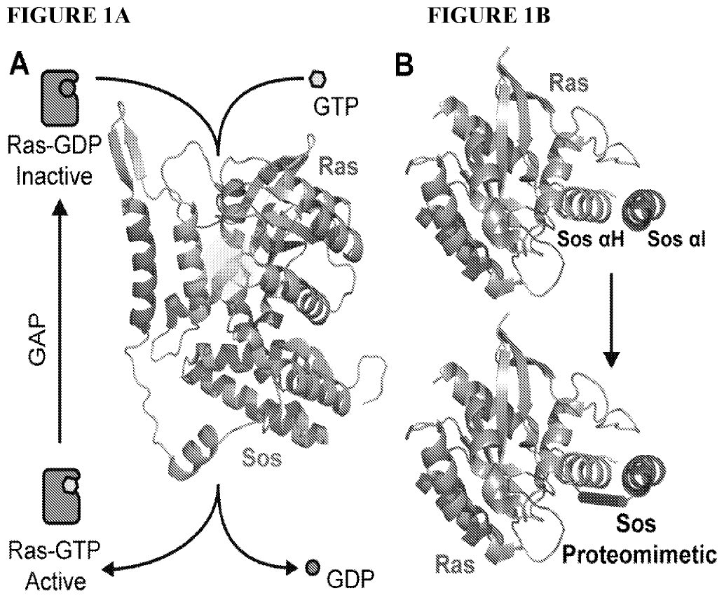

residues from Sos may allow engagement of nucleotide-bound Ras (see FIG. 1B). Sos inserts αH into the switch region of Ras, but analysis of the complex shows that several other residues from Sos also interact with Ras (FIG. 6). The conformation of αH helix, itself, is controlled by the αI domain as part of a hairpin helix organization. The αI helix makes important electrostatic contacts with the Ras effector loop in the switch I region.

The present invention develops a tertiary structure mimic of Sos that encompasses critical binding residues from the helix-loop-helix motif to determine if the additional contacts allow engagement of nucleotide bound Ras (see FIG. 1B). A recently described synthetic approach has been utilized to capture the conformation of the Sos αH and αI helices. In prior efforts, it was observed that helix dimers may be stabilized by judicious substitution of a surface salt bridge with a covalent bond and appropriate sculpting of the dimeric interface to coerce knob-into-hole helix packing (M. G. Wuo, S. H. Hong, A. Singh, P. S. Arora, Synthetic Control of Tertiary Helical Structures in Short Peptides. J. Am. Chem. Soc. 140, 16284-16290 (2018); M. G. Wuo, A. B. Mahon, P. S. Arora, An Effective Strategy for Stabilizing Minimal Coiled Coil Mimetics. J. Am. Chem. Soc. 137, 11618-11621 (2015)). These stabilized proteomimetics are termed crosslinked helix dimers or CHDs. The present invention shows that Sos CHDs are proteolytically stable, selectively cell permeable, and engage the Sos-binding surface of nucleotide-bound Ras with high specificity in biochemical and cellular contexts. The present invention utilizes a combination of rational design principles and computational modeling to exploit previously unexplored and underutilized pockets at the target interface (F. Lauck, C. A. Smith, G. F. Friedland, E. L. Humphris, T. Kortemme, RosettaBackrub—a web server for flexible backbone protein structure modeling and design. Nucleic Acids Res. 38, W569-575 (2010); D. Rooklin et al., Targeting Unoccupied Surfaces on Protein-Protein Interfaces. J. Am. Chem. Soc. 139, 15560-15563 (2017)). The optimized proteomimetic binds wild-type and mutant Ras forms with nanomolar to low micromolar affinities, modulates nucleotide exchange, engages Ras and other Ras subfamily GTPases as demonstrated by chemoproteomics assays, inhibits downstream activation of the Ras-mediated signaling cascade, and is selectively toxic to cancer cells with oncogenic Ras mutations.

Ras remains an intractable target for traditional drug discovery. Multiple strategies to develop lead compounds have been described (R. Spencer-Smith et al., Inhibition of RAS function through targeting an allosteric regulatory site. Nat. Chem. Biol. 13, 62-68 (2017); M. E. Welsch et al., Multivalent Small-Molecule Pan-RAS Inhibitors. Cell 168, 878-889.e₈₂₉ (2017); T. Maurer et al., Small-molecule ligands bind to a distinct pocket in Ras and inhibit SOS-mediated nucleotide exchange activity. Proc. Natl. Acad. Sci. USA 109, 5299-5304 (2012); Q. Sun et al., Discovery of Small Molecules that Bind to K-Ras and Inhibit Sos-Mediated Activation. Angew. Chem. Int. Ed. 51, 6140-6143 (2012); J. M. Ostrem, K. M. Shokat, Direct small-molecule inhibitors of KRAS: from structural insights to mechanism-based design. Nat. Rev. Drug Discov. 15, 771-785 (2016); J. Downward, Targeting RAS signalling pathways in cancer therapy. Nat. Rev. Cancer 3, 11-22 (2003); A. G. Stephen, D. Esposito, R. K. Bagni, F. McCormick, Dragging ras back in the ring. Cancer Cell 25, 272-281 (2014)), but with the exception of covalent G12C inhibitors, these leads have not advanced into clinical trials (B. A. Lanman et al., Discovery of a Covalent Inhibitor of KRAS(G12C) (AMG 510) for the Treatment of Solid Tumors. J. Med. Chem. 63, 52-65 (2020)). Ras-targeting compounds should ideally be able to engage specific mutant isoforms because pan-Ras inhibitors are expected to present unwanted side effects.

The present invention presents a Sos proteomimetic that binds the nucleotide

binding pocket of wild-type and mutant Ras forms but is selectively toxic to oncogenic Ras cells because the cellular internalization of the proteomimetic is governed by macropinocytosis that is upregulated in the mutant Ras cells.

The synthetic derivative mimics a Sos helical hairpin that mediates nucleotide exchange and activation of Ras. The design and synthesis of the proteomimetic is based on a strategy to develop conformationally-defined minimal mimics of helical tertiary structures. The strategy leads to crosslinked helix dimers (CHDs) whose conformational stability requires optimal knob-into-hole helix packing and an appropriately-placed covalent crosslinker. Computational modeling and rational design principles have been utilized to incorporate noncanonical residues to enhance binding interactions of the Sos derivatives with Ras. Several design and sequence iterations were needed in order to develop a lead CHD with the requisite aqueous solubility and low micromolar binding affinity for wild-type and mutant Ras isoforms. Titration ¹H-¹⁵N-HSQC NMR and photoaffinity labeling studies reveal that the lead derivative, CHC^{Sos}-5, engages Ras at its nucleotide binding surface.

CHC^{Sos}-5 proved to be cell-permeable and exhibited similar uptake as the well-characterized cell-penetrating Tat peptide. Significantly, CHC^{Sos}-5 displayed higher cellular uptake in Ras mutant cells relative to those with only the wild-type variant. This superior cellular internalization result in mutant Ras cells is consistent with the recent characterization of the uptake pathways for peptidomimetics—as it has recently been discovered that macropinocytosis is a key mechanism utilized by medium-sized peptidic compounds. The upregulation of macropinocytosis in cell lines carrying mutations in Ras serves as a selectivity filter for the Sos proteomimetic to specifically engage oncogenic Ras even though its binding affinity is slightly better for wild-type Ras in biochemical experiments.

The potential of CHC^{Sos}-5 to modulate phosphorylation of ERK—a well-documented downstream kinase impacted by Ras activation—was assayed. CHC^{Sos}-5 proved to be a potent inhibitor of ERK with IC₅₀<1 μM, in the mutant Ras cell line but not in cell line expressing wild-type Ras. Significantly, the alanine control CHC^{Sos}-3, did not modulate ERK phosphorylation at the concentrations tested. This result suggests that the efficacy of the lead proteomimetic is sequence specific. Lastly, photoaffinity labeling integrated with quantitative MS-based proteomics was employed to map interactors for CHC^{Sos}-5 in an oncogenic Ras cell line and identified K-Ras and other Ras family members. The fact that CHC^{Sos}-5 engages proteins other than Ras may reflect unknown natural partners of Sos in the absence its membrane recruitment.

While there have been many successful proteomics studies utilizing small

molecules and peptides to interrogate protein-protein interactions, (R. E. Kleiner, L. E. Hang, K. R. Molloy, B. T. Chait, T. M. Kapoor, A Chemical Proteomics Approach to Reveal Direct Protein-Protein Interactions in Living Cells. Cell Chemical Biology 25, 110-120.e₁₁₃ (2018); D. P. Murale, S. C. Hong, M. M. Haque, J.-S. Lee, Photo-affinity labeling (PAL) in chemical proteomics: a handy tool to investigate protein-protein interactions (PPIs). Proteome Science 15, 14 (2017)) the use of protein tertiary mimics to map binding partners of therapeutically important protein domains has not been previously described. The biological relevance of targeting previously unknown binding partners of Sos helical hairpin remains to be deciphered. In summary, the present invention provides a pan-Ras ligand that is selectively toxic to cells that express mutant Ras isoforms and upregulate macropinocytosis as a nutrient uptake pathway. The selective uptake of the Sos proteomimetic suggests a therapeutic strategy for targeting oncogenic Ras without the requirement for ligands that specifically engage its various mutations.

One aspect of the present invention relates to a macrostructure. This macrostructure includes an antiparallel coiled-coil, wherein the antiparallel coiled-coil comprises:

a first coil of Formula I and a second coil of Formula II:

T₁-g₀-a₁-b₁-c₁-d₁-e₁-f₁-g₁-a₂-b₂-c₂-d₂-e₂-f₂-g₂-a₃-b₃-c₃-d₃-e₃-f₃-T₂ (I)

T₃-f′₀-g′₀-a′₁-b′₁-c′₁-d′₁-e′₁-f′₁-g′₁-a′₂-b′₂-c′₂-d′₂-e′₂-f′₂-g′₂-a′₃-b′₃-c′₃-d′₃-e′₃-T₄ (II),
wherein:

each a₁₋₃, b₁₋₃, c₁₋₃, d₁₋₃, e₁₋₃, f₁₋₃, g₀₋₂, a′₁₋₃, b′₁₋₃, c′₁₋₃, d′₁₋₃, e′₁₋₃, f′₀₋₂, and g′₀₋₂ is independently absent or a residue selected from the group consisting of modified or unmodified amino acid residues and analogues thereof;

one or more of the following residue pairs are covalently bound by a linker: g₀-g′₂, g₁-g′₁, g₂-g′₀, a₁-d′₃, a₂-d′₂, a₃-d′₁, d₁-a′₃, d₂-a′₂, d₃-a′₁, e₁-e′₃, e₂-e′₂, and e₃-e′₁;

each T₁ and T₃ is independently a point of attachment from a terminal nitrogen to one or more (preferably one or two) moieties, wherein each moiety is independently H, -PG₁, —C(O)R, —C(O)NR₂, —C(O)NH₂, —R, —C(O)OR, an amino acid or analogue thereof, a peptide, a targeting moiety, or a tag, where PG₁ is an amine protecting group and each R is independently hydrogen, an alkyl, an alkenyl, an alkynyl, a cycloalkyl, an aryl, a heteroaryl, a heterocyclyl, an arylalkyl, a peptide, a targeting moiety, or a tag; and

each T₂ and T₄ is independently a point of attachment from a terminal carbonyl to H, —OPG₂, —NPG₂, —OR, —OH, -NR₂, —NH₂, —N(R)C(O)C₁₋₆ alkyl, —N(H)C(O)C₁₋₆ alkyl, an amino acid or analogue thereof, a peptide, a targeting moiety, or a tag, where PG₂ is a carboxylic acid protecting group and each R is independently hydrogen, an alkyl, an alkenyl, an alkynyl, a cycloalkyl, an aryl, a heteroaryl, a heterocyclyl, an arylalkyl, a peptide, a targeting moiety, or a tag;
and wherein:

the first coil comprises at least ten contiguous residues, wherein the at least ten contiguous residues have the formula X₁-X₂-X₃-X₄-X₅-X₆-X₇-X₈-X₉-X₁₀-X₁₁-X₁₂-X₁₃-X₁₄-X₁₅-X_{16;}

the second coil comprises at least ten contiguous residues, wherein the at least ten contiguous residues have the formula X′₁-X′₂-X′₃-X′₄-X′₅-X′₆-X′₇-X′₈-X′₉-X′₁₀-X′₁₁-X′₁₂-X′₁₃-X′₁₄-X′₁₅-X′₁₆; and

wherein each residue is selected from the groups indicated below (superscript letters indicate each residue's location within Formula I and Formula II; residues in the a, a′, d, d′, e, e′, g, and g′ positions can optionally be modified to facilitate attachment of a linker or replaced with a linker; underlined residues are particularly preferred)

| First Coil | | | Second Coil | | |
|---|---|---|---|---|---|
| | | Preferred | | | Preferred |
| Residue | Group | Residue(s) | Residue | Group | Residue(s) |
| ^{g}X₁ | Any residue | Phe, Trp | | | |
| ^{a}X₂ | Any | Cys, HCys, Leu, | ^{d}X′₁ | Any | Cys, HCys, Leu, |
| | hydrophobic | Ile, allylleucine, | | hydrophobic | Ile, allylleucine, |
| | residue | Val, allylglycine, | | residue | Val, allylglycine, |
| | | Thr, | | | Thr, |
| | | selenocysteine, | | | selenocysteine, |
| | | hexafluoroleucine, | | | hexafluoroleucine, |
| | | hexafluorovaline | | | hexafluorovaline |
| | | (or analogue of | | | (or analogue of |
| | | any of the | | | any of the |
| | | preceding | | | preceding |
| | | residues) | | | residues) |
| ^{b}X₃ | Any residue | Gly | ^{e}X′₂ | Any residue | Ala |
| ^{c}X₄ | Any residue | Arg | ^{f}X′₃ | Any residue | Trp |
| ^{d}X₅ | Any | Cys, HCys, Leu, | ^{g}X′₄ | Any residue | Arg |
| | hydrophobic | Ile, allylleucine, | | | |
| | residue | Val, allylglycine, | | | |
| | | Thr, | | | |
| | | selenocysteine, | | | |
| | | hexafluoroleucine, | | | |
| | | hexafluorovaline | | | |
| | | (or analogue of | | | |
| | | any of the | | | |
| | | preceding | | | |
| | | residues) | | | |
| ^{e}X₆ | Any residue | Cys | ^{a}X′₅ | Any | Cys, HCys, Leu, |
| | | | | hydrophobic | Ile, allylleucine, |
| | | | | residue | Val, allylglycine, |
| | | | | | Thr, |
| | | | | | selenocysteine, |
| | | | | | hexafluoroleucine, |
| | | | | | hexafluorovaline |
| | | | | | (or analogue of |
| | | | | | any of the |
| | | | | | preceding |
| | | | | | residues) |
| ^{f}X₇ | Any residue | Thr | ^{b}X′₆ | Any residue | Arg |
| ^{g}X₈ | Any residue | Glu, Carboxylic | ^{c}X′₇ | Any residue | Glu |
| | | Acid Isostere | | | |
| ^{a}X₉ | Any | Cys, HCys, Leu, | ^{d}X′₈ | Any | Cys, HCys, Leu, |
| | hydrophobic | Ile, allylleucine, | | hydrophobic | Ile, allylleucine, |
| | residue | Val, allylglycine, | | residue | Val, allylglycine, |
| | | Thr, | | | Thr, |
| | | selenocysteine, | | | selenocysteine, |
| | | hexafluoroleucine, | | | hexafluoroleucine, |
| | | hexafluorovaline | | | hexafluorovaline |
| | | (or analogue of | | | (or analogue of |
| | | any of the | | | any of the |
| | | preceding | | | preceding |
| | | residues) | | | residues) |
| ^{b}X₁₀ | Any residue | Leu, L- | ^{e}X′₉ | Any residue | Glu |
| | | homoarginine | | | |
| ^{c}X₁₁ | Any residue | Lys, Arg | ^{f}X′₁₀ | Any residue | Arg |
| ^{d}X₁₂ | any | Cys, HCys, Leu, | ^{g}X′₁₁ | Any residue | Glu |
| | hydrophobic | Ile, allylleucine, | | | |
| | residue | Val, allylglycine, | | | |
| | | Thr, | | | |
| | | selenocysteine, | | | |
| | | hexafluoroleucine, | | | |
| | | hexafluorovaline | | | |
| | | (or analogue of | | | |
| | | any of the | | | |
| | | preceding | | | |
| | | residues) | | | |
| ^{e}X₁₃ | Any residue | Arg | ^{a}X′₁₂ | Any | Cys, HCys, Leu, |
| | | | | hydrophobic | Ile, allylleucine, |
| | | | | residue | Val, allylglycine, |
| | | | | | Thr, |
| | | | | | selenocysteine, |
| | | | | | hexafluoroleucine, |
| | | | | | hexafluorovaline |
| | | | | | (or analogue of |
| | | | | | any of the |
| | | | | | preceding |
| | | | | | residues) |
| ^{f}X₁₄ | Any residue | Glu, Asn, Amide | ^{b}X′ ₁₃ | Any residue | Ala |
| | | isostere, | | | |
| | | Carboxylic acid | | | |
| | | isostere | | | |
| ^{g}X₁₅ | Any residue | Gly | ^{c}X′₁₄ | Any residue | Arg |
| ^{a}X₁₆ | Any residue | Asn, Amide | ^{d}X′₁₅ | Any | Cys, HCys, Leu, |
| | | isostere | | hydrophobic | Ile, allylleucine, |
| | | | | residue | Val, allylglycine, |
| | | | | | Thr, |
| | | | | | selenocysteine, |
| | | | | | hexafluoroleucine, |
| | | | | | hexafluorovaline |
| | | | | | (or analogue of |
| | | | | | any of the |
| | | | | | preceding |
| | | | | | residues) |
| | | | ^{e}X′₁₆ | Any residue | Cys |

Antiparallel coiled-coil structures have a first amino acid strand (or first coil) and a second amino acid strand (or second coil). As will be readily apparent to the skilled artisan, the following conventions are commonly used to characterize coiled-coil structures and are used throughout this application. The convention “A/B” or “^{x}A^{y}/*^{x′}B^{y′}” is used to identify the sequence of each strand (either specifically or generically), where A is the sequence (X₁-X₂-X₃ . . . ) of the first strand, B is the sequence (X₁′-X₂′-X₃′ . . . ) of the second strand, x, x′, y, and y′ identify the starting (x, x′) and ending (y, y′) locations of the corresponding sequences relative to heptad(s) in each strand, and “/” separates one sequence from the other. Conventionally, the A and B sequences are both written, left to right, in an N-to-C orientation. However, as will be readily apparent to the skilled artisan, the strands in an antiparallel coiled-coil structure are spatially aligned in opposite directions, e.g., in a top view taken perpendicular to the axis of an antiparallel coiled-coil, the N-terminal of the first strand will be top-most and the C-terminal of the second strand will be top-most. As will be readily apparent to the skilled artisan, in the compounds of the present invention, there is also at least one covalent linker between a residue in the first strand and a residue in the second strand. The location and structure of the linker(s) are sometimes identified using “Z” and “Z”' in place of X and X′, respectively, in the A and B sequences. Alternatively, the location and structure of the linker(s) are identified by additional explanation (e.g., “there is a disulfide linker between residue Xₙ and residue Xₙ”).

In at least one embodiment, the number of residues in the first and second coils is from 10 to 30 (e.g., in a range having a lower value of 10, 11, 12, 13, 14, 15, 16, 17, 18, 19, 20, 21, 22, 23, 24, 25, 26, 27, 28, or 29 and an upper value of 11, 12, 13, 14, 15, 16, 17, 18, 19, 20, 21, 22, 23, 24, 25, 26, 27, 28, 29, or 30, in any combination).

As will be readily apparent to the skilled artisan, the helical wheel views herein show the spatial orientation of each coil in the antiparallel coiled-coil structure, while the two-dimensional views show the connections between residues.

As used above, and throughout the description of the invention, the following

terms, unless otherwise indicated, shall be understood to have the following meanings. If not defined otherwise herein, all technical and scientific terms used herein have the same meaning as is commonly understood by one of ordinary skill in the art to which this invention belongs. In the event that there is a plurality of definitions for a term herein, those in this section prevail unless stated otherwise.

As used herein, the term “alkyl” means an aliphatic hydrocarbon group which may be straight or branched having about 1 to about 8 (e.g., 1-2, 1-3, 1-4, 1-5, 1-6, 1-7, 1-8) carbon atoms in the chain. Branched means that one or more lower alkyl groups such as methyl, ethyl, or propyl are attached to a linear alkyl chain. Exemplary alkyl groups include methyl, ethyl, n-propyl, i-propyl, n-butyl, t-butyl, n-pentyl, and 3-pentyl.

The term “alkenyl” means an aliphatic hydrocarbon group containing a carbon-carbon double bond and which may be straight or branched having about 2 to about 8 (e.g., 2-3, 2-4, 2-5, 2-6, 2-7, 2-8) carbon atoms in the chain. Preferred alkenyl groups have 2 to about 4 carbon atoms in the chain. Exemplary alkenyl groups include ethenyl, propenyl, n-butenyl, and i-butenyl.

The term “alkynyl” means an aliphatic hydrocarbon group containing a carbon-carbon triple bond and which may be straight or branched having about 2 to about 8 (e.g., 2-3, 2-4, 2-5, 2-6, 2-7, 2-8) carbon atoms in the chain. Preferred alkynyl groups have 2 to about 4 carbon atoms in the chain. Exemplary alkynyl groups include ethynyl, propynyl, n-butynyl, 2-butynyl, 3-methylbutynyl, and n-pentynyl.

The term “amide isostere” refers to an analog that preserves the hydrogen bonding properties of amides without their metabolic susceptibility. The side shains of asparagine and glutamine residues may be modified to 1,2,3-triazole, 1,2,4-triazole, oxazole, oxadiazole, imidazole, thiazole, thiadiazole, tetrazole, pyrazole, indole, pyrrole, pyridine, pyrazine, diketopiperazine, urea, thiourea, carbamate, and sulfonamide. Suitable amide isosteres include those described in Kumari et al., “Amide Bond Bioisosteres: Strategies, Synthesis, and Successes,” J. Med. Chem. 63: 12290-358 (2020), which is hereby incorporated by reference in its entirety.

The term “carboxylic acid isostere” refers to phosphonic acid, sulfonic acids, sulfonamides, sulfonyl urea, hydroxamic acid, acylurea, tetrazole, thiazolidinedione, phenols, squaric acid, thiozolidine dione, oxazolidine dione, oxadiazol-5(4H)-one, thiadiazol-5(4H)-one, oxadiazol-5(4H)-thione, oxathiadiazole-2-oxide, isoxazole, tetramic acid, and cyclopentane 1,3-diones. Suitable carboxylic acid derivatives include those described in Lassalas et al., “Structure Property Relationships of Carboxylic Acid Isosteres,” J. Med. Chem. 59: 3183-203 (2016), which is hereby incorporated by reference in its entirety.

As used herein, the term “cycloalkyl” refers to a non-aromatic saturated or unsaturated mono- or polycyclic ring system which may contain 3 to 8 (3, 4, 5, 6, 7, 8, 3-4, 3-5, 3-6, 3-7, 4-5, 4-6, 4-7, 4-8, 5-6, 5-7, 5-8, 6-7, 6-8, 7-8) carbon atoms, and which may include at least one double bond. Exemplary cycloalkyl groups include, without limitation, cyclopropyl, cyclobutyl, cyclopentyl, cyclohexyl, cyclopropenyl, cyclobutenyl, cyclopentenyl, cyclohexenyl, anti-bicyclopropane, or syn-bicyclopropane.

As used herein, the term “alkane” refers to aliphatic hydrocarbons of formula CₙH₂ₙ₊₂, which may be straight or branched having about 1 to about 8 (e.g., 1-2, 1-3, 1-4, 1-5, 1-6, 1-7, 1-8) carbon atoms in the chain. Branched means that one or more lower alkyl groups such as methyl, ethyl, or propyl are attached to a linear alkyl chain. Exemplary alkanes include methane, ethane, n-propane, i-propane, n-butane, t-butane, n-pentane, and 3-pentane. The term “alkylene” refers to a divalent group formed from an alkane by removal of two hydrogen atoms. Exemplary alkylene groups include, but are not limited to, divalent groups derived from the alkanes described above.

As used herein, the term “alkene” refers to aliphatic unsaturated hydrocarbons of formula CₙH₂ₙ, which may be straight or branched having about 2 to about 8 (e.g., 2-3, 2-4, 2-5, 2-6, 2-7, 2-8) carbon atoms in the chain. Exemplary alkenes include ethylene, propylene, n-butylene, and i-butylene. The term “alkenylene” refers to a divalent group formed from an alkene by removal of two hydrogen atoms. Alkenylenes contain a carbon-to-carbon double bond and are represented by the formula —(CₙH₂ₙ₋₂)—. Exemplary alkenylene groups include, but are not limited to, divalent groups derived from the alkenes described above.

As used herein, the term “alkyne” refers to aliphatic unsaturated hydrocarbons of formula CₙH₂ₙ₋₂, which may be straight or branched having about 2 to about 8 (e.g., 2-3, 2-4, 2-5, 2-6, 2-7, 2-8) carbon atoms in the chain. Exemplary alkynes include acetylene, propyne, butyne, and pentyne. The term “alkynylene” refers to a divalent groups formed from alkynes by removal of two hydrogen atoms. Alkynylene contains a carbon-to-carbon triple bond and is represented by the formula —(CₙH₂ₙ₋₄)—. Exemplary alkynylene groups include, but are not limited to, divalent groups derived from the alkynes described above.

Aromatic rings and heteroaromatic rings can be any single, multiple, or fused ring structures. For example, aromatic or heteroaromatic rings include 5- or 6-membered aromatic or heteroaromatic rings containing 0-3 (0, 1, 2, or 3) heteroatoms selected from O, N, and S; a bicyclic 9- or 10-membered aromatic or heteroaromatic ring system containing 0-3 (0, 1, 2, or 3) heteroatoms selected from O, N, and S; or a tricyclic 13- or 14-membered aromatic or heteroaromatic ring system containing 0-3 (0, 1, 2, or 3) heteroatoms selected from O, N, and S. Aromatic 5- to 14-membered (5-, 6-, 7-, 8-, 9-, 10-, 11-, 12-, 13-, or 14-membered) carbocyclic rings include, e.g., cyclopenta-1,3-diene, benzene, naphthalene, indane, tetralin, and anthracene. 5- to 10-Membered (5-, 6-, 7-, 8-, 9-, or 10-membered) aromatic heterocyclic rings include, e.g., imidazole, pyridine, indole, thiophene, benzopyranone, thiazole, furan, benzimidazole, quinoline, isoquinoline, quinoxaline, pyrimidine, pyrazine, tetrazole, pyrazole, benzimidazole, pyridazine, pyrrole, imidazole, oxazole, isooxazole, indazole, isoindole, imidazole, purine, triazine, quinazoline, cinnoline, benzoxazole, acridine, benzisooxazole, and benzothiazole. The term “arylene” refers to a divalent group derived from an aromatic ring by removal of a hydrogen atom from two ring carbon atoms. Exemplary arylene groups include, but are not limited to, divalent groups derived from the aromatic rings described above. The term “heteroarylene” refers to a divalent group derived from a heteroaromatic ring. Exemplary heteroarylene groups include, but are not limited to, divalent groups derived from the heteroaromatic rings described above.

The term “ether” means a group having the formula —R—O—R—. Each R can be independently selected from the group consisting of a bond, C₁₋₈ alkylene, C₂₋₈ alkenylene, arylene, and heteroarylene. Exemplary ethers include, but are not limited to, -C₁₋₈ alkylene-O-C₁₋₈ alkylene- (e.g., —(CH₂)₂—O—(CH₂)₂—), -C₂₋₈ alkenylene-O-C₂₋₈ alkenylene-, -arylene-O-arylene-, -heteroarylene-O-heteroarylene-, and -C₁₋₈ alkylene-O-heteroarylene-.

The term “thioether” means a group having the formula —R—S—R—. Each R can be independently selected from the group consisting of a bond, C₁₋₈ alkylene, C₂₋₈ alkenylene, arylene, and heteroarylene. Exemplary thioethers include, but are not limited to, -C₁₋₈ alkylene-S-C₁₋₈ alkylene- (e.g., —(CH₂)₂—S—(CH₂)₂—), -C₂₋₈ alkenylene-S-C₂₋₈ alkenylene-, -arylene-S-arylene-, -heteroarylene-S-heteroarylene-, and -C₁₋₈ alkylene-S-heteroarylene-.

The term “amide” means a group having the formula —C(O)N(R¹)(R¹) or —C(O)N(R¹)—. Amides include, e.g., —C(O)N(R¹)R—, —R—C(O)N(R¹)R—, —CHR¹—C(O)N(R¹)R—, and —C(R¹)(R¹)—C(O)N(R¹)R—. Each R can be independently selected from the group consisting of a bond, C₁₋₈ alkylene, C₂₋₈ alkenylene, arylene, and heteroarylene, and each R¹ can be independently selected from the group consisting of hydrogen, C₁₋₈ alkyl, C₂₋₈ alkenyl, C₂₋₈ alkynyl, C³⁻⁸ cycloalkyl, aryl, heteroaryl, heterocyclyl, and arylalkyl. Exemplary amides include, but are not limited to, -C₁₋₈ alkylene-C(O)N(aryl)-, -C₂₋₈ alkenylene-C(O)N(aryl)-, and-C₁₋₈ alkylene-C(O)N(C₁₋₈ alkyl)- (e.g., —(CH₂)₂—C(O)N(CH₃)—).

The term “ester” means a group having the formula —C(O)O—. Esters include, e.g., —R—C(O)O—R—, —CHR¹—C(O)O—R—, and —C(R¹)(R¹)—C(O)O—R—. Each R can be independently selected from the group consisting of a bond, C₁₋₈ alkylene, C₂₋₈ alkenylene, arylene, and heteroarylene, and each R¹ can be independently selected from the group consisting of hydrogen, C₁₋₈ alkyl, C₂₋₈ alkenyl, C₂₋₈ alkynyl, C₃₋₈ cycloalkyl, aryl, heteroaryl, heterocyclyl, and arylalkyl. Exemplary esters include, but are not limited to, -C₁₋₈ alkylene-C(O)O-arylene-, -C₂₋₈ alkenylene-C(O)O-arylene-, -C₁₋₈ alkylene-C(O)O-heteroarylene-, -C₁₋₈ alkylene-C(O)O-C₁₋₈ alkylene- (e.g., —(CH₂)₂—C(O)O—(CH₂)₂—), and -C₁₋₈ alkylene-C(O)O—(e.g., —(CH₂)₂—C(O)O—).

As used herein, the term “heterocyclyl” refers to a stable 3- to 18-membered (3-, 4-, 5-, 6-, 7-, 8-, 9-, 10-, 11-, 12-, 13-, 14-, 15-, 16-, 17-, or 18-membered) ring system that consists of carbon atoms and from one to five (1, 2, 3, 4, 5, 1-2, 1-3, 1-4, 2-3, 2-4, 2-5, 3-4, 3-5, 4-5) heteroatoms selected from the group consisting of nitrogen, oxygen, and sulfur. The heterocyclyl may be a monocyclic or a polycyclic ring system, which may include fused, bridged, or spiro ring systems; and the nitrogen, carbon, or sulfur atoms in the heterocyclyl may be optionally oxidized; the nitrogen atom may be optionally quaternized; and the ring may be partially or fully saturated. Representative monocyclic heterocyclyls include piperidine, piperazine, pyrimidine, morpholine, thiomorpholine, pyrrolidine, tetrahydrofuran, pyran, tetrahydropyran, oxetane, and the like. Representative polycyclic heterocyclyls include indole, isoindole, indolizine, quinoline, isoquinoline, purine, carbazole, dibenzofuran, chromene, xanthene, and the like.

As used herein, the term “aryl” refers to an aromatic monocyclic or polycyclic ring system containing from 6 to 19 (6, 7, 8, 9, 10, 11, 12, 13, 14, 15, 16, 17, 18, 19, 6-7, 6-8, 6-9, 6-10, 6-11, 6-12, 6-13, 6-14, 6-15, 6-16, 1-17, 6-18, 7-8, 7-9, 7-10, 7-11, 7-12, 7-13, 7-14, 7-15, 7-16, 7-18, 7-19, 8-9, 8-10, 8-11, 8-12, 8-13, 8-14, 8-15, 8-16, 8-17, 8-18, 8-19, 9-10, 9-11, 9-12, 9-13, 9-14, 9-15, 9-16, 9-17, 9-18, 9-19, 10-11, 10-12, 10-13, 10-14, 10-15, 10-16, 10-17, 10-18, 10-19, 11-12, 11-13, 11-14, 11-15, 11-16, 11-17, 11-18, 11-19, 12-13, 12-14, 12-15, 12-16, 12-17, 12-18, 12-19, 13-14, 13-15, 13-16, 13-17, 13-18, 13-19, 14-15, 14-16, 14-17, 14-18, 14-19, 15-16, 15-17, 15-18, 15-19, 16-17, 16-18, 16-19, 17-18, 17-19, 18-19) carbon atoms, where the ring system may be optionally substituted. Aryl groups of the present invention include, but are not limited to, groups such as phenyl, naphthyl, azulenyl, phenanthrenyl, anthracenyl, fluorenyl, pyrenyl, triphenylenyl, chrysenyl, and naphthacenyl.

As used herein, “heteroaryl” refers to an aromatic ring radical which consists of

carbon atoms and from one to five heteroatoms selected from the group consisting of nitrogen, oxygen, and sulfur. Examples of heteroaryl groups include, without limitation, pyrrolyl, pyrazolyl, imidazolyl, triazolyl, furyl, thiophenyl, oxazolyl, isoxazolyl, thiazolyl, isothiazolyl, oxadiazolyl, thiadiazolyl, pyridyl, pyrazinyl, pyrimidinyl, pyridazinyl, triazinyl, thienopyrrolyl, furopyrrolyl, indolyl, azaindolyl, isoindolyl, indolinyl, indolizinyl, indazolyl, benzimidazolyl, imidazopyridinyl, benzotriazolyl, benzoxazolyl, benzoxadiazolyl, benzothiazolyl, pyrazolopyridinyl, triazolopyridinyl, thienopyridinyl, benzothiadiazolyl, benzofuyl, benzothiophenyl, quinolinyl, isoquinolinyl, tetrahydroquinolyl, tetrahydroisoquinolyl, cinnolinyl, quinazolinyl, quinolizilinyl, phthalazinyl, benzotriazinyl, chromenyl, naphthyridinyl, acrydinyl, phenanzinyl, phenothiazinyl, phenoxazinyl, pteridinyl, and purinyl. Additional heteroaryls are described in COMPREHENSIVE HETEROCYCLIC CHEMISTRY: THE STRUCTURE, REACTIONS, SYNTHESIS AND USE OF HETEROCYCLIC COMPOUNDS (Katritzky et al. eds., 1984), which is hereby incorporated by reference in its entirety.

The term “arylalkyl” refers to a moiety of the formula —R^{a}R^{b} where R^{a} is an alkyl or cycloalkyl as defined above and R^{b} is an aryl or heteroaryl as defined above.

Compounds described herein may contain one or more asymmetric centers and may thus give rise to enantiomers, diastereomers, and other stereoisomeric forms. Each chiral center may be defined, in terms of absolute stereochemistry, as (R)- or (S)-. This technology is meant to include all such possible isomers, as well as mixtures thereof, including racemic and optically pure forms. Optically active (R)- and (S)-, (−)- and (+)-, or (D)- and (L)-isomers may be prepared using chiral synthons or chiral reagents, or resolved using conventional techniques. When the compounds described herein contain olefinic double bonds or other centers of geometric asymmetry, and unless specified otherwise, it is intended that the compounds include both E and Z geometric isomers. Likewise, all tautomeric forms are also intended to be included.

The term “monocyclic” used herein indicates a molecular structure having one ring.

The term “polycyclic” or “multi-cyclic” used herein indicates a molecular structure having two or more rings, including, but not limited to, fused, bridged, or spiro rings.

The term “optionally substituted” is used to indicate that a group may have a substituent at each substitutable atom of the group (including more than one substituent on a single atom), provided that the designated atom's normal valency is not exceeded and the identity of each substituent is independent of the others. Up to three H atoms in each residue are replaced with alkyl, halogen, haloalkyl, hydroxy, loweralkoxy, carboxy, carboalkoxy (also referred to as alkoxycarbonyl), carboxamido (also referred to as alkylaminocarbonyl), cyano, carbonyl, nitro, amino, alkylamino, dialkylamino, mercapto, alkylthio, sulfoxide, sulfone, acylamino, amidino, phenyl, benzyl, heteroaryl, phenoxy, benzyloxy, or heteroaryloxy. “Unsubstituted” atoms bear all of the hydrogen atoms dictated by their valency. When a substituent is keto (i.e., =0), then two hydrogens on the atom are replaced. Combinations of substituents and/or variables are permissible only if such combinations result in stable compounds; by “stable compound” or “stable structure” is meant a compound that is sufficiently robust to survive isolation to a useful degree of purity from a reaction mixture, and formulation into an efficacious therapeutic agent.

The term “halogen” means fluorine, chlorine, bromine, or iodine.

A “peptide” as used herein is any oligomer of two or more natural or non-natural amino acids, including alpha amino acids, beta amino acids, gamma amino acids, L-amino acids, D-amino acids, and combinations thereof. In preferred embodiments, the peptide is ˜2 to ˜30 (e.g., ˜2 to ˜5, ˜2 to ˜10, ˜5 to ˜10, ˜2 to ˜17, ˜5 to ˜17, ˜10 to ˜17, ˜5 to ˜30, ˜10 to ˜30, or ˜18 to ˜30) amino acids in length. Typically, the peptide is 10-17 amino acids in length. In at least one embodiment, the peptide contains a mixture of alpha and beta amino acids, preferably in the pattern α3/β1.

An amino acid as used herein can be any natural or non-natural amino acid, including alpha amino acids, beta amino acids, gamma amino acids, L-amino acids, and D-amino acids. Amino acid side chains can be any amino acid side chain of such an amino acid.

An amino acid according to the present invention also includes an analogue of a natural or non-natural amino acid. An amino acid analogue is an alpha amino acid with a nonnatural side chain consisting of alkyl, cycloalkyl, aryl, cycloaryl, alkenyl, or alkynyl; or a beta₃-amino acid with a side chain consisting of alkyl, cycloalkyl, aryl, cycloaryl, alkenyl, or alkynyl. As used herein, an amino acid analogue also refers to a natural or nonnatural amino acid that may be substituted for an amino acid residue in the coiled-coil without loss of function relative to the native coiled-coil sequence. Suitable amino acid analogues/substitutions include the natural amino acid substitions described in Betts & Russell, “Amino Acid Properties and Consequences of Substitutions,” in Bioinformatics for Geneticists 289-316 (Michael R. Barnes & Ian C. Gray eds. 2003), which is hereby incorporated by reference in its entirety, as well as the nonnatural substitutions set forth below (all available from Sigma Aldrich) and the nonnatural substitutions described in Gfeller et al., “SwissSidechain: A Molecular and Structural Database of Non-Natural Sidechains,” Nucl. Acids Res. 41: D327-D332 (2013), which is hereby incorporated by reference in its entirety. As will be understood by the skilled artisan, analogues in the table below that are listed as having a protecting group at the N- and/or C-terminal would be deprotected during conjugation to an adjacent residue.

| Amino Acid | Exemplary Non-Natural Analogue(s) |
|---|---|
| Alanine | N-Acetyl-3-(3,4-dimethoxyphenyl)-D-alanine, H-β-Ala-β-naphthalene, Albizziin, (R)-(+)-α- |
| | Allylalanine, (S)-(−)-α-Allylalanine, D-2-Aminobutyric acid, L-2-Aminobutyric acid, DL-2- |
| | Aminobutyric acid, DL-2-Aminobutyric acid, 2-Aminoisobutyric acid, α-Aminoisobutyric |
| | acid, (S)-(+)-2-Amino-4-phenylbutyric acid ethyl ester, Benzyl α-aminoisobutyrate, Boc- |
| | Abu-OH, Boc-D-Abu-OH, Boc-Aib-OH, Boc-β-(9-anthryl)-Ala-OH, Boc-β-(3-benzothienyl)- |
| | Ala-OH, Boc-ß-(3-benzothienyl)-D-Ala-OH, Boc-Cha-OH, Boc-Cha-OMe, Boc-B-(2-furyl)- |
| | Ala-OH, Boc-β-(2-furyl)-D-Ala-OH, Boc-β-iodo-Ala-OBzl, Boc-β-iodo-D-Ala-OBzl, Boc-3- |
| | iodo-D-Ala-OMe, Boc-β-iodo-Ala-OMe, Boc-β-iodo-Ala-OMe, Boc-1-Nal-OH, Boc-D-1-Nal- |
| | OH, Boc-D-1-Nal-OH, Boc-2-Nal-OH, Boc-D-2-Nal-OH, (R)-Boc-3-(2-naphthyl)-β-Ala-OH, |
| | (S)-Boc-3-(2-naphthyl)-β-Ala-OH, Boc-β-phenyl-Phe-OH, Boc-3-(2-pyridyl)-Ala-OH, Boc-3- |
| | (3-pyridyl)-Ala-OH, Boc-3-(3-pyridyl)-D-Ala-OH, (S)-Boc-3-(3-pyridyl)-β-Ala-OH, Boc-3-(4- |
| | pyridyl)-Ala-OH, Boc-3-(4-pyridyl)-D-Ala-OH, Boc-β-(2-quinolyl)-Ala-OH, Boc-3-(2- |
| | quinolyl)-DL-Ala-OH, Boc-3-(3-quinolyl)-DL-Ala-OH, Boc-3-(2-quinoxalyl)-DL-Ala-OH, Boc- |
| | β-(4-thiazolyl)-Ala-OH, Boc-β-(2-thienyl)-Ala-OH, Boc-β-(2-thienyl)-D-Ala-OH, Boc-β-(3- |
| | thienyl)-Ala-OH, Boc-β-(3-thienyl)-D-Ala-OH, 3-Chloro-D-alanine methyl ester, N-[(4- |
| | Chlorophenyl)sulfonyl]-β-alanine, 3-Cyclohexyl-D-alanine, 3-Cyclopentyl-DL-alanine, (−)- |
| | 3-(3,4-Dihydroxyphenyl)-2-methyl-L-alanine, 3,3-Diphenyl-D-alanine, 3,3-Diphenyl-L- |
| | alanine, N-[(S)-(+)-1-(Ethoxycarbonyl)-3-phenylpropyl]-L-alanine, N-[1-(S)-(+)- |
| | Ethoxycarbonyl-3-phenylpropyl]-L-alanyl carboxyanhydride, N-(3-fluorobenzyl)alanine, |
| | Fmoc-Abu-OH, Fmoc-3-(9-anthryl)-Ala-OH, Fmoc-β-azido-Ala-OH, Fmoc-(S)-2-(4- |
| | azidobutane)Ala-OH, Fmoc-(S)-2-(2-azidoethane)Ala-OH, Fmoc-(S)-2-(6-azidohexane)Ala- |
| | OH, Fmoc-(S)-2-(5-azidopentane)Ala-OH, Fmoc-Cha-OH, Fmoc-3-cyclopentyl-DL-Ala-OH, |
| | Fmoc-β-(2-furyl)-Ala-OH, Fmoc-β-(2-furyl)-D-Ala-OH, Fmoc-α-Me-Ala-OH, Fmoc-1-Nal- |
| | OH, Fmoc-D-1-Nal-OH, Fmoc-2-Nal-OH, Fmoc-D-2-Nal-OH, Fmoc-(S)-2-(7-octenyl)Ala-OH, |
| | Fmoc-(R)-2-(pentenyl)Ala-OH, Fmoc-(S)-2-(4-pentenyl)Ala-OH, Fmoc-β-phenyl-Phe-OH, |
| | Fmoc-(R)-2-(2-propenyl)Ala-OH, Fmoc-β-(2-pyridyl)-Ala-OH ≥97.0% (HPLC), Fmoc-β-(2- |
| | pyridyl)-D-Ala-OH, Fmoc-β-(3-pyridyl)-Ala-OH, Fmoc-β-(3-pyridyl)-D-Ala-OH, Fmoc-β-(4- |
| | pyridyl)-Ala-OH, Fmoc-β-(4-pyridyl)-D-Ala-OH, Fmoc-3-(2-quinolyl)-DL-Ala-OH, Fmoc-β-(4- |
| | thiazolyl)-Ala-OH, Fmoc-β-(2-thienyl)-Ala-OH, Fmoc-β-(3-thienyl)-Ala-OH, Fmoc-β-(3- |
| | thienyl)-D-Ala-OH, N-(3-Indolylacetyl)-L-alanine, Methyl (RS)-2-(aminomethyl)-3- |
| | phenylpropionate, 3-(2-Oxo-1,2-dihydro-4-quinolinyl)alanine, 3-(1-Pyrazolyl)-L-alanine, 3- |
| | (2-Pyridyl)-D-alanine, 3-(2-Pyridyl)-L-alanine, 3-(3-Pyridyl)-L-alanine, 3-(4-Pyridyl)-D- |
| | alanine, 3-(4-Pyridyl)-L-alanine, 3-(2-Quinolyl)-DL-alanine, 3-(4-Quinolyl)-DL-alanine, D- |
| | styrylalanine, L-styrylalanine, 3-(2-Thienyl)-L-alanine, 3-(2-Thienyl)-DL-alanine, 3-(2- |
| | Thienyl)-DL-alanine, 3,3,3-Trifluoro-DL-alanine, 3-Ureidopropionic acid, Z-Aib-OH, Z-Cha- |
| | OH, Z-Dehydro-Ala-OMe, Z-dehydro-Ala-OH, Z-D-2-Nal-OH. |
| Isoleucine | Boc-allo-Ile-OH, D-allo-Isoleucine, D-allo-Isoleucine, DL-allo-Isoleucine. |
| Leucine | Homoleucine, N-[(2S,3R)-3-Amino-2-hydroxy-4-phenylbutyryl]-L-leucine, Boc-4,5- |
| | dehydro-Leu-OH, Boc-Ile-Osu, Cycloleucine, N-(3,5-Dinitrobenzoyl)-DL-leucine, Fmoc-tBu- |
| | Gly-OH, N-Formyl-Leu-OH, N-(3-Indolylacetyl)-L-isoleucine, D-tert-Leucine, D-tert- |
| | Leucine, L-tert-Leucine, L-tert-Leucine, DL-tert-Leucine, DL-tert-Leucine, L-tert-Leucine |
| | methyl ester, 5,5,5-Trifluoro-DL-leucine. |
| Valine | 3-Fluoro-DL-valine, 4,4,4,4′,4′,4′-Hexafluoro-DL-valine, (R)-(+)-α-Methylvaline, (S)-(−)-α- |
| | Methylvaline. |
| Phenylalanine | Boc-Homophenylalanine-OH, Boc-D-Homophenylalanine-OH, Fmoc-Homophenylalanine- |
| | OH, Fmoc-D-Homophenylalanine-OH, Z-Homophenylalanine-OH, Boc-(R)-β2- |
| | homophenylalanine, DL-homophenylalanine methyl ester, D-Homophenylalanine, L- |
| | Homophenylalanine, DL-Homophenylalanine, D-Homophenylalanine ethyl ester, Ac-p- |
| | bromo-DL-Phe-OH, (S)-N-acetyl-4-bromophenylalanine, N-Acetyl-2-fluoro-DL- |
| | phenylalanine, N-Acetyl-4-fluoro-DL-phenylalanine, 4-Amino-L-phenylalanine, Boc-4- |
| | azido-Phe-OH, Boc-Bpa-OH, Boc-D-Bpa-OH, Boc-4-tert-butyl-Phe-OH, Boc-4-tert-butyl-D- |
| | Phe-OH, Boc-4-(Fmoc-amino)-L-phenylalanine, rac-Boc-β2-homophenylalanine, (S)-Boc- |
| | 4-methoxy-β-Phe-OH, Boc-2-nitro-L-phenylalanine, Boc-pentafluoro-D-phenylalanine, |
| | Boc-pentafluoro-L-phenylalanine, Boc-Phe(4-Br)-OH, Boc-D-Phe(4-Br)-OH, Boc-Phe(2- |
| | CF3)-OH, Boc-D-Phe(2-CF3)-OH, Boc-Phe(3-CF3)-OH, Boc-D-Phe(3-CF3)-OH, Boc-Phe(4- |
| | CF3)-OH, Boc-D-Phe(4-CF3)-OH, Boc-Phe(2-Cl)-OH, Boc-D-Phe(2-Cl)-OH, Boc-Phe(2,4-Cl2)- |
| | OH, Boc-D-Phe(2,4-Cl2)-OH, Boc-D-Phe(3-Cl)-OH, Boc-Phe(3,4-Cl2)-OH, Boc-D-Phe(3,4- |
| | Cl2)-OH, Boc-Phe(4-Cl)-OH, Boc-D-Phe(4-Cl)-OH, Boc-Phe(2-CN)-OH, Boc-D-Phe(2-CN)- |
| | OH, Boc-Phe(3-CN)-OH, Boc-D-Phe(3-CN)-OH, Boc-Phe(4-CN)-OH, Boc-D-Phe(4-CN)-OH, |
| | Boc-Phe(2-Me)-OH, Boc-D-Phe(2-Me)-OH, Boc-Phe(3-Me)-OH, Boc-D-Phe(3-Me)-OH, Boc- |
| | Phe(4-Me)-OH, Boc-Phe(4-NH2)-OH, Boc-Phe(4-NO2)-OH, Boc-D-Phe(4-NO2)-OH, Boc- |
| | Phe(2-F)-OH, Boc-D-Phe(2-F)-OH, Boc-Phe(3-F)-OH, Boc-D-Phe(3-F)-OH, Boc-Phe(3,4-F2)- |
| | OH, Boc-D-Phe(3,4-F2)-OH, Boc-Phe(3,5-F2)-OH, Boc-Phe(4-F)-OH, Boc-D-Phe(4-F)-OH, |
| | Boc-Phe(4-I)-OH, Boc-D-Phe(4-I)-OH, Boc-D-3,4,5-trifluorophenylalanine, 4-Borono-D- |
| | phenylalanine, 4-Borono-L-phenylalanine, 4-Borono-DL-phenylalanine, p-Bromo-DL- |
| | phenylalanine, 4-Bromo-L-phenylalanine, N-(tert-Butoxycarbonyl)-β-phenyl-D- |
| | phenylalanine, 4-Chloro-L-phenylalanine, DL-2,3-Difluorophenylalanine, DL-3,5- |
| | Difluorophenylalanine, 3,4-Dihydroxy-L-phenylalanine, 3-(3,4-Dimethoxyphenyl)-L- |
| | alanine, N-[(9H-Fluoren-9-ylmethoxy)carbonyl]-2-methoxy-L-phenylalanine, o-Fluoro-DL- |
| | phenylalanine, m-Fluoro-L-phenylalanine, m-Fluoro-DL-phenylalanine, p-Fluoro-D- |
| | phenylalanine, p-Fluoro-D-phenylalanine, p-Fluoro-L-phenylalanine, p-Fluoro-DL- |
| | phenylalanine, 4-Fluoro-D-phenylalanine, 2-fluoro-L-phenylalanine methyl ester, H-p- |
| | fluoro-DL-Phe-OMe, Fmoc-Bpa-OH, Fmoc-D-Bpa-OH, Fmoc-D-3-bromophenylalanine, |
| | Fmoc-D-4-bromophenylalanine, L-Fmoc-β-(6-chloro-4-pyridinyl)alanine, Fmoc-D-3,5- |
| | difluorophenylalanine, L-Fmoc-3-fluorophenylalanine, L-Fmoc-4-fluorophenylalanine, L- |
| | Fmoc-β-(1H-5-indolyl)alanine, Fmoc-2-nitro-L-phenylalanine, Fmoc-pentafluoro-L- |
| | phenylalanine, Fmoc-Phe(4-Boc2-guanidino)-OH, Fmoc-Phe(3-Br)-OH, Fmoc-Phe(4-Br)- |
| | OH, Fmoc-Phe(2-CF3)-OH, Fmoc-D-Phe(2-CF3)-OH, Fmoc-Phe(3-CF3)-OH, Fmoc-D-Phe(3- |
| | CF3)-OH, Fmoc-Phe(4-CF3)-OH, Fmoc-D-Phe(4-CF3)-OH, Fmoc-Phe(2-Cl)-OH, Fmoc-D- |
| | Phe(2-Cl)-OH, Fmoc-Phe(2,4-Cl2)-OH, Fmoc-D-Phe(2,4-Cl2)-OH, Fmoc-Phe(3,4-Cl2)-OH, |
| | Fmoc-D-Phe(3,4-Cl2)-OH, Fmoc-Phe(4-Cl)-OH, Fmoc-D-Phe(4-Cl)-OH, Fmoc-Phe(2-CN)- |
| | OH, Fmoc-D-Phe(2-CN)-OH, Fmoc-Phe(3-CN)-OH, Fmoc-D-Phe(3-CN)-OH, Fmoc-Phe(4- |
| | CN)-OH, Fmoc-Phe(2-Me)-OH, Fmoc-Phe(3-Me)-OH, Fmoc-D-Phe(3-Me)-OH, Fmoc-Phe(4- |
| | NO2)-OH, Fmoc-D-Phe(4-NO2)-OH, Fmoc-D-Phe(2-F)-OH, Fmoc-Phe(3-F)-OH, Fmoc-D- |
| | Phe(3-F)-OH, Fmoc-Phe(3,4-F2)-OH, Fmoc-Phe(3,5-F2)-OH, Fmoc-Phe(4-F)-OH, Fmoc-D- |
| | Phe(4-F)-OH, Fmoc-Phe(4-I)-OH, Fmoc-D-Phe(4-I)-OH, Fmoc-4-(phosphonomethyl)-Phe- |
| | OH, L-Fmoc-4-trifluoromethylphenylalanine, Fmoc-3,4,5-trifluoro-D-phenylalanine, |
| | Fmoc-L-3,4,5-trifluorophenylalanine, 6-Hydroxy-DL-DOPA, 4-(Hydroxymethyl)-D- |
| | phenylalanine, N-(3-Indolylacetyl)-L-phenylalanine, p-Iodo-D-phenylalanine, 4-Iodo-L- |
| | phenylalanine, α-Methyl-D-phenylalanine, α-Methyl-L-phenylalanine, α-Methyl-DL- |
| | phenylalanine, α-Methyl-DL-phenylalanine methyl ester, 4-Nitro-D-phenylalanine, 4- |
| | Nitro-L-phenylalanine, 4-Nitro-DL-phenylalanine, (S)-(+)-4-Nitrophenylalanine methyl |
| | ester, 2-(Trifluoromethyl)-D-phenylalanine, 2-(Trifluoromethyl)-L-phenylalanine, 3- |
| | (Trifluoromethyl)-D-phenylalanine, 3-(Trifluoromethyl)-L-phenylalanine, 4- |
| | (Trifluoromethyl)-D-phenylalanine, 3,3′,5-Triiodo-L-thyronine, Z-L-Phe chloromethyl |
| | ketone. |
| Tryptophan | 5-Fluoro-L-tryptophan, 5-Fluoro-DL-tryptophan, 5-Hydroxy-L-tryptophan, 5-Methoxy-DL- |
| | tryptophan, 5-Methyl-DL-tryptophan tryptophan analog, H-Tpi-Ome. |
| Tyrosine | 3-Amino-L-tyrosine, Boc-3-chloro-D-Tyr-OH, Boc-Tyr(3,5-I2)-Osu, 3-Chloro-L-tyrosine, |
| | Fmoc-Tyr(3-NO2)-OH, Fmoc-Tyr(3,5-I2)-OH, α-Methyl-DL-tyrosine, 3-Nitro-L-tyrosine, 3- |
| | Nitro-L-tyrosine ethyl ester, 3-Nitro-L-tyrosine ethyl ester, DL-o-Tyrosine. |
| Asparagine | Boc-Asn(Xan)-OH, Nα-Boc-Nβ-xanthenyl-L-asparagine. |
| Cysteine | Homocysteine, DL-Homocysteine, L-Homocysteine thiolactone, L-Homocysteine |
| | thiolactone, L-Homocystine, BOC-CYS(ME)-OH, L-Cysteic acid, L-Cysteinesulfinic acid, D- |
| | Ethionine, Fmoc-Cys(Boc-methyl)-OH, Seleno-L-cystine, S-(2-Thiazolyl)-L-cysteine, S-(4- |
| | Tolyl)-L-cysteine. |
| Glutamine | Boc-Cit-OH, D-Citrulline, Fmoc-Cit-OH, Thio-L-citrulline. |
| Serine | Fmoc-Homoser(Trt)-OH, Fmoc-D-Homoser(Trt)-OH, D-Homoserine, L-3-Homoserine, N- |
| | Trityl-L-homoserine, N-Benzoyl-(2R,3S)-3-phenylisoserine, D-Cycloserine, Fmoc-Gly-Val- |
| | OH, Fmoc-Ser[GalNAc(Ac)3-α-D]-OH, L-Isoserine, DL-Isoserine, DL-3-Phenylserine, N-Z-L- |
| | Homoserine lactone. |
| Threonine | Fmoc-Thr[GalNAc(Ac)3-α-D]-OH, L-allo-Threonine, D-Thyroxine. |
| Aspartic acid | (S)-(−)-4-tert-Butyl hydrogen 2-azidosuccinate, N-Z-L-aspartic anhydride. |
| Glutamic acid | (S)-5-tert-Butyl hydrogen 2-azidoglutarate, γ-Carboxy-DL-glutamic acid, 4-Fluoro-DL- |
| | glutamic acid, (4S)-4-(4-Trifluoromethyl-benzyl)-L-glutamic acid. |
| Arginine | L-Homoarginine, L-2-Amino-3-guanidinopropionic acid, L-2-Amino-3-guanidinopropionic |
| | acid hydrochloride, 4-Guanidinobutyric acid, 3-Guanidinopropionic acid. |
| Histidine | N-Boc-3-(3-methyl-4-nitrobenzyl)-L-histidine methyl ester. |
| Lysine | (S)-(−)-1-[N-(1-Ethoxycarbonyl-3-phenylpropyl)-N-trifluoroacetyl]-L-lysine, Fmoc-β- |
| | Lys(Boc)-OH, Fmoc-Lys(palmitoyl)-OH, DL-5-Hydroxylysine, (5R)-5-Hydroxy-L-lysine. |
| Glycine | Fmoc-allyl-Gly-OH, Fmoc-propargyl-Gly-OH, (±)-Boc-α-phosphonoglycine trimethyl ester, |
| | Fmoc-D-propargyl-Gly-OH, Fmoc-D-allyl-Gly-OH, Boc-D-allyl-Gly-OH, Boc-allyl-Gly-OH, |
| | Boc-D-Chg-OH, Boc-Chg-OH, N-Fmoc-iminodiacetic acid, Di-tert-butyl- |
| | iminodicarboxylate, N-Boc-iminodiacetic acid, N-(2-Hydroxyethyl)iminodiacetic acid, |
| | Iminodiacetic acid, Fmoc-N-(1-Boc-4-piperidyl)glycine, N-Lauroylsarcosine, D-α- |
| | Cyclohexylglycine, L-α-Neopentylglycine , L-C-Propargylglycine, Sarcosine, Z-D-Chg-OH, |
| | (±)-Z-α-Phosphonoglycine trimethyl ester, Sarcosine, N-(Phosphonomethyl)glycine, Z-α- |
| | Phosphonoglycine trimethyl ester, N-[Bis(methylthio)methylene]glycine methyl ester, N- |
| | (2-Furoyl)glycine, N-(2-Furfurylideneacetyl)glycine methyl ester, N-(Chloroacetyl)glycine |
| | ethyl ester, Boc-(2-indanyl)-Gly-OH, Fmoc-(2-indanyl)-Gly-OH, Fmoc-N-(2-Boc- |
| | aminoethyl)-Gly-OH, Fmoc-N-(4-Boc-aminobutyl)-Gly-OH, Fmoc-N-(2,4- |
| | dimethoxybenzyl)-Gly-OH, Boc-D-cyclopropylglycine, Boc-(S)-2-thienylglycine, Boc-(R)-2- |
| | thienylglycine, Boc-(S)-3-thienylglycine, Boc-(R)-3-thienylglycine, Boc-L- |
| | cyclopropylglycine, L-α-Cyclopropylglycine, Boc-propargyl-Gly-OH, D-Allylglycine, |
| | (2S,3R,4S)-α-(Carboxycyclopropyl)glycine, D-Propargylglycine, N-Boc-2-(4- |
| | trifluoromethyl-phenyl)-DL-glycine, Boc-D-propargylglycine, (S)-(+)-2-chlorophenylglycine |
| | methyl ester, (R)-N-Boc-4-fluorophenylglycine, (S)-N-Boc-4-fluorophenylglycine, N-(2- |
| | fluorophenyl)-N-(methylsulfonyl) glycine, N-(4-fluorophenyl)-N-(methylsulfonyl)glycine, |
| | N-(2-chlorophenyl)-N-(methylsulfonyl)glycine, Ethyl acetamidocyanoacetate, N-(4- |
| | Hydroxyphenyl)glycine. |
| Proline | trans-1-Acetyl-4-hydroxy-L-proline, N-[3-(Acetylthio)-(2S)-methylpropionyl]-L-proline, (S)- |
| | α-Allyl-proline, Boc-(S)-α-allyl-Pro-OH, Boc-α-allyl-DL-Pro-OH, N-Boc-cis-4-azido-L-proline, |
| | Boc-(S)-α-benzyl-Pro-OH, Boc-α-(2-bromobenzyl)-DL-Pro-OH, Boc-α-(4-bromobenzyl)-DL- |
| | Pro-OH, Boc-α-(2-chlorobenzyl)-DL-Pro-OH, Boc-α-(3-chlorobenzyl)-DL-Pro-OH, N-Boc-4- |
| | (2,2-difluorocyclopropyl)-L-proline, Boc-α-(diphenylmethyl)-DL-Pro-OH, Boc-(R)-α-(4- |
| | fluorobenzyl)-Pro-OH, Boc-(S)-α-(4-fluorobenzyl)-Pro-OH, Boc-α-(4-fluorobenzyl)-DL-Pro- |
| | OH, N-Boc-cis-4-N-Fmoc-amino-L-proline, N-Boc-trans-4-N-Fmoc-amino-L-proline, N-Boc- |
| | cis-4-hydroxy-D-proline, N-Boc-cis-4-hydroxy-L-proline, N-Boc-trans-4-hydroxy-D-proline, |
| | N-Boc-cis-4-hydroxy-L-proline methyl ester, N-Boc-trans-4-hydroxy-L-proline methyl |
| | ester, N-Boc-4-hydroxy-D-pyrrolidine lactone, N-Boc-4-hydroxy-L-pyrrolidine lactone, |
| | Boc-Hyp(Bzl)-OH, Boc-Hyp-OH, Boc-α-Me-DL-Pro-OH, Boc-α-(4-methylbenzyl)-DL-Pro-OH, |
| | Boc-α-(1-naphthylmethyl)-DL-Pro-OH, N-Boc-2-piperidinecarboxylic acid, (R)-(+)-N-Boc-2- |
| | piperidinecarboxylic acid, Boc-Pip-OH, Boc-α-propyl-DL-Pro-OH, Boc-α-(2-propynyl)-L- |
| | proline, Boc-(R)-4-(2-propynyl)-L-proline, N-Boc-trans-4-(p-tosyloxy)-L-proline methyl |
| | ester, Boc-(R)-4-[2-(trifluoromethyl)benzyl]-L-proline, Boc-(R)-4-[4- |
| | (trifluoromethyl)benzyl]-L-proline, Boc-(R)-α-(4-trifluoromethylbenzyl)-Pro-OH, Boc-(S)-α- |
| | (4-trifluoromethylbenzyl)-Pro-OH, 3,4-Dehydro-L-proline, 3,4-Dehydro-DL-proline, Fmoc- |
| | Hyp-OH, Fmoc-Hyp(tBu)-OH, Fmoc-Pip-OH, Fmoc-D-Pip-OH, cis-3-Hydroxy-DL-proline, |
| | cis-4-Hydroxy-D-proline, cis-4-Hydroxy-L-proline collagen synthesis inhibitor, trans-4- |
| | Hydroxy-D-proline, trans-4-Hydroxy-L-proline, trans-4-Hydroxy-L-proline, L-4-Hydroxy- |
| | proline benzyl ester hydrochloride, L-4-Hydroxyproline methyl ester, (S)-(+)-Methyl |
| | indoline-2-carboxylate, α-Methyl-L-proline, (S)-1-Z-4-oxopyrrolidine-2-carboxylic acid, L- |
| | Pipecolic acid, L-Pipecolic acid Proline homolog, Pipecolinic acid, D-Pipecolinic acid, Z- |
| | Hyp-OH. |

Non-limiting examples of substitutions for certain amino acid residues include, without limitation, those shown below.

| Amino Acid | Examplary Substition |
|---|---|
| Serine | Threonine |
| Tyrosine | Phenylalanine |
| Aspartic acid | Phosphoserine |
| Glutamic acid | Phosphoserine |
| Lysine | arginine, ornithine, diaminoproprionic |
| | acid, diaminobutyric acid |
| Arginine | Lysine |

The amino acids according to the present invention may also be optionally modified. Modifications include, for example, phosphorylation (e.g., phosphoserine, phosphotyrosine, phosphothreonine), halogenation (esp. with 3-9 halogens) (preferably with fluorine, e.g., hexafluoroleucine, hexafluorovaline), methylation (e.g., aspartic acid methyl ester, glutamic acid methyl ester, methyllysine, dimethyllysine, trimethyllysine, dimethylarginine, methylarginine, methyltryptophan), and acetylation (e.g., acetyllysine).

In at least one embodiment of the present invention, at least g₀, a₁, b₁, c₁, d₁, e₁, f₁, g₁, a₂, b₂, c₂, d₂, e₂, f₂, g₂, and a₃, are present in the first coil and at least d′₁, e′₁, f′₁, g′₁, a′₂, b′₂, c′₂, d′₂, e′₂, f′₂, g′₂, a′₃, b′₃, c′₃, d′₃, and e′₃ are present in the second coil.

In at least one embodiment of the present invention, each residue independently has the formula

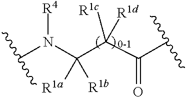

wherein:
R^{1a}, R^{1b}, R^{1c}, and R^{1d} are each independently hydrogen, an amino acid side chain, an alkyl, an alkenyl, an alkynyl, a cycloalkyl, a heterocyclyl, an aryl, a heteroaryl, or an arylalkyl, wherein each amino acid side chain, alkyl, alkenyl, alkynyl, cycloalkyl, heterocyclyl, aryl, heteroaryl, and arylalkyl can be optionally substituted with H, an alkyl, an alkenyl, an alkynyl, an azide, —OR⁵, or —SR⁵; and wherein when a linker covalently binds to a residue, the linker is attached to or replaces one of R^{1a}, R^{1b}, R^{1c}, and R^{1d},
each R⁴ is independently hydrogen, an alkyl, an alkenyl, an alkynyl, a cycloalkyl, a heterocyclyl, an aryl, a heteroaryl, or an arylalkyl; and
each R⁵ is independently selected from the group consisting of H, -PG (where PG is a protecting group), an alkyl, an alkenyl, an alkynyl, a cycloalkyl, an aryl, a heteroaryl, a heterocyclyl, and an arylalkyl.

As will be apparent to the skilled artisan, the linkers in accordance with the present invention create a covalent bridge between an amino acid residue/analogue on one coil of the coiled-coil structure and an amino acid residue/analogue on the other coil in the coiled-coil structure. As will be apparent to the skilled artisan, virtually any covalent linker can be used, provided the appropriate spatial distance between the two linked residues is maintained. The spatial distance as used herein refers to the distance of atoms in the coiled-coil structure when in its solid state, as determined using a static molecular modeling program (e.g., UCSF Chimera) and/or by evaluating the crystal structure of the macrocycle. For linkers between residue pairs g₀-g′₂, g₁-g′₁, g₂-g′₀, e₁-e′₃, e₂-e′₂, and e₃-e′₁ the appropriate spatial distance is 10-25 Å (10-11, 10-12, 10-13, 10-14, 10-15, 10-16, 10-17, 10-18, 10-19, 10-20, 10-21, 10-22, 10-23, 10-24, 11-12, 11-13, 11-14, 11-15, 11-16, 11-17, 11-18, 11-19, 11-20, 11-21, 11-22, 11-23, 11-24, 11-25, 12-13, 12-14, 12-15, 12-16, 12-17, 12-18, 12-19, 12-20, 12-21, 12-22, 12-23, 12-24, 12-25, 13-14, 13-15, 13-16, 13-17, 13-18, 13-19, 13-20, 13-21, 13-22, 13-23, 13-24, 13-25, 14-15, 14-16, 14-17, 14-18, 14-19, 14-20, 14-21, 14-22, 14-23, 14-24, 14-25, 15-16, 15-17, 15-18, 15-19, 15-20, 15-21, 15-22, 15-23, 15-24, 15-25, 16-17, 16-18, 16-19, 16-20, 16-21, 16-22, 16-23, 16-24, 16-25, 17-18, 17-19, 17-20, 17-21, 17-22, 17-23, 17-24, 17-25, 18-19, 18-20, 18-21, 18-22, 18-23, 18-24, 18-25, 19-20, 19-21, 19-22, 19-23, 19-24, 19-25, 20-21, 20-22, 20-23, 20-24, 20-25, 21-22, 21-23, 21-24, 21-25, 22-23, 22-24, 22-25, 23-24, 23-25, or 24-25 Å). In at least one embodiment, the spatial distance is 11-17 Å. In at least one embodiment, the spatial distance is 15-20 Å. For linkers between residue pairs a₁-d′₃, a₂-d′₂, a₃-d′₁, d₁-a′₃, d₂-a′₂, and d₃-a′₁ the appropriate spatial distance is 5-15 Å (5-6, 5-7, 5-8, 5-9, 5-10, 5-11, 5-12, 5-13, 5-14, 6-7, 6-8, 6-9, 6-10, 6-11, 6-12, 6-13, 6-14, 6-15, 7-8, 7-9, 7-10, 7-11, 7-12, 7-13, 7-14, 7-15, 8-9, 8-10, 8-11, 8-12, 8-13, 8-14, 8-15, 9-10, 9-11, 9-12, 9-13, 9-14, 9-15, 10-11, 10-12, 10-13, 10-14, 10-15, 11-12, 11-13, 11-14, 11-15, 12-13, 12-14, 12-15, 13-14, 13-15, or 14-15 Å). In at least one embodiment, the spatial distance is 6-8 Å. In at least one embodiment, the spatial distance is 5-10 Å. Methods of modifying amino acid residues to facilitate attachment of a suitable linker (including replacement of an amino acid side chain with the linker) will also be apparent to the skilled artisan.

In at least one embodiment of the present invention, the length of any linker between residue pairs g₀-g′₂, g₁-g′₁, g₂-g′₀, e₁-e′₃, e₂-e′₂, and e₃-e′₁ is such that the spatial distance between the Cα positions of each residue in the pair is 10-25 Å; and the length of any linker between residue pairs a₁-d′₃, a₁-d′₂, a₃-d′₁, d₁-a′₃, d₂-a′₂, and d₃-a′₁ is such that the spatial distance between the Cα positions of each residue in the pair is 5-15 Å.

In a preferred embodiment, the two amino acids/analogues may be covalently connected to each other using alkylene, alkenylene, arylene, heteroarylene, ethers, thioethers, amides, maleimides, esters, disulfides, diselenides, —O—, —S—, —Se—, and any combination thereof. As will be apparent to the skilled artisan, the linkers may be symmetrical or asymmetrical.

Suitable examples of linkers between residue pairs g₀-g′₂, g₁-g′₁, g₂-g′₀, e₁-e′₃, e₂-e′₂, and e₃-e′₁ include, without limitation, those having the formula —Zn—, wherein n is a number from 1 to 25 (1, 2, 3, 4, 5, 6, 7, 8, 9, 10, 11, 12, 13, 14, 15, 16, 17, 18, 19, 20, 21, 22, 23, 24, 25, or any range within 1 and 25, including, e.g., 1-2, 1-3, 1-4, 1-5, 1-6, 1-7, 1-8, 1-9, 1-10, 1-11, 1-12, 1-13, 1-14, 1-15, 1-16, 1-17, 1-18, 1-19, 1-20, 1-21, 1-22, 1-23, 1-24, 1-25, 2-3, 2-4, 2-5, 2-6, 2-7, 2-8, 2-9, 2-10, 2-11, 2-12, 2-13, 2-14, 2-15, 2-16, 2-17, 2-18, 2-19, 2-20, 2-21, 2-22, 2-23, 2-24, 2-25, 3-4, 3-5, 3-6, 3-7, 3-8, 3-9, 3-10, 3-11, 3-12, 3-13, 3-14, 3-15, 3-16, 3-17, 3-18, 3-19, 3-20, 3-21, 3-22, 3-23, 3-24, 3-25, 4-5, 4-6, 4-7, 4-8, 4-9, 4-10, 4-11, 4-12, 4-13, 4-14, 4-15, 4-16, 4-17, 4-18, 4-19, 4-20, 4-21, 4-22, 4-23, 4-24, 4- 25, 5-6, 5-7, 5-8, 5-9, 5-10, 5-11, 5-12, 5-13, 5-14, 5-15, 5-16, 5-17, 5-18, 5-19, 5-20, 5-21, 5-22, 5-23, 5-24, 5-25, 6-10, 6-15, 6-20, 6-25, 7-10, 7-15, 7-20, 7-25, 8-10, 8-15, 8-20, 8-25, 9-10, 9-15, 9-20, 9-25, 10-15, 10-20, 10-25, 11-15, 11-20, 11-25, 12-15, 12-20, 12- 25, 13-15, 13-20, 13-25, 14-15, 14-20, 14-25, 15-20, 15-25, 16-20, 16-25, 17-20, 17-25, 18-20, 18-25, 19-20, 19-25, 20-25, 21-25, 22-25, 23-25, 24-25; in at least one embodiment, n is 5-25) and each Z is independently selected at each occurrence thereof from the group consisting of alkylene, alkenylene, arylene, heteroarylene (esp. triazole-diyl, thiazole-diyl, oxazole-diyl), ethers, amides, esters, maleimides, thioethers, O, S, and Se. Suitable examples of symmetrical linkers include, without limitation,

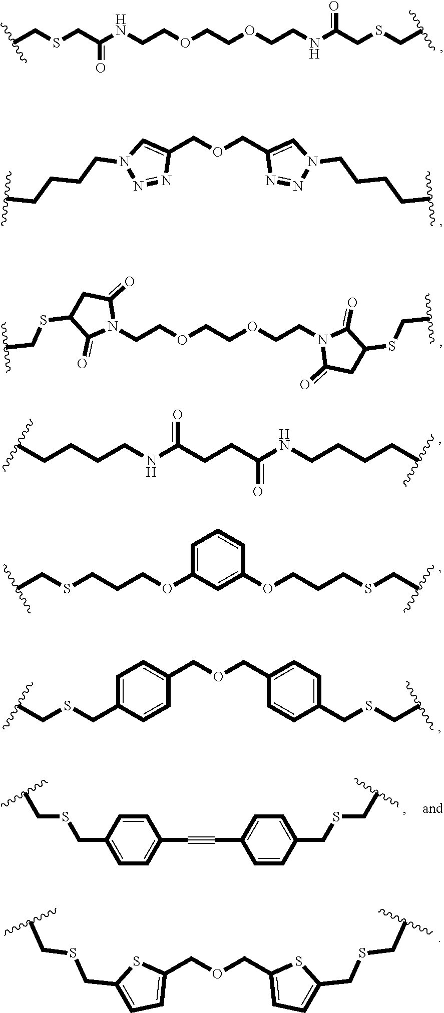

wherein each

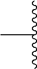

marks a connection point to the Cα carbon in a linked residue/analogue.

In at least one embodiment of the present invention, a linker between at least one of residue pairs g₀-g′₂, g₁-g′₁, g₂-g′₀, e₁-e′₃, e₂-e′₂, and e₃-e′₁ has a formula selected from (a) the group consisting of:

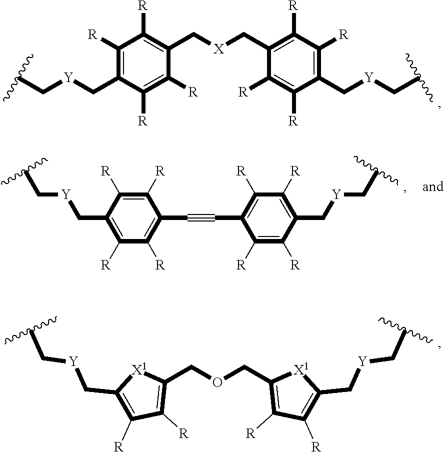

(b) the group consisting of:

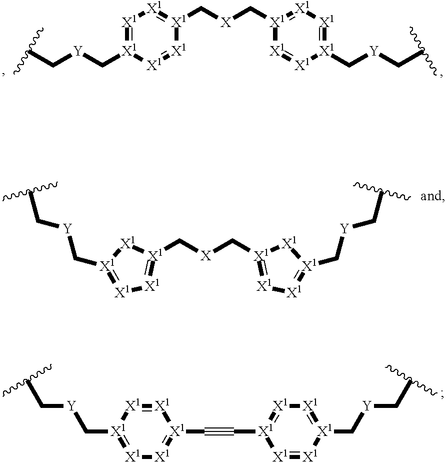

wherein:
X in group (a) and group (b) is O, S, CR₂, NR, or P (preferably O, S, CH₂ or NR);
each X¹ in group (a) is independently O, S, NH, or NR;
each X¹ in group (b) is independently O, S, C, CR, N, NH, or NR;
each R in group (a) and group (b) is independently H, alkyl, or aryl; and
each Y in group (a) and group (b) is S; and wherein

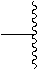

each marks a connection point to the Cα carbon in a linked residue/analogue.

As will be apparent to the skilled artisan, the antiparallel coiled-coil structures according to the present invention can each contain anywhere from only one of the linkers to all of the linkers. In at least one preferred embodiment, only one linker is present. In at least one embodiment of the antiparallel coiled-coil structures, at least one linker between a g-g′ pair or between an e-e′ pair is present and at least one linker between an a-d′ pair or between a d-a′ pair is present. Typically, the coiled-coil structures will contain the minimum number of linkers necessary to stabilize the helicity of the coiled-coil. This number will vary depending on the general stability of the unlinked coiled-coil, as will be apparent to the skilled artisan. In a preferred embodiment, only one linker is present. In a preferred embodiment, the only one linker is a linker between an e-e′ pair. In another preferred embodiment, only two linkers are present.

In at least one embodiment of the present invention, a linker between at least one of residue pairs g₀-g′₂, g₁-g′₁, g₂-g′₀, e₁-e′₃, e₂-e′₂, and e₃-e′₁ is present.

In at least one embodiment of the present invention, a linker between at least one of residue pairs a₁-d′₃, a₂-d′₂, a₃-d′₁, d₁-a′₃, d₂-a′₂, and d₃-a′₁ is present.

In at least one embodiment of the present invention, a linker between at least one

of residue pairs a₁-d′₃, a₂-d′₂, a₃-d′₁, d₁-a′₃, d₂-a′₂, and d₃-a′₁ is selected from the group consisting of disulfides, diselenides, C₁₋₈ alkylene, C₂₋₈ alkenylene, arylene, heteroarylene, triazole-diyl, and thiazole-diyl.

In at least one embodiment of the present invention, a linker between at least one of residue pairs a₁-d′₃, a₂-d′₂, a₃-d′₁, d₁-a′₃, d₂-a′₂, and d₃-a′₁ is a disulfide bond from a cysteine or homocysteine residue, a diselenide from a selenocysteine residue, an alkylene from an allylglycine residue, or an arylene linker.

In at least one embodiment, there is a linker present between X₆ and X′₁₆.

In at least one embodiment of the present invention, the antiparallel coiled-coil is of Formula III:
wherein:
each dotted line represents, independently, an optional linker and
each residue independently has the formula

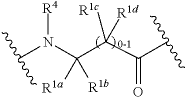

wherein:
R^{1a}, R^{1b}, R^{1c}, and R^{1d} are each independently hydrogen, an amino acid side chain, an alkyl, an alkenyl, an alkynyl, a cycloalkyl, a heterocyclyl, an aryl, a heteroaryl, or an arylalkyl, wherein each amino acid side chain, alkyl, alkenyl, alkynyl, cycloalkyl, heterocyclyl, aryl, heteroaryl, and arylalkyl can be optionally substituted with H, an alkyl, an alkenyl, an alkynyl, an azide, —OR₅, or —SR⁵; and wherein when a linker covalently binds to a residue, the linker is attached to or replaces one of R1a, R^{1b}, R^{1c}, and R^{1d};
each R⁴ is independently hydrogen, an alkyl, an alkenyl, an alkynyl, a cycloalkyl, a heterocyclyl, an aryl, a heteroaryl, or an arylalkyl; and
   each R⁵ is independently selected from the group consisting of H, -PG (where PG is a protecting group), an alkyl, an alkenyl, an alkynyl, a cycloalkyl, an aryl, a heteroaryl, a heterocyclyl, and an arylalkyl.

In at least one embodiment of the present invention, at least one of the following conditions is met: (A) in at least one a, a′, d, or d′ residue, (i) one of R^{1a} and R^{1c} is the side chain of a modified or unmodified amino acid selected from the group consisting of cysteine, homocysteine, selenocysteine, leucine, isoleucine, hexafluoroleucine, valine, hexafluorovaline, allylglycine, threonine, and analogues of each of the preceding residues, and (ii) R^{1b}, R^{1d}, and the other of R^{1a} and R^{1c} are each independently hydrogen, a C₁₋₃ alkyl, or a C₂₋₃ alkenyl; (B) in at least one e, e′, g, or g′ residue, (i) one of R^{1a} and R^{1c} is an amino acid side chain and (ii) R^{1b}, R^{1d}, and the other of R^{1a} and R^{1c} are each independently hydrogen or a C₁₋₃ alkyl.

In at least one embodiment of the present invention, the antiparallel coiled-coil has the formula:
wherein each dotted line is independently an optional linker.

In at least one embodiment of the present invention, the macrostructure is CHD-1 (e.g., CHC^{Sos}-1), CHD-2 (e.g., CHC^{Sos}-2), CHD-3 (e.g., CHC^{Sos}-3), CHD-4 (e.g., CHC^{Sos}-4), or CHD-5 (e.g., CHC^{Sos}-5). In at least one embodiment of the present invention, the macrostructure is CHD-2 (CHC^{Sos}-2) or CHD-5 (CHC^{Sos}-5).

Protecting groups function primarily to protect or mask the reactivity of functional groups. Protecting groups that are suitable for the protection of an amine group are well known in the art, including without limitation, carbamates, amides, N-alkyl and N-aryl amines, imine derivatives, enamine derivatives, and N-hetero atom derivatives as described by THEODORA W. GREENE & PETER G. M. WUTS, PROTECTIVE GROUPS IN ORGANIC SYNTHESIS 494-615 (1999), which is hereby incorporated by reference in its entirety. Suitable protecting groups according to this and all aspects of the present invention include, e.g., tert-butyloxycarbonyl (“Boc”), 9-fluorenylmethyloxycarbonyl (“Fmoc”), carbobenzyloxy (“Cbz”), and trityl. Protecting groups that are suitable for the protection of an alcohol are also well known in the art. Suitable alcohol protecting groups include, without limitation, silyl ethers, esters, and alkyl/aryl ethers. Protecting groups that are suitable for the protection of a thiol group are also well known in the art. Suitable thiol protecting groups include, without limitation, aryl/alkyl thio ethers and disulfides. As will be apparent to those of ordinary skill in the art, amino acid side chains of Asn, Asp, Gln, Glu, Cys, Ser, His, Lys, Arg, Trp, or Thr will typically need to be protected while carrying out the methods described herein. Protecting groups that are suitable for protecting these amino acid side chains are also well known in the art. Methods of protecting and deprotecting functional groups vary depending on the chosen protecting group; however, these methods are well known in the art and described in THEODORA W. GREENE & PETER G. M. WUTS, PROTECTIVE GROUPS IN ORGANIC SYNTHESIS 372-450 and 494-615 (1999), which is hereby incorporated by reference in its entirety.

A “tag” as used herein includes any labeling moiety that facilitates the detection, quantitation, separation, and/or purification of the compounds of the present invention. Suitable tags include purification tags, radioactive or fluorescent labels, and enzymatic tags.

Purification tags, such as poly-histidine (His₆-), a glutathione-S-transferase (GST-), or maltose-binding protein (MBP-), can assist in compound purification or separation but can later be removed, i.e., cleaved from the compound following recovery. Protease-specific cleavage sites can be used to facilitate the removal of the purification tag. The desired product can be purified further to remove the cleaved purification tags.

Other suitable tags include radioactive labels, such as, ¹²⁵I, ¹³¹I, ¹¹¹In, or ⁹⁹TC. Methods of radiolabeling compounds are known in the art and described in U.S. Pat. No. 5,830,431 to Srinivasan et al., which is hereby incorporated by reference in its entirety. Radioactivity is detected and quantified using a scintillation counter or autoradiography. Alternatively, the compound can be conjugated to a fluorescent tag. Suitable fluorescent tags include, without limitation, chelates (europium chelates), fluorescein and its derivatives, rhodamine and its derivatives, dansyl, Lissamine, phycoerythrin, and Texas Red. The fluorescent labels can be conjugated to the compounds using techniques disclosed in CURRENT PROTOCOLS IN IMMUNOLOGY (Coligen et al. eds., 1991), which is hereby incorporated by reference in its entirety. Fluorescence can be detected and quantified using a fluorometer.

Enzymatic tags generally catalyze a chemical alteration of a chromogenic substrate which can be measured using various techniques. For example, the enzyme may catalyze a color change in a substrate, which can be measured spectrophotometrically.

Alternatively, the enzyme may alter the fluorescence or chemiluminescence of the substrate. Examples of suitable enzymatic tags include luciferases (e.g., firefly luciferase and bacterial luciferase; see e.g., U.S. Pat. No. 4,737,456 to Weng et al., which is hereby incorporated by reference in its entirety), luciferin, 2,3-dihydrophthalazinediones, malate dehydrogenase, urease, peroxidases (e.g., horseradish peroxidase), alkaline phosphatase, β-galactosidase, glucoamylase, lysozyme, saccharide oxidases (e.g., glucose oxidase, galactose oxidase, and glucose-6-phosphate dehydrogenase), heterocyclic oxidases (e.g., uricase and xanthine oxidase), lactoperoxidase, microperoxidase, and the like. Techniques for conjugating enzymes to proteins and peptides are described in O′Sullivan et al., Methods for the Preparation of Enzyme—Antibody Conjugates for Use in Enzyme Immunoassay, in METHODS IN ENZYMOLOGY 147-66 (Langone et al. eds., 1981), which is hereby incorporated by reference in its entirety.

A targeting moiety according to the present invention functions to (i) promote the cellular uptake of the compound, (ii) target the compound to a particular cell or tissue type (e.g., signaling peptide sequence), or (iii) target the compound to a specific sub-cellular localization after cellular uptake (e.g., transport peptide sequence).

To promote the cellular uptake of a compound of the present invention, the targeting moiety may be a cell penetrating peptide (CPP). CPPs translocate across the plasma membrane of eukaryotic cells by a seemingly energy-independent pathway and have been used successfully for intracellular delivery of macromolecules, including antibodies, peptides, proteins, and nucleic acids, with molecular weights several times greater than their own. Several commonly used CPPs, including polyarginines, transportant, protamine, maurocalcine, and M918, are suitable targeting moieties for use in the present invention and are well known in the art (see Stewart et al., “Cell-Penetrating Peptides as Delivery Vehicles for Biology and Medicine,” Organic Biomolecular Chem. 6: 2242-55 (2008), which is hereby incorporated by reference in its entirety). Additionally, methods of making CPP are described in U.S. Patent Application Publication No. 20080234183 to Hallbrink et al., which is hereby incorporated by reference in its entirety.

Another suitable targeting moiety useful for enhancing the cellular uptake of a compound is an “importation competent” signal peptide as disclosed by U.S. Pat. No. 6,043,339 to Lin et al., which is hereby incorporated by reference in its entirety. An importation competent signal peptide is generally about 10 to about 50 amino acid residues in length—typically hydrophobic residues—that render the compound capable of penetrating through the cell membrane from outside the cell to the interior of the cell. An exemplary importation competent signal peptide includes the signal peptide from Kaposi fibroblast growth factor (see U.S. Pat. No. 6,043,339 to Lin et al., which is hereby incorporated by reference in its entirety). Other suitable peptide sequences can be selected from the SIGPEP database (see von Heijne G., “SIGPEP: A Sequence Database for Secretory Signal Peptides,” Protein Seq. Data Anal. 1(1): 41-42 (1987), which is hereby incorporated by reference in its entirety).

Another suitable targeting moiety is a signal peptide sequence capable of targeting the compounds of the present invention to a particular tissue or cell type. The signaling peptide can include at least a portion of a ligand binding protein. Suitable ligand binding proteins include high-affinity antibody fragments (e.g., Fab, Fab′ and F(ab′)₂, single-chain Fv antibody fragments), nanobodies or nanobody fragments, fluorobodies, or aptamers. Other ligand binding proteins include biotin-binding proteins, lipid-binding proteins, periplasmic binding proteins, lectins, serum albumins, enzymes, phosphate and sulfate binding proteins, immunophilins, metallothionein, or various other receptor proteins. For cell specific targeting, the signaling peptide is preferably a ligand binding domain of a cell specific membrane receptor. Thus, when the modified compound is delivered intravenously or otherwise introduced into blood or lymph, the compound will adsorb to the targeted cell, and the targeted cell will internalize the compound. For example, if the target cell is a cancer cell, the compound may be conjugated to an anti-C3B(I) antibody as disclosed by U.S. Pat. No. 6,572,856 to Taylor et al., which is hereby incorporated by reference in its entirety. Alternatively, the compound may be conjugated to an alphafeto protein receptor as disclosed by U.S. Pat. No. 6,514,685 to Moro, which is hereby incorporated by reference in its entirety, or to a monoclonal GAH antibody as disclosed by U.S. Pat. No. 5,837,845 to Hosokawa, which is hereby incorporated by reference in its entirety. For targeting a compound to a cardiac cell, the compound may be conjugated to an antibody recognizing elastin microfibril interfacer (EMILIN2) (Van Hoof et al., “Identification of Cell Surface for Antibody-Based Selection of Human Embryonic Stem Cell-Derived Cardiomyocytes,” J. Proteom. Res. 9: 1610-18 (2010), which is hereby incorporated by reference in its entirety), cardiac troponin I, connexin-43, or any cardiac cell-surface membrane receptor that is known in the art. For targeting a compound to a hepatic cell, the signaling peptide may include a ligand domain specific to the hepatocyte-specific asialoglycoprotein receptor. Methods of preparing such chimeric proteins and peptides are described in U.S. Pat. No. 5,817,789 to Heartlein, et al., which is hereby incorporated by reference in its entirety.

Another suitable targeting moiety is a transport peptide that directs intracellular compartmentalization of the compound once it is internalized by a target cell or tissue. For transport to the endoplasmic reticulum (ER), for example, the compound can be conjugated to an ER transport peptide sequence. A number of such signal peptides are known in the art, including the signal peptide MMSFVSLLLVGILFYATEAEQLTKCEVFQ (SEQ ID NO:31). Other suitable ER signal peptides include the N-terminus endoplasmic reticulum targeting sequence of the enzyme 17β-hydroxysteroid dehydrogenase type 11 (Horiguchi et al., “Identification and Characterization of the ER/Lipid Droplet-Targeting Sequence in 17β-hydroxysteroid Dehydrogenase Type 11,” Arch. Biochem. Biophys. 479(2): 121-30 (2008), which is hereby incorporated by reference in its entirety), or any of the ER signaling peptides (including the nucleic acid sequences encoding the ER signal peptides) disclosed in U.S. Patent Application Publication No. 20080250515 to Reed et al., which is hereby incorporated by reference in its entirety. Additionally, the compound of the present invention can contain an ER retention signal, such as the retention signal KEDL (SEQ ID NO:32). Methods of modifying the compounds of the present invention to incorporate transport peptides for localization of the compounds to the ER can be carried out as described in U.S. Patent Application Publication No. 20080250515 to Reed et al., which is hereby incorporated by reference in its entirety. For transport to the nucleus, the compounds of the present invention can include a nuclear localization transport signal. Suitable nuclear transport peptide sequences are known in the art, including the nuclear transport peptide PPKKKRKV (SEQ ID NO:33). Other nuclear localization transport signals include, for example, the nuclear localization sequence of acidic fibroblast growth factor and the nuclear localization sequence of the transcription factor NF-KB p50 as disclosed by U.S. Pat. No. 6,043,339 to Lin et al., which is hereby incorporated by reference in its entirety. Other nuclear localization peptide sequences known in the art are also suitable for use in the compounds of the present invention.

Suitable transport peptide sequences for targeting to the mitochondria include MLSLRQSIRFFKPATRTLCSSRYLL (SEQ ID NO:34). Other suitable transport peptide sequences suitable for selectively targeting the compounds of the present invention to the mitochondria are disclosed in U.S. Patent Application Publication No. 20070161544 to Wipf, which is hereby incorporated by reference in its entirety.

In some at least some embodiments of the compounds of the present invention, PG is independently selected at each occurrence thereof from the group consisting of a protecting group for protection of an amine, a protecting group for protection of a thiol, and a protecting group for protection of a carboxylic acid.

Another aspect of the present invention relates to pharmaceutical composition comprising any of the macrostructures described herein and a pharmaceutically acceptable vehicle. Acceptable pharmaceutical vehicles include solutions, suspensions, emulsions, excipients, powders, or stabilizers. The carrier should be suitable for the desired mode of delivery.

In addition, the pharmaceutical composition of the present invention may further comprise one or more pharmaceutically acceptable diluents, adjuvants, excipients, or vehicles, such as preserving agents, fillers, disintegrating agents, wetting agents, emulsifying agents, suspending agents, sweetening agents, flavoring agents, perfuming agents, antibacterial agents, antifungal agents, lubricating agents and dispensing agents, depending on the nature of the mode of administration and dosage forms. Examples of suspending agents include ethoxylated isostearyl alcohols, polyoxyethylene sorbitol and sorbitan esters, microcrystalline cellulose, aluminum metahydroxide, bentonite, agar-agar and tragacanth, or mixtures of these substances.

Prevention of the action of microorganisms can be ensured by various antibacterial and antifungal agents, for example, parabens, chlorobutanol, phenol, sorbic acid, and the like. It may also be desirable to include isotonic agents, for example sugars, sodium chloride, and the like. Prolonged absorption of the injectable pharmaceutical form can be brought about by the use of agents delaying absorption, for example, aluminum monosterate and gelatin. Examples of suitable carriers, diluents, solvents, or vehicles include water, ethanol, polyols, suitable mixtures thereof, vegetable oils (such as olive oil), and injectable organic esters such as ethyl oleate. Examples of excipients include lactose, milk sugar, sodium citrate, calcium carbonate, and dicalcium phosphate. Examples of disintegrating agents include starch, alginic acids, and certain complex silicates. Examples of lubricants include magnesium stearate, sodium lauryl sulphate, talc, as well as high molecular weight polyethylene glycols.

Yet another aspect of the present invention relates to a method of inhibiting Ras signaling in a cell. This method involves contacting the cell with a macrostructure as described herein under conditions effective to inhibit Ras signaling in the cell.

The term “inhibit” or “inhibiting” as it applies to inhibiting Ras signaling means to suppress, decrease, diminish, or lower signaling. Inhibition can be partial or complete. In at least one embodiment, inhibiting comprises preventing or delaying binding of GTP to Ras.

Suitable cells include those described infra.

In at least one embodiment, inhibiting is carried out in a subject and contacting comprises administering the compound to the subject, as described infra. Suitable subjects include those described infra.

Another aspect of the present invention is a method of treating in a subject a disorder mediated by Ras signaling. This method involves administering to the subject a macrostructure or a pharmaceutical formulation as described herein under conditions effective to treat the disorder in the subject.

A disorder meditated by Ras signaling is a disorder that is caused, at least in part, by elevated Ras activation. In at least one embodiment, elevated Ras activation is due to increased and/or prolonged binding of GTP to Ras. In at least one embodiment, elevated Ras activation is due to faster inherent conversion (i.e., conversion that does not rely on a guanosine nucleotide exchange factor) of GTP to GDP. Suitable disorders include the cellular proliferative disorders, differentiative disorders, and neoplastic conditions described infra. In at least one embodiment, the disorder is mediated by a mutated Ras protein.

The term “treatment” or “treating” means any manner in which one or more

symptoms of a disease or disorder are ameliorated or otherwise beneficially altered. Treatment also encompasses any pharmaceutical use of the compositions herein.

Another aspect of the present invention relates to a method of using a macrostructure as described herein to treat cancers and neoplastic conditions. As used herein, the terms “cancer”, “hyperproliferative”, and “neoplastic” refer to cells having the capacity for autonomous growth, i.e., an abnormal state or condition characterized by rapidly proliferating cell growth. Hyperproliferative and neoplastic disease states may be categorized as pathologic, i.e., characterizing or constituting a disease state, or may be categorized as non-pathologic, i.e., a deviation from normal but not associated with a disease state. The term is meant to include all types of cancerous growths or oncogenic processes, metastatic tissues, or malignantly transformed cells, tissues, or organs, irrespective of histopathologic type or stage of invasiveness. A metastatic tumor can arise from a multitude of primary tumor types, including but not limited to those of breast, lung, liver, colon and ovarian origin. “Pathologic hyperproliferative” cells occur in disease states characterized by malignant tumor growth. Examples of non-pathologic hyperproliferative cells include proliferation of cells associated with wound repair. Examples of cellular proliferative and/or differentiative disorders include cancer, e.g., carcinoma, sarcoma, or metastatic disorders. In some embodiments, the compounds are novel therapeutic agents for controlling breast cancer, ovarian cancer, colon cancer, pancreatic cancer, bladder cancer, lung cancer, metastasis of such cancers, and the like.

Suitable subjects include those described infra.

Examples of cancers or neoplastic conditions include, but are not limited to, a fibrosarcoma, myosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, gastric cancer, esophageal cancer, rectal cancer (incl. rectal adenocarcinoma), pancreatic cancer, endometrium carcinoma, salivary gland carcinoma, mouth carcinoma, ovarian cancer, ovarian carcinoma, prostate cancer, uterine cancer, cancer of the head and neck, skin cancer, brain cancer, squamous cell carcinoma, sebaceous gland carcinoma, papillary carcinoma, papillary adenocarcinoma, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular cancer, small cell lung carcinoma, non-small cell lung carcinoma, lung adenocarcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma, leukemia, lymphoma, and Kaposi sarcoma.

Examples of proliferative disorders include hematopoietic neoplastic disorders. As used herein, the term “hematopoietic neoplastic disorders” includes diseases involving hyperplastic/neoplastic cells of hematopoietic origin, e.g., arising from myeloid, lymphoid or erythroid lineages, or precursor cells thereof. Preferably, the diseases arise from poorly differentiated acute leukemias, e.g., erythroblastic leukemia and acute megakaryoblastic leukemia. Additional exemplary myeloid disorders include, but are not limited to, plasma cell myeloma, acute myeloid leukemia (AML), acute promyeloid leukemia (APML), acute myelogenous leukemia (AL) and chronic myelogenous leukemia (CL) (reviewed in Vaickus et al., “Immune Markers in Hematologic Malignancies,” Crit. Rev. Oncol. Hemotol. 11: 267-97 (1991), which is hereby incorporated by reference in its entirety). Lymphoid malignancies include, but are not limited to acute lymphoblastic leukemia (ALL) which includes B-lineage ALL and T-lineage ALL, chronic lymphocytic leukemia (CLL), pro lymphocytic leukemia (PLL), hairy cell leukemia (HLL), and Waldenstrom's macroglobulinemia (WM). Additional forms of malignant lymphomas include, but are not limited to non-Hodgkin lymphoma and variants thereof, peripheral T cell lymphomas, adult T cell leukemia/lymphoma (ATL), cutaneous T-cell lymphoma (CTCL), large granular lymphocytic leukemia (LGF), Hodgkin's disease, and Reed-Stemberg disease. Additional hematopoietic neoplastic disorders include chronic myelomonocytic leukemia (CMML).

Examples of cellular proliferative and/or differentiative disorders of the breast include, but are not limited to, proliferative breast disease including, e.g., epithelial hyperplasia, sclerosing adenosis, and small duct papillomas; tumors, e.g., stromal tumors such as fibroadenoma, phyllodes tumor, and sarcomas, and epithelial tumors such as large duct papilloma; carcinoma of the breast including in situ (noninvasive) carcinoma that includes ductal carcinoma in situ (including Paget's disease) and lobular carcinoma in situ, and invasive (infiltrating) carcinoma including, but not limited to, invasive ductal carcinoma, invasive lobular carcinoma, medullary carcinoma, colloid (mucinous) carcinoma, tubular carcinoma, and invasive papillary carcinoma, and miscellaneous malignant neoplasms. Disorders in the male breast include, but are not limited to, gynecomastia and carcinoma.

Examples of cellular proliferative and/or differentiative disorders of the lung include, but are not limited to, lung adenocarcinoma, bronchogenic carcinoma, including paraneoplastic syndromes, bronchioloalveolar carcinoma, neuroendocrine tumors, such as bronchial carcinoid, miscellaneous tumors, and metastatic tumors; pathologies of the pleura, including inflammatory pleural effusions, noninflammatory pleural effusions, pneumothorax, and pleural tumors, including solitary fibrous tumors (pleural fibroma) and malignant mesothelioma.

Examples of cellular proliferative and/or differentiative disorders of the colon include, but are not limited to, non-neoplastic polyps, adenomas, familial syndromes, colorectal carcinogenesis, colorectal carcinoma, and carcinoid tumors.

Examples of cellular proliferative and/or differentiative disorders of the liver include, but are not limited to, nodular hyperplasias, adenomas, and malignant tumors, including primary carcinoma of the liver and metastatic rumors.

Examples of cellular proliferative and/or differentiative disorders of the ovary include, but are not limited to, ovarian tumors such as, tumors of coelomic epithelium, serous tumors, mucinous tumors, endometrioid tumors, clear cell adenocarcinoma, cystadenofibroma, Brenner tumor, surface epithelial tumors; germ cell tumors such as mature (benign) teratomas, monodermal teratomas, immature malignant teratomas, dysgerminoma, endodermal sinus tumor, choriocarcinoma; sex cord-stomal tumors such as, granulosa-theca cell tumors, thecomafibromas, androblastomas, hill cell tumors, and gonadoblastoma; and metastatic tumors such as Krukenberg tumors.

In some embodiments, the compounds described herein are used to treat a cancer mediated by a mutated Ras protein. Cancers known to frequently involve such mutations include, but are not limited to, non-small-cell lung cancer (adenocarcinoma), colorectal cancer, pancreatic cancer, thyroid cancers (e.g. , follicular, undifferentiated papillary or papillary; e.g., thyroid carcinoma), seminoma, melanoma, bladder cancer, liver cancer, kidney cancer, myelodysplastic syndrome, and acute myelogenous leukemia.

### Breast Cancer

In some embodiments, the invention provides methods of treating breast cancer by administering the compounds described herein. Breast cancer includes invasive breast carcinomas, such as invasive ductal carcinoma, invasive lobular carcinoma, tubular carcinoma, invasive cribriform carcinoma, medullary carcinoma, mucinous carcinoma and other tumors with abundant mucin, cystadenocarcinoma, columnar cell mucinous carcinoma, signet ring cell carcinoma, neuroendocrine tumors (including solid neuroendocrine carcinoma, atypical carcinoid tumour, small cell/oat cell carcinoma, or large cell neuroendocrine carcinoma), invasive papillary carcinoma, invasive micropapillary carcinoma, apocrine carcinoma, metaplastic carcinomas, pure epithelial metaplastic carcinomas, mixed epithelial/mesenchymal metaplastic carcinomas, lipid-rich carcinoma, secretory carcinoma, oncocytic carcinoma, adenoid cystic carcinoma, acinic cell carcinoma, glycogen-rich clear cell carcinoma, sebaceous carcinoma, inflammatory carcinoma or bilateral breast carcinoma; mesenchymal tumors such as haemangioma, angiomatosis, haemangiopericytoma, pseudoangiomatous stromal hyperplasia, myofibroblastoma, fibromatosis (aggressive), inflammatory myofibroblast tumour, lipoma, angiolipoma, granular cell tumour, neurofibroma, schwannoma, angiosarcoma, liposarcoma, rhabdomyosarcoma, osteosarcoma, leiomyoma, or leiomysarcoma; myoepithelial lesions such as myoepitheliosis, adenomyo epithelial adenosis, adenomyoepithelioma, or malignant myoepithelioma; fibroepithelial tumors such as fibroadenoma, phyllodes tumour, low grade periductal stromal sarcoma, or mammary hamartoma; and tumors of the nipple such as nipple adenoma, syringomatous adenoma, or Paget's disease of the nipple.

Treatment of breast cancer may be effected in conjunction with any additional therapy, such as a therapy that is part of the standard of care. A surgical technique such as lumpectomy or mastectomy may be performed prior to, during, or following treatment with the compounds described herein. Alternatively, radiation therapy may be used for the treatment of breast cancer in conjunction with the compounds described herein. In other cases, the compounds described herein are administered in combination with a second therapeutic agent. Such an agent may be a chemotherapeutic agent such as an individual drug or combination of drugs and therapies. For example, the chemotherapeutic agent can be an adjuvant chemotherapeutic treatment such as CMF (cyclophosphamide, methotrexate, and 5-fluorouracil); FAC or CAF (5-fluorouracil, doxorubicin, cyclophosphamide); AC or CA (doxorubicin and cyclophosphamide); AC-Taxol (AC followed by paclitaxel); TAC (docetaxel, doxorubicin, and cyclophosphamide); FEC (5-fluorouracil, epirubicin, and cyclophosphamide); FECD (FEC followed by docetaxel); TC (docetaxel and cyclophosphamide). In addition to chemotherapy, trastuzumab may also be added to the regimen depending on the tumor characteristics (i.e., HER2/neu status) and risk of relapse. Hormonal therapy may also be appropriate before, during or following chemotherapeutic treatment. For example, tamoxifen may be administered or a compound in the category of aromatase inhibitors including, but not limited to aminogluthetimide, anastrozole, exemestane, formestane, letrozole, or vorozole. In other embodiments, an antiangiogenic agent may be used in combination therapy for the treatment of breast cancer. The antiangiogenic agent may be an anti-VEGF agent including, but not limited to bevacizumab.

### Ovarian Cancer

In some embodiments, the compounds described herein may be used to treat ovarian cancer. Ovarian cancers include ovarian tumors such as, tumors of coelomic epithelium, serous tumors, mucinous tumors, endometrioid tumors, clear cell adenocarcinoma, cystadenofibroma, Brenner tumor, surface epithelial tumors; germ cell tumors such as mature (benign) teratomas, monodermal teratomas, immature malignant teratomas, dysgerminoma, endodermal sinus tumor, choriocarcinoma; sex cord-stomal tumors such as, granulosa-theca cell tumors, thecomafibromas, androblastomas, hill cell tumors, and gonadoblastoma; and metastatic tumors such as ukenberg tumors.

The compounds of the present invention may be administered in conjunction with a second therapy such as a therapy that is part of the standard of care. Surgery, immunotherapy, chemotherapy, hormone therapy, radiation therapy, or a combination thereof are some possible treatments available for ovarian cancer. Some possible surgical procedures include debulking, and a unilateral or bilateral oophorectomy and/or a unilateral or bilateral salpingectomy.

Anti-cancer drugs that may be used include cyclophosphamide, etoposide, altretamine, and ifosfamide. Hormone therapy with the drug tamoxifen may be used to shrink ovarian tumors. Radiation therapy may be external beam radiation therapy and/or brachytherapy.

### Prostate Cancer

In some embodiments, the compounds described herein may be used to treat prostate cancer. Prostate cancers include adenocarcinomas and metastasized adenocarcinomas. The compounds described herein may be administered in conjunction with a second therapy such as a therapy that is part of the standard of care. Treatment for prostate cancer may involve surgery, radiation therapy, High Intensity Focused Ultrasound (HIFU), chemotherapy, cryosurgery, hormonal therapy, or any combination thereof. Surgery may involve prostatectomy, radical perineal prostatectomy, laparoscopic radical prostatectomy, transurethral resection of the prostate or orchiectomy. Radiation therapy may include external beam radiation therapy and/or brachytherapy. Hormonal therapy may include orchiectomy, administration of antiandrogens such as flutamide, bicalutamide, nilutamide, or cyproterone acetate; medications which inhibit the production of adrenal androgens such as DHEA, such as ketoconazole and aminoglutethimide; and GnRH antagonists or agonists such as Abarelix (Plenaxis®), Cetrorelix (Cetrotide®), Ganirelix (Antagon®), leuprolide, goserelin, triptorelin, or buserelin. Treatment with an anti-androgen agent, which blocks androgen activity in the body, is another available therapy. Such agents include flutamide, bicalutamide, and nilutamide. This therapy is typically combined with LHRH analog administration or an orchiectomy, which is termed a combined androgen blockade (CAB). Chemotherapy includes, but is not limited to, administration of docetaxel, for example with a corticosteroid such as prednisone. Anti-cancer drugs such as doxorubicin, estramustine, etoposide, mitoxantrone, vinblastine, paclitaxel, carboplatin may also be administered to slow the growth of prostate cancer, reduce symptoms and improve the quality of life. Additional compounds such as bisphosphonate drugs may also be administered.

### Renal Cancer

In some embodiments, the compounds described herein may be used to treat renal cancer. Renal cancers include, but are not limited to, renal cell carcinomas, metastases from extra-renal primary neoplasms, renal lymphomas, squamous cell carcinomas, juxtaglomerular tumors (reninomas), transitional cell carcinomas, angiomyolipomas, oncocytomas and Wilm's tumors. The compounds described herein may be administered in conjunction with a second therapy such as a therapy that is part of the standard of care. Treatment for renal cancer may involve surgery, percutaneous therapies, radiation therapies, chemotherapy, vaccines, or other medication. Surgical techniques useful for treatment of renal cancer in combination with the compounds described herein include nephrectomy, which may include removal of the adrenal gland, retroperitoneal lymph nodes, and any other surrounding tissues affected by the invasion of the tumor. Percutaneous therapies include, for example, image-guided therapies which may involve imaging of a tumor followed by its targeted destruction by radiofrequency ablation or cryotherapy. In some cases, other chemotherapeutic or other medications useful in treating renal cancer may be alpha-interferon, interleukin-2, bevacizumab, sorafenib, sunitib, temsirolimus or other kinase inhibitors.

### Pancreatic Cancer

In some embodiments, the invention provides methods of treating pancreatic cancer by administering compounds described herein, such as a pancreatic cancer selected from the following: an epitheliod carcinoma in the pancreatic duct tissue and an adenocarcinoma in a pancreatic duct. The most common type of pancreatic cancer is an adenocarcinoma, which occurs in the lining of the pancreatic duct. Possible treatments available for pancreatic cancer include surgery, immunotherapy, radiation therapy, and chemotherapy. Possible surgical treatment options include a distal or total pancreatectomy and a pancreaticoduodenectomy (Whipple procedure). Radiation therapy may be an option for pancreatic cancer patients, specifically external beam radiation where radiation is focused on the tumor by a machine outside the body. Another option is intraoperative electron beam radiation administered during an operation.

Chemotherapy may also be used to treat pancreatic cancer patients. Suitable anticancer drugs include, but are not limited to, 5-fluorouracil (5-FU), mitomycin, ifosfamide, doxorubicin, streptozocin, chlorozotocin, and combinations thereof. The methods provided by the invention can provide a beneficial effect for pancreatic cancer patients, by administration of a polypeptide of the invention or a combination of administration of a compound and surgery, radiation therapy, or chemotherapy.

### Colon Cancer

In some embodiments, compounds described herein may be used for the treatment of colon cancer, including but not limited to non-neoplastic polyps, adenomas, familial syndromes, colorectal carcinogenesis, colorectal carcinoma, colon adenocarcinoma, and carcinoid tumors. Possible treatments available for colon cancer that may be used in conjunction with the compounds described herein include surgery, chemotherapy, radiation therapy, or targeted drug therapy.

Radiation therapy may include external beam radiation therapy and/or brachytherapy.

Chemotherapy may be used to reduce the likelihood of metastasis developing, shrink tumor size, or slow tumor growth. Chemotherapy is often applied after surgery (adjuvant), before surgery (neo-adjuvant), or as the primary therapy if surgery is not indicated (palliative). For example, exemplary regimens for adjuvant chemotherapy involve the combination of infusional 5-fluorouracil, leucovorin, and oxaliplatin (FOLFOX). First line chemotherapy regimens may involve the combination of infusional 5-fluorouracil, leucovorin, and oxaliplatin (FOLFOX) with a targeted drug such as bevacizximab, cetuximab or panitumumab or infusional 5-fluorouracil, leucovorin, and irinotecan (FOLFIRI) with targeted drug such as bevacizximab, cetuximab or panitumumab. Other chemotherapeutic agents that may be useful in the treatment of colon cancer in combination with the compounds described herein are Bortezomib (Velcade®), Oblimersen (Genasense®, G3139), Gefitinib and Erlotinib (Tarceva®) and Topotecan (Hycamtin®).

### Lung Cancer

Some embodiments provide methods for the treatment of lung cancer using the compounds described herein. Examples of cellular proliferative and/or differentiative disorders of the lung include, but are not limited to, bronchogenic carcinoma, including paraneoplastic syndromes, bronchioloalveolar carcinoma, neuroendocrine tumors, such as bronchial carcinoid, miscellaneous tumors, and metastatic tumors; pathologies of the pleura, including inflammatory pleural effusions, noninflammatory pleural effusions, pneumothorax, and pleural tumors, including solitary fibrous tumors (pleural fibroma) and malignant mesothelioma.

The most common type of lung cancer is non-small cell lung cancer (SCLC), which accounts for approximately 80-85% of lung cancers and is divided into squamous cell carcinomas, adenocarcinomas, and large cell undifferentiated carcinomas. Small cell lung cancer, e.g. , small cell lung carcinomas, accounts for 15-20% of lung cancers. Treatment options for lung cancer include surgery, immunotherapy, radiation therapy, chemotherapy, photodynamic therapy, or a combination thereof. Some possible surgical options for treatment of lung cancer are a segmental or wedge resection, a lobectomy, or a pneumonectomy. Radiation therapy may be external beam radiation therapy or brachytherapy. Some anti-cancer drugs that may be used in chemotherapy to treat lung cancer in combination with the compounds described herein include cisplatin, carboplatin, paclitaxel, docetaxel, gemcitabine, vinorelbine, irinotecan, etoposide, vinblastine, gefitinib, ifosfamide, methotrexate, or a combination thereof. Photodynamic therapy (PDT) may be used to treat lung cancer patients. The methods described herein can provide a beneficial effect for lung cancer patients, by administration of a compound or a combination of administration of a compound and surgery, radiation therapy, chemotherapy, photodynamic therapy, or a combination thereof.

### Liver Disorders

Examples of cellular proliferative and/or differentiative disorders of the liver include, but are not limited to, nodular hyperplasias, adenomas, and malignant tumors, including primary carcinoma of the liver and metastatic tumors.

### Immunoproliferative Disorders

Immunoproliferative disorders (also known as “immunoproliferative diseases” or “immunoproliferative neoplasms”) are disorders of the immune system that are characterized by the abnormal proliferation of the primary cells of the immune system, which includes B cells, T cells, and Natural Killer (K) cells, or by the excessive production of immunoglobulins (also known as antibodies). Such disorders include the general categories of lymphoproliferative disorders, hypergammaglobulinemias, and paraproteinemias. Examples of such disorders include, but are not limited to, X-linked lymphoproliferative disorder, autosomal lymphoproliferative disorder, Hyper-Ig syndrome, heavy chain disease, and cryoglobulinemia. Other immunoproliferative disorders can be graft versus host disease (GVHD); psoriasis; immune disorders associated with graft transplantation rejection; T cell lymphoma; T cell acute lymphoblastic leukemia; testicular angiocentric T cell lymphoma; benign lymphocytic angiitis; and autoimmune diseases such as lupus erythematosus, Hashimoto's thyroiditis, primary myxedema, Graves' disease, pernicious anemia, autoimmune atrophic gastritis, Addison's disease, insulin dependent diabetes mellitis, good pasture's syndrome, myasthenia gravis, pemphigus, Crohn's disease, sympathetic ophthalmia, autoimmune uveitis, multiple sclerosis, autoimmune hemolytic anemia, idiopathic thrombocytopenia, primary biliary cirrhosis, chronic action hepatitis, ulceratis colitis, Sjogren's syndrome, rheumatoid arthritis, polymyositis, scleroderma, and mixed connective tissue disease.

### Combination Treatments

In one embodiment, compounds described herein may be used for the treatment of cancer in conjunction with alkylating and alkylating-like agents. Such agents include, for example, nitrogen mustards such as chlorambucil, chlormethme, cyclophosphamide, ifosfamide, and melphalan; nitrosoureas such as carmustine, fotemustine, lomustine, and streptozocin; platinum therapeutic agents such as carboplatin, cisplatin, oxaliplatin, BBR3464, and satraplatin; or other agents, including but not limited to busulfan, dacarbazine, procarbazine, temozolomide, thiotepa, treosulfan, or uramustine.

In another embodiment, compounds described herein may be used in conjunction with an antineoplastic agent which is an antimetabolite. For example, such an antineoplastic agent may be a folic acid such as aminopterin, methotrexate, pemetrexed, or raltitrexed. Alternatively, the antineoplastic agent may be a purine, including but not limited to cladribine, clofarabine, fludarabine, mercaptopurine, pentostatin, thioguanine. In further embodiments, the antineoplastic agent may be a pyrimidine such as capecitabine, cytarabine, fluorouracil, floxuridine, and gemcitabine.

In still other embodiments, compounds described herein may be used in conjunction with an antineoplastic agent which is an spindle poison/mitotic inhibitor. Agents in this category include taxanes, for example docetaxel and paclitaxel; and vinca alkaloids such as vinblastine, vincristine, vindesine, and vinorelbine. In yet other embodiments, compounds described herein may be used in combination with an antineoplastic agent which is a cytotoxic/antitumor antibiotic from the anthracycline family such as daunorubicin, doxorubicin, epirubicin, idarubicin, mitoxantrone, pixantrone, or valrubicin; an antibiotic from the streptomyces family such as actinomycin, bleomycin, mitomycin, or plicamycin; or hydroxyurea. Alternatively, agents used for combination therapy may be topoisomerase inhibitors including, but not limited to camptothecin, topotecan, irinotecan, etoposide, or teniposide.

Alternatively, the antineoplastic agent may be an antibody or antibody-derived agent. For example, a receptor tyrosine kinase-targeted antibody such as cetuximab, panitumumab, or trastuzumab may be used. Alternatively, the antibody may be an anti-CD20 antibody such as rituximab or tositumomab, or any other suitable antibody including but not limited to alemtuzumab, bevacizumab, and gemtuzumab. In other embodiments, the antineoplastic agent is a photosensitizer such as aminolevulinic acid, methyl aminolevulinate, porfimer sodium, or verteporfm. In still other embodiments, the antineoplastic agent is a tyrosine kinase inhibitor such as dediranib, dasatinib, erlotinib, gefitinib, imatinib, lapatinib, nilotinib, sorafenib, sunitinib, or vandetanib. Other neoplastic agents suitable in the use of the invention include, for example, alitretinoin, tretinoin, altretamine, amsacrine, anagrelide, arsenic trioxide, asparaginase (pegaspargase), bexarotene, bortezomib, denileukin diftitox, estramustine, ixabepilone, masoprocol, or mitotane.

In other or further embodiments, the compounds described herein are used to treat conditions characterized by overactive cell death or cellular death due to physiologic insult, etc. Some examples of conditions characterized by premature or unwanted cell death are or alternatively unwanted or excessive cellular proliferation include, but are not limited to hypocellular hypoplastic, acellular/aplastic, or hypercellular/hyperplastic conditions. Some examples include hematologic disorders including but not limited to fanconi anemia, aplastic anemia, thalassemia, congenital neutropenia, and myelodysplasia.

In other or further embodiments, the compounds described herein that act to decrease apoptosis are used to treat disorders associated with an undesirable level of cell death. Thus, in some embodiments, the anti-apoptotic compounds described herein are used to treat disorders such as those that lead to cell death associated with viral infection, e.g., infection associated with infection with human immunodeficiency virus (HIV). A wide variety of neurological diseases are characterized by the gradual loss of specific sets of neurons, and the anti-apoptotic compounds described herein are used, in some embodiments, in the treatment of these disorders. Such disorders include Alzheimer's disease, Parkinson's disease, amyotrophic lateral sclerosis (ALS) retinitis pigmentosa, spinal muscular atrophy, and various forms of cerebellar degeneration. The cell loss in these diseases does not induce an inflammatory response, and apoptosis appears to be the mechanism of cell death. In addition, a number of hematologic diseases are associated with a decreased production of blood cells. These disorders include anemia associated with chronic disease, aplastic anemia, chronic neutropenia, and the myelodysplastic syndromes. Disorders of blood cell production, such as myelodysplasia syndrome and some forms of aplastic anemia, are associated with increased apoptotic cell death within the bone marrow. These disorders could result from the activation of genes that promote apoptosis, acquired deficiencies in stromal cells or hematopoietic survival factors, or the direct effects of toxins and mediators of immune responses. Two common disorders associated with cell death are myocardial infarctions and stroke. In both disorders, cells within the central area of ischemia, which is produced in the event of acute loss of blood flow, appear to die rapidly as a result of necrosis. However, outside the central ischemic zone, cells die over a more protracted time period and morphologically appear to die by apoptosis.

### Administering/Contacting/Cells/Subjects

In all aspects of the present invention involving administering a macrostructure described herein, the compounds can be administered orally, parenterally, for example, subcutaneously, intravenously, intramuscularly, intraperitoneally, by intranasal instillation, or by application to mucous membranes, such as, that of the nose, throat, and bronchial tubes. They may be administered alone or with suitable pharmaceutical carriers, and can be in solid or liquid form such as, tablets, capsules, powders, solutions, suspensions, or emulsions.

The active compounds of the present invention may be orally administered, for example, with an inert diluent, or with an assimilable edible carrier, or they may be enclosed in hard or soft shell capsules, or they may be compressed into tablets, or they may be incorporated directly with the food of the diet. For oral therapeutic administration, these active compounds may be incorporated with excipients and used in the form of tablets, capsules, elixirs, suspensions, syrups, and the like. Such compositions and preparations should contain at least 0.1% of active compound. The percentage of the compound in these compositions may, of course, be varied and may conveniently be between about 2% to about 60% of the weight of the unit. The amount of active compound in such therapeutically useful compositions is such that a suitable dosage will be obtained. Preferred compositions according to the present invention are prepared so that an oral dosage unit contains between about 1 and 250 mg of active compound.

The tablets, capsules, and the like may also contain a binder such as gum tragacanth, acacia, corn starch, or gelatin; excipients such as dicalcium phosphate; a disintegrating agent such as corn starch, potato starch, alginic acid; a lubricant such as magnesium stearate; and a sweetening agent such as sucrose, lactose, or saccharin. When the dosage unit form is a capsule, it may contain, in addition to materials of the above type, a liquid carrier, such as a fatty oil.

Various other materials may be present as coatings or to modify the physical form of the dosage unit. For instance, tablets may be coated with shellac, sugar, or both. A syrup may contain, in addition to active ingredient, sucrose as a sweetening agent, methyl and propylparabens as preservatives, a dye, and flavoring such as cherry or orange flavor.

These active compounds may also be administered parenterally. Solutions or suspensions of these active compounds can be prepared in water suitably mixed with a surfactant, such as hydroxypropylcellulose. Dispersions can also be prepared in glycerol, liquid polyethylene glycols, and mixtures thereof in oils. Illustrative oils are those of petroleum, animal, vegetable, or synthetic origin, for example, peanut oil, soybean oil, or mineral oil. In general, water, saline, aqueous dextrose and related sugar solution, and glycols such as, propylene glycol or polyethylene glycol, are preferred liquid carriers, particularly for injectable solutions. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol), suitable mixtures thereof, and vegetable oils.

The compounds of the present invention may also be administered directly to the airways in the form of an aerosol. For use as aerosols, the compounds of the present invention in solution or suspension may be packaged in a pressurized aerosol container together with suitable propellants, for example, hydrocarbon propellants like propane, butane, or isobutane with conventional adjuvants. The materials of the present invention also may be administered in a non-pressurized form such as in a nebulizer or atomizer.

In all aspects of the present invention involving contacting a cell, suitable cells include, without limitation, mammalian cells (e.g., primate cells including human cells, cat cells, dog cells, horse cells, cattle cells, goat cells, sheep cells, pig cells, mice cells, rat cells). In at least one preferred embodiment, the cells express a mutated Ras protein.

In all aspects of the present invention involving a subject, suitable subjects include mammals (e.g., primates such as humans, cats, dogs, horses, cattle, goats, sheeps, pigs, mice, rats). In at least one preferred embodiment, the subject has a disorder mediated by a mutated Ras protein.

In all methods involving mutated Ras proteins, suitable examples include H-Ras

isoforms, N-Ras isoforms, and K-Ras isoforms. In at least one embodiment, the mutated Ras protein is an H-Ras isoform. In at least one embodiment, the mutated Ras protein is a K-Ras isoform. In at least one embodiment, the mutated Ras protein has one or more mutations selected from the group consisting of G12C, G12D, G12S, G12V, G13D, G12A, G12C, G12D, G12R, G12S, G12V, G13A, G13C, G13R, G13S, G13D, Q61E, Q61H, Q61L, Q61K, Q61P, and Q61R. In at least one preferred embodiment, the mutated Ras protein has a G12C or G12D mutation.

In all aspects of the present invention directed to methods involving contacting a cell with one or more compounds, contacting can be carried out using methods that will be apparent to the skilled artisan, and can be done in vitro or in vivo. In at least one embodiment of the present invention, contacting stabilizes Ras in an inactive state.

One approach for delivering agents into cells involves the use of liposomes. Basically, this involves providing a liposome which includes agent(s) to be delivered, and then contacting the target cell, tissue, or organ with the liposomes under conditions effective for delivery of the agent into the cell, tissue, or organ.

This liposome delivery system can also be made to accumulate at a target organ, tissue, or cell via active targeting (e.g., by incorporating an antibody or hormone on the surface of the liposomal vehicle). This can be achieved according to known methods.

An alternative approach for delivery of protein- or polypeptide-containing agents involves the conjugation of the desired agent to a polymer that is stabilized to avoid enzymatic degradation of the conjugated protein or polypeptide. Conjugated proteins or polypeptides of this type are described in U.S. Pat. No. 5,681,811 to Ekwuribe, which is hereby incorporated by reference in its entirety.

Yet another approach for delivery of agents involves preparation of chimeric agents according to U.S. Pat. No. 5,817,789 to Heartlein et al., which is hereby incorporated by reference in its entirety. The chimeric agent can include a ligand domain and the agent (e.g., a compound of the invention). The ligand domain is specific for receptors located on a target cell. Thus, when the chimeric agent is delivered intravenously or otherwise introduced into blood or lymph, the chimeric agent will adsorb to the targeted cell, and the targeted cell will internalize the chimeric agent.

Compounds of the present invention may be delivered directly to the targeted cell/tissue/organ.

Additionally and/or alternatively, the compounds may be administered to a non-targeted area along with one or more agents that facilitate migration of the compounds to (and/or uptake by) a targeted tissue, organ, or cell. As will be apparent to one of ordinary skill in the art, the compound itself can be modified to facilitate its transport to a target tissue, organ, or cell and/or to facilitate its uptake by a target cell (e.g., its transport across cell membranes).

These aspects of the present technology are further illustrated by the following examples. All references cited throughout this application, including in the drawings and

Examples, are hereby incorporated by reference in their entirety.

### EXAMPLES

The following examples are provided to illustrate embodiments of the present technology, but they are by no means intended to limit its scope.

Solubility was one of the most difficult challenges to overcome and was critical for carrying out the majority of the experiments described herein. Judicious placement of charged and polar residues was required to enhance solubility under aqueous conditions without sacrificing affinity.

### Example 1: Synthesis of Crosslinked Helix Dimer (CHD) Peptides

Peptide synthesis was carried out under standard Fmoc solid-phase peptide synthesis on Rink Amide resin unless otherwise specified. The completed peptides undergo a final Fmoc deprotection and are protected with an acetyl group at the N-terminus using a solution of 0.5 M acetic anhydride and 5% diisopropylethylamine (DIEA) in NMP for 30 minutes prior to cleavage from resin. The two desired peptide strands were cleaved from resin, purified via RP-HPLC, and characterized by MALDI-TOF spectrometry. The two strands are crosslinked as previously described and shown below in Scheme 1 (J. Sadek et al., Modulation of virus-induced NF-kappaB signaling by NEMO coiled coil mimics. Nat Commun 11, 1786 (2020); M. G. Wuo, S. H. Hong, A. Singh, P. S. Arora, Synthetic Control of Tertiary Helical Structures in Short Peptides. J. Am. Chem. Soc. 140, 16284-16290 (2018); M. G. Wuo, A. B. Mahon, P. S. Arora, An Effective Strategy for Stabilizing Minimal Coiled Coil Mimetics. J. Am. Chem. Soc. 137, 11618-11621 (2015)).

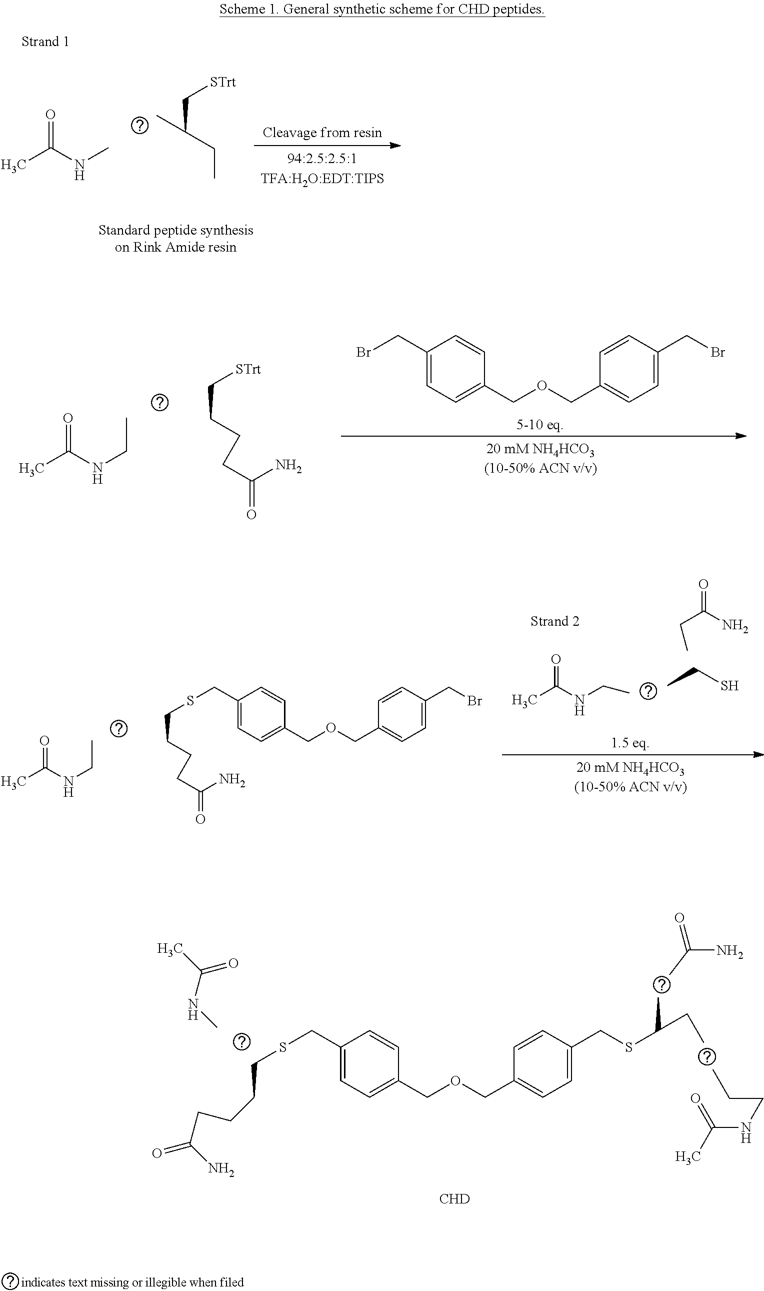

Each strand contains a single cysteine residue strategically placed for addition of the dibenzylether crosslinker. The crosslinker was synthesized according to previously published protocols (M. G. Wuo, S. H. Hong, A. Singh, P. S. Arora, Synthetic Control of Tertiary Helical Structures in Short Peptides. J. Am. Chem. Soc. 140, 16284-16290 (2018)). A purified strand was initially dissolved in 20 mM NH₄CO₃ buffer (pH=8.1) and subsequently added to a solution of the dibenzylether crosslinker (5-10 eq.) in acetonitrile. The reaction mixture was stirred for 90 minutes at 20° C. followed by purification via RP-HPLC (25-65% acetonitrile gradient in water with 0.1% TFA over 40 minutes) on a C₁₈ reverse-phase column. The purified product was subsequently lyophilized. The lyophilized mono-cysteine alkylated peptide was then dissolved in 1:1 acetonitrile:20 mM aqueous NH₄CO₃ solution (pH 8.1). An excess amount of the second strand with a free cysteine (1.5 eq.) was added, and the resulting mixture was stirred for 1 hour at 20° C. The reaction mixture was subjected to RP-HPLC (25-65% acetonitrile gradient in water with 0.1% TFA over 40 minutes) on a C₁₈ reverse-phase column) and characterized by analytical HPLC and MALDI-TOF spectroscopy.

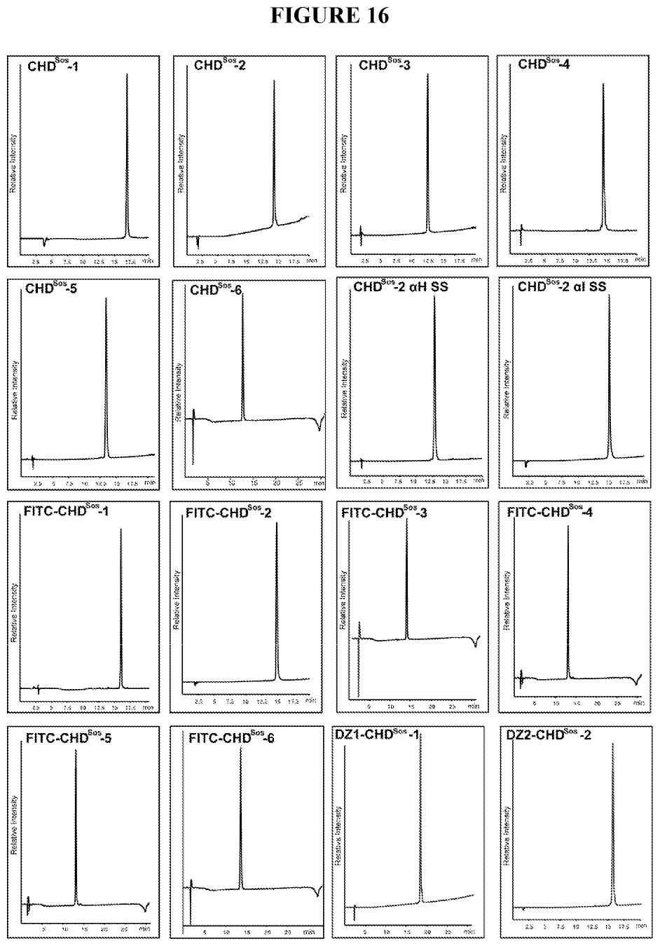

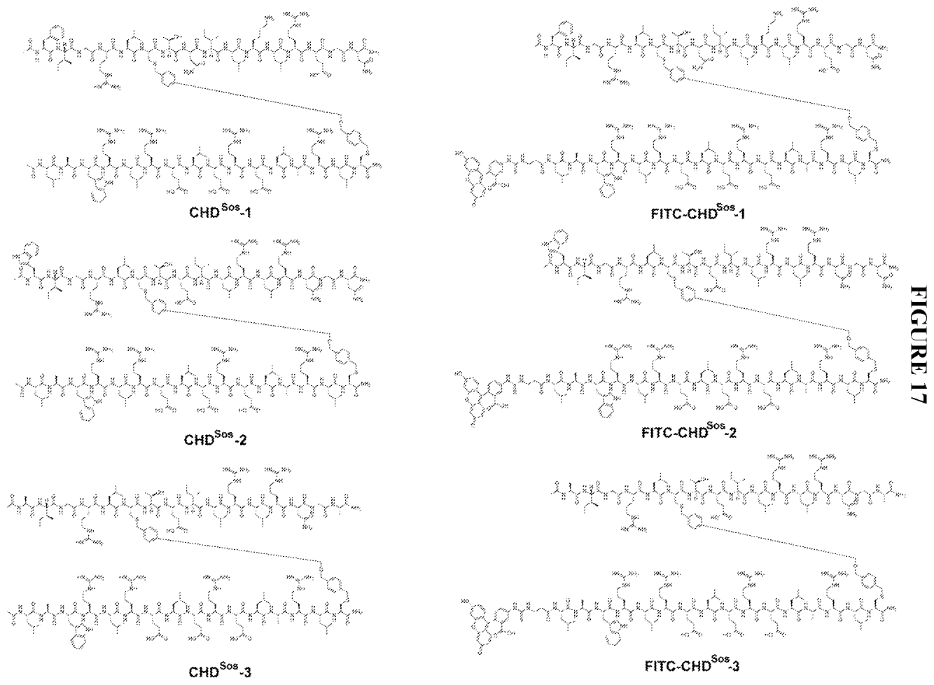

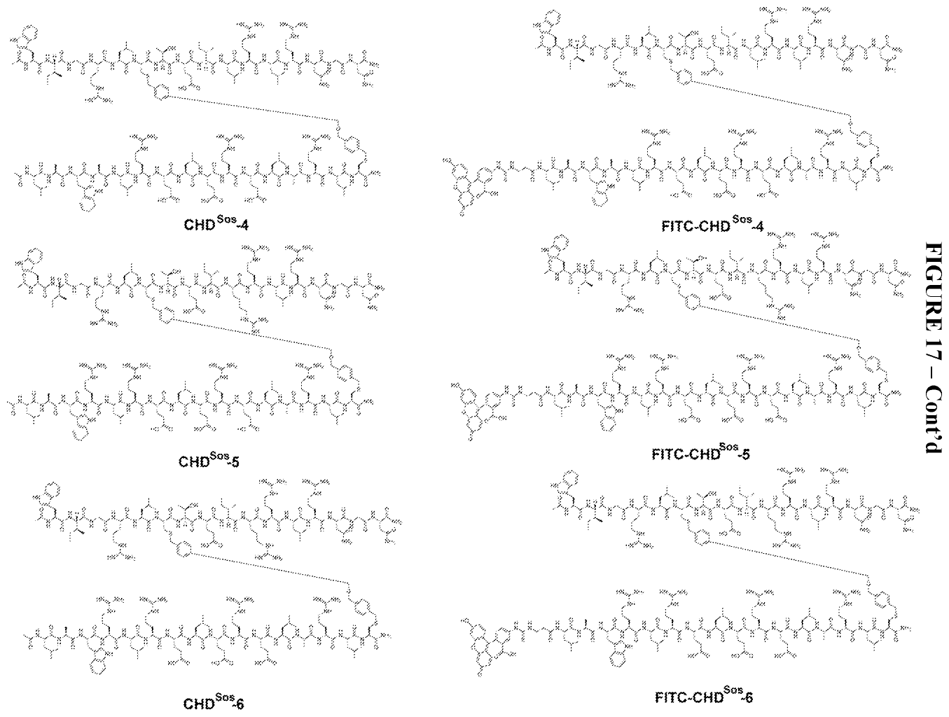

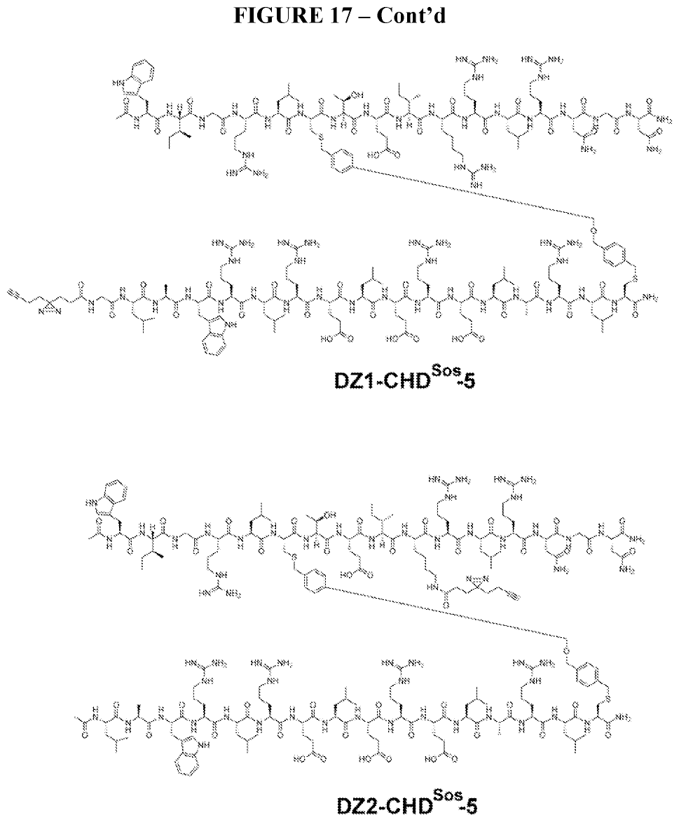

The HPLC traces of Sos CHIDs are provided in FIG. 16. The chemical structures of Sos CHIDs are provided in FIG. 17. The Mass spectroscopic characterization of

CHID peptides is summarized below in Table 1, where R^{H}=L-homoarginine; A_{β}=L-β-alanine; DZ=diazirine photocrosslinker; FITC is 5-fluorescein isothiocyanate linked via thiourea bond to N-terminal amine.

TABLE 1
Mass spectroscopic characterization of CHD peptides ..

| | Sos | | Calculated | Observed |
|---|---|---|---|---|
| Compound | Domain* | Sequence | [M+H]⁺ | [M+H]⁺ |
| CHD^{Sos}-1 | Sos αH | Ac-FIGRLCTEILKLREGN-NH₂ (SEQ | 4180.8 | 4180.8 |
| | | ID NO: 1) | | |
| | Sos αI | Ac-LAWRLRELERELARLC-NH₂ (SEQ | | |
| | | ID NO: 2) | | |
| CHD^{Sos}-2 | Sos αH | Ac-WIGRLCTEILRLRNGN-NH₂ (SEQ | 4248.1 | 4247.0 |
| | | ID NO: 3) | | |
| | Sos αI | Ac-LAWRLRELERELARLC-NH₂ (SEQ | | |
| | | ID NO: 4) | | |
| CHD^{Sos}-3 | Sos αH | Ac-AIGRLCTEILRLRNGA-NH₂ (SEQ | 4089.9 | 4087.9 |
| | | ID NO: 5) | | |
| | Sos αI | Ac-LAWRLRELERELARLC-NH₂ (SEQ | | |
| | | ID NO: 6) | | |
| CHD^{Sos}-4 | Sos αH | Ac-WIGRLCTEILRLRNGN-NH₂ (SEQ | 4162.0 | 4162.0 |
| | | ID NO: 7) | | |
| | Sos αl | Ac-LAWALRELERELARLC-NH₂ (SEQ | | |
| | | ID NO: 8) | | |
| CHD^{Sos}-5 | Sos αH | Ac-WIGRLCTEIRHRLRNGN-NH₂ | 4305.1 | 4304.0 |
| | | (SEQ ID NO: 9) | | |
| | Sos αI | Ac-LAWRLRELERELARLC-NH₂ (SEQ | | |
| | | ID NO: 10) | | |
| CHD^{Sos}-6 | Sos αH | Ac-WIGRLCTEIRRLRNGN-NH₂ (SEQ | 4291.1 | 4289.5 |
| | | ID NO: 11) | | |
| | Sos αI | Ac-LAWRLRELERELARLC-NH₂ (SEQ | | |
| | | ID NO: 12) | | |
| CHD^{Sos}-2 QH | Sos αH | Ac-WIGRLCTEILRLRNGN-NH₂ (SEQ | 1957.3 | 1957.4 |
| | | ID NO: 13) | | |
| CHD^{Sos}-2 aI | Sos αI | Ac-LAWRLRELERELARLC-NH₂ (SEQ | 2069.5 | 2069.4 |
| | | ID NO: 14) | | |
| FITC-CHD^{Sos}-1 | Sos αH | Ac-FIGRLCTEILKLREGN-NH₂ (SEQ | 4599.2 | 4599.0 |
| | | ID NO: 15) | | |
| | Sos αI | FITC-ABLAWRLRELERELARLC-NH₂ | | |
| | | (SEQ ID NO: 16) | | |
| FITC-CHD^{Sos}-2 | Sos αH | Ac-WIGRLCTEILRLRNGN-NH₂ (SEQ | 4666.5 | 4665.5 |
| | | ID NO: 17) | | |
| | Sos αI | FITC-ABLAWRLRELERELARLC-NH₂ | | |
| | | (SEQ ID NO: 18) | | |
| FITC-CHD^{Sos}-3 | Sos αH | Ac-AIGRLCTEILRLRNGA-NH₂ (SEQ | 4508.3 | 4508.0 |
| | | ID NO: 19) | | |
| | Sos αI | FITC-ABLAWRLRELERELARLC-NH₂ | | |
| | | (SEQ ID NO: 20) | | |
| FITC-CHD^{Sos}-4 | Sos αH | Ac-WIGRLCTEILRLRNGN-NH₂ (SEQ | 4580.4 | 4580.1 |
| | | ID NO: 21) | | |
| | Sos αI | FITC-ABLAWALRELERELARLC-NH₂ | | |
| | | (SEQ ID NO: 22) | | |
| FITC-CHD^{Sos}-5 | Sos αH | Ac-WIGRLCTEIRHRLRNGN-NH₂ | 4723.6 | 4722.3 |
| | | (SEQ ID NO: 23) | | |
| | Sos αI | FITC-ABLAWRLRELERELARLC-NH₂ | | |
| | | (SEQ ID NO: 24) | | |
| FITC-CHD^{Sos}-6 | Sos αH | Ac-WIGRLCTEIRRLRNGN-NH₂ (SEQ | 4710.6 | 4710.9 |
| | | ID NO: 25) | | |
| | Sos αI | FITC-ABLAWRLRELERELARLC-NH₂ | | |
| | | (SEQ ID NO: 26) | | |
| DZ1-CHD^{Sos}-5 | Sos αH | Ac-WIGRLCTEIRHRLRNGN-NH₂ | 1117.5 | 1117.6 |
| | | (SEQ ID NO: 27) | [M+4H]4⁺ | |
| | Sos αI | DZ-GLAWRLRELERELARLC-NH₂ | | |
| | | (SEQ ID NO: 28) | | |
| DZ2-CHD^{Sos}-5 | Sos αH | Ac-WIGRLCTEIK(DZ)RLRNGN-NH₂ | 1103.3 | 1102.3 |
| | | (SEQ ID NO: 29) | [M+4H]4⁺ | |
| | Sos αI | Ac-LAWRLRELERELARLC-NH₂ (SEQ | | |
| | | ID NO: 30) | | |

*Mimic of Sos αH or αI sequence with designed changes

### Example 2: Synthesis of Fluorescein-Labeled Peptides

The listed peptide sequences of Table 1, above, were synthesized as previously described with B-alanine added to the N-terminus to serve as a linker prior to coupling with fluorescein isothiocyanate (3 eq.) and DIEA (3 eq.) overnight and protected from light. The fluorophore-conjugated peptides were then cleaved from resin, purified via RP-HPLC, and characterized with MALDI-TOF spectrometry.

### Example 3: Synthesis of Diazirine Photocrosslinker Peptides

The listed peptide sequences (see FIG. 17 and Table 1) were synthesized as previously described with L-glycine added to the N-terminus as a linker prior to coupling of 3-(3-(but-3-ynyl)-3H-diazirin-3-yl)propanoic acid (Sigma-Aldrich) under standard peptide coupling conditions overnight and protected from light. The diazirine-conjugated peptides were then cleaved from resin, purified via RP-HPLC, and characterized with analytical LCMS.

### Example 4: Circular Dichroism

Circular Dichroism (CD) experiments were conducted on an Jasco J-1500 Circular Dichroism spectrometer equipped with a temperature controller using 1 mm length cells and a scan speed of 4.0 nm/min at 298 K. The generated spectra were averaged over 5 scans with baseline subtraction. Raw values were normalized to molar residue ellipticity (MRE). The samples were prepared in a buffer containing 50 mM potassium fluoride in water (pH 7.4) to a final peptide concentration of 20 μM, unless otherwise mentioned. The concentration of each sample was monitored via the UV absorbance at 280 nm of a tryptophan residue.

### Example 5: Protein Purification

Wild-type and mutant N-terminal His6-H-Ras (residues 1-166) in pProEx HTb expression vector were expressed in Escherichia coli (BL21). Cells were grown at 37 ° C. to an absorbance of 0.7 at 600 nm. Protein expression was induced with 500 μM isopropyl β-D-1-thiogalactopyranoside (IPTG) for 6 hours at 30° C. or overnight at 18° C. The bacterial cells were pelleted via centrifugation at 4500 rpm for 15 minutes. Bacterial pellets were resuspended in a lysis buffer containing 20 mM Tris pH 7.4, 300 mM NaCl, 2 mM 2-mercaptoethanol, and a complete, EDTA-free protease inhibitor cocktail (Roche). The lysis, elution and dialysis buffers were also supplemented with 2.5 mM MgCl₂. The resuspended pellets were sonicated using a Branson Cell Disrupter 200. Clarified lysates were formed upon centrifugation at 13,000 rpm for 20 minutes at 4° C. and incubated with charged Ni-NTA resin (Invitrogen) at 4° C. for 1 hour. The resin beads were washed five times with resuspension buffer containing 5 mM imidazole. The His6-tagged proteins were eluted via gravity flow with buffer containing 200 mM imidazole in buffer containing 20 mM Tris, 300 mM NaCl, pH 7.4. Eluted proteins were dialyzed twice against buffer containing 20 mM Tris, 300 mM NaCl, 2.5 mM MgCl₂, pH 7.4 for H-Ras. For HSQC and photoaffinity labeling studies, the polyhistidine tag was cleaved using Tobacco Etch Virus (TEV) protease before proceeding to nucleotide loading. Concentrated eluates were subjected to nucleotide loading and size exclusion chromatography (GE Life Sciences) at 4° C. with 25 mM Tris, 50 mM NaCl, pH 7.5 buffer, and the desired monomeric peaks were collected.

The eluted protein samples were concentrated with 3000 Da molecular weight cutoff Amicon centrifugal columns (Millipore) in dialysis buffer containing 10% glycerol (v/v) before snap-frozen in liquid N₂ and stored at −80° C. until further use.

### Example 6: Nucleotide Loading

Nucleotide loading for fluorescence polarization, HSQC, and nucleotide displacement studies were conducted as previously described in S. M. Margarit et al., Structural evidence for feedback activation by Ras. GTP of the Ras-specific nucleotide exchange factor SOS. Cell 112, 685-695 (2003). Purified H-Ras protein was incubated with GDP or GTP (10 eq.) in loading buffer (20 mM Tris, 300 mM NaCl, 5 mM EDTA, pH 7.4) for 90 minutes on ice. Reactions were then quenched with 12 mM MgCl₂ and incubated for 30 minutes on ice. Depending on the subsequent assay, the protein was subjected to size exclusion chromatography via fast protein liquid chromatography (FPLC) or pre-equilibrated NAP-5 (GE Life Sciences) columns to remove excess free nucleotide and additional purification.

### Example 7: Fluorescence Polarization Binding Assay

The relative binding affinities of CHDs for wild-type and mutant His₆-tagged H-Ras (1-166) pre-loaded with GDP were determined using direct fluorescence polarization assays with fluorescein-tagged CHD peptides (Flu-αHBS, Flu-α₃BHBS). These experiments were conducted with a DTX 880 Multimode Detector (Beckman) at 25° C. with excitation and emission wavelengths set to 485 and 525 nm, respectively. The addition of increasing concentrations of His₆-H-Ras-GDP to a 15 nM solution of fluorescein-labeled CHD in 50 mM Tris, 100 mM NaCl, 0.1% pluronic acid, pH 7.4 was carried out in black, U-bottom 96-well plates (Brand). The generated binding affinity (K_{D}) values for each compound are from biological triplicate studies and were determined via fitting to a sigmoidal dose-response nonlinear regression model on Graphpad Prism 6.

K_{D1}=(R_{T}*(1−F_{SB})+L_{ST}*F_{SB}²)/F_{SB}−L_{ST} (SJ 1)
where:
   R_{T}=Total concentration of H-Ras (1-166)
   L_{ST}=Total concentration of fluorescence peptide
   F_{SB}=Fraction of bound fluorescence peptide

### Example 8: Microscale Thermophoresis (MST)

Fluorescent labeling of the H-Ras protein was performed using the Monolith Protein Labeling Kit RED-MALEIMIDE 2nd Generation from NanoTemper (MO-L014) or AFDye 647 maleimide fluoroprobes (1122-1). Briefly, 20 nM of H-Ras protein was labeled with 1.5 equivalents of cysteine reactive dye in the 1× PBS under dark for 90 minutes. Labeled Ras proteins were then purified via the kit provided column, and the Degree of Label (DOL) was calculated by using the dye's absorbance and protein's original absorbance. The observed DOL value after the labeling for wild-type H-Ras-GDP was 0.43 and G12V H-Ras-GDP was 0.98.

MST binding assays were conducted with NanoTemper Monolith NT.115 Pico. Assay conditions were optimized with a premium coated capillary tube from NanoTemper to avoid random adsorption. Measurement was performed at 25° C. using 10-15% laser excitation power for 20 seconds. The assay buffer was composed of 50 mM Tris, 100 mM NaCl, and 0.1% pluronic acid. 5-10 nM of the dye labeled protein was exposed to serial dilutions of indicated CHDs and incubated for 90 mins in room temperature before the measurements. Error bars are generated from triplicates. Binding affinities were obtained from the MO analysis software in NT.115 Pico with the following equation:

$$f ( Concentration ) = Unbd + \frac{( Bd - Unbd ) \times ( Concentration + TC + K \text{?} - \sqrt{{( Concentration + TC + K \text{?} )}^{2} - 4 \times Concentration \times TC )}}{2 \times TC}$$

$$\text{?} \text{indicates text missing or illegible when filed}$$

where,
   Unbd=Response value of unbound state
   Bd=Response value of bound state
   TC=Final concentration of fluorescent molecule

### Example 9: Serum Stability Assay

Proteolytic stability of CHC^{Sos}-5 was assessed in 25% serum by using Fetal Bovine Serum (FBS, Innovative Research) in RPMI-1640 medium. A 45 μL mixture of 33% FBS (v/v) in serum-free medium was prepared for each sample. The addition of 15 μL of indicated peptide in serum-free medium (25% FBS final) per each experiment was recorded as the starting time point. Time points of 5 min, 6 hours, 12 hours, and 24 hours were analyzed in triplicate. Each experiment was quenched at the determined time point by adding 30 μL 100% EtOH, which was prepared at −70° C. After cooling on ice for 10 minutes, each sample was pelleted at 14,000× g for 5 minutes. 30 μL of the supernatant was isolated, and 3 μL of 500 μM L-Tryptophan was titrated into the sample to be used as an internal standard. The resulting mixture was then immediately subjected to RP-HPLC with a C18 3.5 μm 2.1×150 mm analytical column and monitored at 280 nm. Eluting peaks were collected, and the mass of each peak was determined using MALDI-TOF spectroscopy. Integrated peak area of non-degraded peptides was used to determine the percent surviving in each given condition.

### Example 10: Photoaffinity Labeling and Visualization of the Conjugated CHD-Ras Complex

For each experiment, a sample of 250 μM diazirine-labeled CHD peptide was

incubated with 50 μM of purified H-Ras for 90 minutes at room temperature (100 μL total volume) in binding buffer (50 mM Tris, 100 mM NaCl, 0.1% pluronic acid, pH 7.4). Incubated samples were then photo-irradiated for 5 minutes at 24□C. Photo-conjugated samples were then mixed with 1× SDS-PAGE loading buffer containing 5% 2-mercaptoethanol (200 μL final volume) and heated at 95° C. for 5 minutes. Heated samples were then separated via SDS-PAGE gel electrophoresis on a 1.0 mm 15% polyacrylamide gel. The resulting gel was stained with Coomassie Brilliant Dye and subsequently destained (40% MeOH/10% acetic acid/50% water).

### Example 11: In-solution Tryptic Digest Mass Spectrometry

In-gel tryptic digest was conducted with the In-Gel tryptic Digestion Kit (Thermo Scientific) according the manufacturer's instructions. Upon photoaffinity labeling, the conjugated band was isolated as a 1×1 mm segment for in-gel trypsin digest. The isolated gel band was then incubated with 200 μL of Coomassie destaining solution (25 mM ammonium bicarbonate in 1:1 ultrapure water:acetonitrile) for 30 minutes at 37□C. After incubation, destaining buffer was removed, and the destaining step was repeated twice more. The destained gel band was treated with reducing buffer (25 mM of ammonium bicarbonate, 50 mM TCEP) for 10 minutes at 60° C. After discarding the reducing buffer, the band was treated with 200 μL of iodoacetamide alkylation solution (25 mM ammonium bicarbonate, 100 mM iodoacetamide) for 30 minutes at 37° C. while shaking in the dark. After alkylation, the gel piece was again washed with 100 μL of destaining buffer twice before treatment with 100 μL of acetonitrile for 15 minutes at room temperature. The gel band was then air dried for 15 minutes after removal of the acetonitrile. Trypsin solution was comprised of 5 μg of trypsin protease (Pierce, MS grade) dissolved in 100 μL of 25 mM ammonium bicarbonate buffer, pH 8.0. The dried gel was treated with 100 μL of trypsin solution overnight at 30° C. while shaking. After digestion, the buffer was removed, and the digested peptide was extracted from the gel with 25 μL of 1% trifluoroacetic acid. Extracted samples in 1% trifluoroacetic acid were then diluted with 25 μL of ultrapure water and submitted for MALDI-TOF MS analysis.

### Example 12: Heteronuclear Single Quantum Spectroscopy (HSQC)

The purification procedure is nearly identical to the previously described protocol with a couple exceptions. The E. coli (BL21) cells (4 L) containing the His₆-H-Ras construct were grown at 37° C. in fully supplemented LB broth until O.D. 0.8. The cells were pelleted and resuspended in minimal M9 medium (1 L) before supplemented with 20% glucose and ¹⁵NH₄Cl as the sole nitrogen source (Y. Ito et al., Regional polysterism in the GTP-bound form of the human c-Ha-Ras protein. Biochemistry 36, 9109-9119 (1997)). Protein expression was induced with 1 mM IPTG at O.D. 0.8 overnight at 18° C. Protein purification and subsequent concentration were performed as described above. The His₆-tag was cleaved off upon incubation with recombinant His₆-tagged Tobacco Etch Virus (TEV) protease (Invitrogen) overnight at 4° C. according to the manufacturer's protocol. The resulting protein mixture was loaded onto a charged Ni-NTA column (Invitrogen), and the tag-less protein was collected in the flow-through fraction. ¹⁵N-labeled H-Ras was then pre-loaded with GDP according to the previously mentioned protocol. Uniformly ¹⁵N-labeled H-Ras-GDP underwent buffer exchange into NMR buffer (20 mM Na₂HPO₄—NaH₂PO₄, pH 5.5, 150 mM NaCl, 10 mM MgCl₂) concentrated with an Amicon Ultra centrifugal filter (Millipore), and supplemented with 10% D20 prior to analysis. Data collection was conducted on a 600 MHz Bruker 4-channel NMR system equipped with a 5 mm TCI cryogenic probe at 25° C. using a standard pulse sequence and analyzed by TopSpin 4.0.6 (Bruker). ¹⁵N-¹H-HSQC spectra of 15N-labeled H-Ras-GDP (100 μM) alone was collected, and peaks were assigned based on published data. For peptide titration experiments, 2.5 and 5 equivalents of CHC^{Sos}-5 were dissolved in matching buffer and incubated with ¹⁵N-¹H-Ras (100 μM). The mean chemical shift difference (ΔδNH) observed for ¹H and ¹⁵N nuclei of various resonances were calculated according to the following equation with α=0.14:

ΔδNH={square root over (0.5×[δ_{H}²+(α×δ_{N}²)})

### EXAMPLE 13: Cell Culture

T24, HCT-116, and HeLa cells were maintained in Dulbecco's modified eagle medium (DMEM, Corning Cellgro) supplemented with 10% fetal bovine serum (FBS, Innovative Research), 1× penicillin/streptomycin (EMD Millipore), and 10 mM HEPES buffer (Sigma-Aldrich). H358, Panc 10.05, A549, and BxPC3 cells were maintained in Roswell Park Memorial Institute (RPMI) 1640 medium supplemented with 10% FBS (Innovative Research), 1× penicillin/streptomycin, 10 mM HEPES buffer, and 1× sodium pyruvate (Sigma-Aldrich). All cells were kept in a humidified incubator at 37° C. and 5% CO₂.

### Example 14: Live Cell Fluorescence Microscopy

The indicated cell lines were seeded at 5×10⁵ cells/well in poly-D-lysine coated 35 mm plates (MatTek) and incubated overnight. The growth medium is aspirated and washed with serum-free medium. The cells are then incubated with 1 μM (final) of fluorescein-conjugated peptides dissolved in serum-free medium (0.4% DMSO v/v) for 4 hours at 37° C. while protected from light. All compounds are dissolved as concentrated stocks in DMSO. After the specified incubation time, each plate was aspirated and treated with Hoechst dye solution (Hoechst 33342, ThermoFisher Scientific) for 10 minutes to stain cell nuclei. The Hoechst dye stock solution (10 mg/mL) was diluted 1:2000 in PBS to form the working mixture. The dye solution was removed, and the plate was gently washed 3× with PBS. DMEM (high glucose,

HEPES, no phenol red, 20% FBS) was added to each plate and used as the imaging solution. Fluorescence images were acquired on an Eclipse Ti Fluorescence Microscope (Nikon) equipped with NIS-Elements imaging software and using a 40×objective lens.

### Example 15: Flow Cytometry

The indicated cell lines were seeded at 1×10⁵ cells/well in clear polystyrene 24-well plates (Corning) and incubated overnight. The initial growth medium is replaced with serum-free medium and incubated for 2 hours at 37° C. Upon aspiration, the cells are incubated in serum-free medium containing 0.4% DMSO (v/v) for one hour. The cells were then treated with 1 μM (final) of fluorescein-conjugated peptides in serum-free medium for another hour while protected from light. All compounds are dissolved as concentrated stocks in DMSO. Each well was aspirated and treated with 1× trypsin (0.25% trypsin, 2.21 mM EDTA, Corning Cellgro) for 10 minutes at 37° C. After trypsinization, the resulting solution was mixed with cold serum-free medium and collected. The samples are centrifuged at 500 rpm for 5 minutes at 4ºC.

The supernatant was removed, and the cell pellets were resuspended with cold PBS before placed on ice. Each sample was treated with 10% trypan blue (v/v) immediately before analysis by flow cytometry on a Becton Dickinson Accuri flow cytometer. The presented data consists of the median fluorescence intensities for at least 10,000 cells/sample and processed using FlowJo (Tree Star Inc.).

### Example 16: Cell Viability Assay

Cell viability was monitored using the MTT (Sigma-Aldrich, M2128) or CellTiter-Glo Luminescent cell viability assay (Promega).

### Example 16A: MTT Cell Viability Assay

Cell viability was monitored using the MTT (Sigma-Aldrich, M2128) Luminescent cell viability assay. Cells were plated in clear 96-well plates at 2000 cells/well and allowed to affix overnight at 37° C. Each well is gently aspirated and washed with serum-free medium before introducing peptide inhibitors dissolved in complete medium with 0.5% DMSO (v/v final) to desired concentrations (90 μL/well). The cells are incubated in the presence of peptide for 72 hours at 37° C. MTT reagent solution is composed of Thiazolyl Blue Tetrazolium Bromide dissolved in Dulbecco's Phosphate Buffered Saline (DPBS), pH 7.4 to a concentration of 5 mg/mL and subsequently sterile-filtered into a light-protected container. MTT reagent was added to each well (0.45 mg/mL/well final). The cells are incubated at 37° C. for an additional 4 hours. Upon careful removal of the supernatant, 150 μL DMSO (solubilization solution) is added to each well and mixed to ensure complete solubilization and release of the insoluble purple formazan precipitate into solution. Absorbance values are recorded with a Synergy HT Multi-Detection Microplate Reader (BioTek) at 570 nm.

### Example 16B: CellTiter-Glo Luminescent Cell Viability Assay

Cell viability was monitored using the CellTiter-Glo Luminescent cell viability assay (Promega). Cells were plated in white opaque 96-well plates at 2000 cells/well and allowed to affix overnight at 37° C. Each well is gently aspirated and washed with serum-free medium before introducing peptide inhibitors dissolved in complete medium (DMSO is not needed to dissolve CHC^{Sos}-5) to desired concentrations (90 μ/well). The cells are incubated in the presence of peptide for 72 hours at 37° C. The plate and its contents are then equilibrated to room temperature for approximately 30 minutes after peptide treatment. Upon gentle removal of the well supernatant, 50 μL of 1× X PBS is added to each well. An equivalent volume (50 μL) of CellTiter-Glo 2.0 reagent is then added to each well with the resulting contents mixed for 2 minutes on an orbital shaker to induce cell lysis. The plate is allowed to incubate at room temperature for 10 minutes before recording the luminescent signal (0.25-1 second integration time) with a DTX 880 Multimode Detector (Beckman). Each plate included triplicate wells of a positive death control (cells with 10% DMSO), a negative control (cells with 0.05% DMSO), and a blank (no cells with 0.05% DMSO).

### Example 17: Ras Activation Assay.

H358 cells were initially seeded in 6-well plates (1x106 cells/well) and allowed to attach overnight. The cells were treated with indicated peptides dissolved in complete medium at indicated concentrations for 6 hours at 37° C. Ras activity was determined by the Active Ras Pull-Down and Detection Kit (Thermo Scientific, Catalog number 16117) according to the manufacturer's instructions. In brief, cells were lysed with 250 μL of lysis buffer and scraped off; the resulting lysate was centrifuged at 13,000 rpm for 10 minutes at 4° C. Pre-cleared lysates were subsequently added to 80 μg of GST-tagged RBD and prewashed glutathione agarose beads for 1 hour at 4° C. under constant rocking. The beads were then pelleted, washed 3 times with buffer, and eluted for Western blotting with 50 μL of 2× reducing sample buffer.

### Example 18: ERK Activation Assay.

H358 or HeLa cells were initially seeded in 6-well plates (1x106 cells/well) and allowed to attach overnight. The cells were then incubated with indicated peptides dissolved in complete medium at specified concentrations for 6 hours at 37° C. The cells were washed twice with ice-cold PBS and then lysed in cold RIPA buffer (200 μL/well) containing 25 mM Tris-HCl, pH 7.6, 150 mM NaCl, 1% NP-40, 1% sodium doexycholic acid, 0.1% SDS, Roche Complete Protease inhibitor cocktail (1X, Sigma-Aldrich, P2714), and Roche PhosSTOP phosphatase inhibitor cocktail tablets (1X, Sigma-Aldrich, 4906845001). The cells are kept on ice for 5 minutes with occasional swirling for uniform spreading. Cell lysates are generated with a cell scraper, transferred to a microcentrifuge tube, and centrifuged at 13,000 rpm for 10 minutes at 4° C. The clarified supernatant was collected, and the total protein concentration was measured by Pierce BCA protein assay. The lysates were subjected to SDS-PAGE (10 μg/lane loading) and Western Blot analysis via immunoblotting. Primary antibodies include Phospho-p44/42 MAPK (Erk1/2) (Thr202/Tyr204) (Cell Signaling 4370), p44/42 MAPK (Erk1/2) (Cell Signaling 4695), Ras (Abcam ab 108602), and a-tubulin (Cell Signaling 2144). Secondary antibodies include anti-rabbit IgG, HRP-linked antibody (Cell Signaling 7074P2, 1:2000). After primary antibody incubation, blotted membranes were probed with secondary antibodies and SignalFire ECL Reagent (Cell Signaling 6883P3) and imaged using a ChemiDoc Imaging

System (Bio-Rad). Comparative blot densitometry was performed with ImageJ (NIH) and normalized to tubulin expression.

### Example 19: Preparation and Enrichment of Sample Lysates for Proteomics

H358 cells were grown to 80-95% confluence in 10 cm plates with FBS-supplemented RPMI-1640. The growth medium was aspirated and the cells were washed with Dulbecco's phosphate-buffered saline (DPBS). The cells were then incubated with serum-free medium containing probes DZ2-CHC^{Sos}-5 (20 μM in RPMI-1640 with 0.4% DMSO, two replicates) or control probe CP-2-66 (20 μM in RPMI-1640 with 0.4% DMSO, four replicates) for 4 hours at 37° C. under an atmosphere of 5% CO₂ before being irradiated under UV light (365 nm, 30 min, 4° C.). The cells were washed with DPBS, scraped and transferred to 1.5 mL Eppendorf tubes, pelleted, and stored at −80° C. until the next step.

Cell pellets were resuspended in 500 μL of DPBS and lysed by sonication (15 ms on, 40 ms off, 15% amplitude, 1 s total on×2). Protein concentrations were normalized (1-2 mg/mL; final volume of 500 μL with DPBS) using the Lowry Protein Assay (Pierce). To each sample was added solutions of tris((1-benzyl-4-triazolyl)methyl)amine (30 μl, 1.7 mM in DMSO/t-BuOH 1:4 v/v), tris(2-carboxyethyl)phosphine (10 μL, 50 mM), biotin-PEG3-azide (5 μL, 100 μM), and CuSO4 (10 μL, 50 mM) and the samples were shaken at room temperature for 1 hour. A cold MeOH/CHCI3 mixture (2.5 mL, 4:1 v/v) was added to each lysate, followed by 1 mL of cold DPBS. The resulting mixture was vortexed and centrifuged (4,700× g, 10 min, 4° C.). The organic and aqueous layers were aspirated, and the remaining protein disk was further washed via sonication in cold MeOH/CHCl₃ solution (2 mL, 4:1) and pelleted by centrifugation (4,700× g, 10 min, 4° C.). The protein pellet was aspirated and combined with freshly prepared urea solution (500 μL, 6 M in DPBS) and a solution of SDS (with 10 μL of 10% w/v) by sonication. A freshly prepared 1:1 solution (50 μL) of TCEP (200 mM in DPBS) and K₂CO₃ (600 mM in DPBS) was added and the mixture was incubated for 30 min at 37° C. with shaking. A solution of freshly prepared iodoacetamide (70 μL, 400 mM in DPBS) was added and the mixture was incubated at RT in the absence of light. Each sample was combined with a solution of SDS (130 μL, 10% in DPBS w/v) followed by DPBS (5.5 mL) and incubated with pre-equilibrated streptavidin-agarose beads (100 μL of a 50% slurry; Pierce) for 1.5 hours at RT while rotating. The streptavidin beads were pelleted by centrifugation (750 g, 2 min, 4° C.) and sequentially washed with SDS solution (1×5 mL, 0.2% in DPBS), DPBS (2×5 mL), and triethylammonium bicarbonate buffer (1×5 mL, TEAB, 100 mM, pH 8.4). The beads were resuspended in TEAB (0.5 mL, 100 mM pH 8.5) and transferred into LoBind microcentrifuge tubes. The tube was washed once more with TEAB (0.5 mL, 100 mM pH 8.5) to ensure complete transfer of the beads, which were then pelleted by centrifugation (750× g, 2 min, 4° C. and the supernatant aspirated. Each sample of beads was combined with a solution of CaCl₂ (2 μL, 100 mM) and a solution of sequencing-grade porcine trypsin (2 μg, Promega in 200 μL TEAB, 100 mM, pH 8.4), and incubated for 14 hours at 37° C.with shaking. The beads were pelleted by centrifugation (750× g, 2 min, 4° C.) and the supernatants were transferred to new

LoBind micro-centrifuge tubes. Each digested sample was TMT 10plex labeled (Thermo Fisher Scientific): for each sample a stock solution of TMT reagent (8 μL, 20 μg/μL) was added along with dry MS-grade acetonitrile (final acetonitrile concentration 30% v/v), followed by incubation at RT for 1 hour. The reaction was quenched by adding hydroxylamine (6 μL) and left to stand for 15 min, followed by the addition of formic acid (5 μL). Each TMT-labeled sample was dried via vacuum centrifugation, and the samples were combined by redissolving one sample in a solution of trifluoroacetic acid (TFA, 200 μL, 0.1% in water) and transferring the solution into each sample tube until all samples were redissolved. The process was repeated with a further volume of TFA solution (100 L, in water, final volume 300 μL) and the combined sample was dried via vacuum centrifugation. The samples were fractionated using a fractionation kit (Pierce

High pH Reversed-Phase Peptide Fractionation Kit) according to the manufacturer's instructions. Briefly, the peptide fractions were eluted from reversed-phase spin columns with consecutive solutions of 0.1% triethylamine combined with MeCN (5%, 7.5%, 10%, 12.5%, 15%, 17.5%, 20%, 22.5%, 25%, 30%, 50%, 95% MeCN). The fractions were combined pairwise (fraction 1 with fraction 7, fraction 2 with fraction 8, etc.), dried by vacuum centrifugation, and stored at −80° C. until ready for injection.

### Example 20: LC-MS Analysis for Proteomics

TMT samples were analyzed using a Thermo Fisher Scientific Orbitrap Fusion Lumos mass spectrometer equipped with an UltiMate 3000 RSLCnano system (Thermo Fisher Scientific) following previously reported procedures (Y. Wang et al., Expedited mapping of the ligandable proteome using fully functionalized enantiomeric probe pairs. Nat. Chem. 11, 1113-1123 (2019); E. Joeh et al., Mapping glycan-mediated galectin-3 interactions by live cell proximity labeling. Proc. Natl. Acad. Sci. USA 117, 27329-27338 (2020)).

Dissolved samples (20 μl; 0.1%, v v formic acid in water) were injected (3 μl/injection) on to an Acclaim PepMap RSLC analytical column (75 μm×15 cm) equipped with an Acclaim PepMap 100 precolumn (75 μm×2 mm) and eluted using the following gradient (300 μl/min, column temperature 35° C.): 2% buffer B (0.1% formic acid in acetonitrile) and 98% buffer A (0.1% formic acid in water) for 10 min; buffer B increased to 30% over 192 min, then to 60% over 6 min, followed by an increase to 95% over 1 min and held steady for 5 min;

buffer B was decreased to 2% over 1 min where it remained for 6 min. The voltage applied to the nano-LC electrospray source was 2.0 kV and MS¹ spectra were acquired at a resolution of 120,000 with an automatic gain control (AGC) target value of 1×10⁶ ions and a maximum injection time of 50 ms. A data-dependent acquisition mode was used (repeat count 1, duration 20 s), with a scan range of 375 to 1,500 m/z. Collision-induced dissociation (CID) was performed for MS² peptide fragmentation (quadrupole ion trap analysis, AGC 1.8×10⁴, CID collision energy 30%, maximum injection time 120 ms, isolation window 1.6) and the MS³ precursor was fragmented through high-energy collision-induced dissociation (HCD, collision energy 65%). Synchronous precursor selection (SPS) was enabled to include up to 10 MS² fragment ions for the MS³ spectrum, detected with the Orbitrap (resolution of 50,000, AGC target value of 1.5×10⁵, maximum injection time of 120 ms).

### Example 21: Proteomics Data Analysis

Data processing was performed as in a previously reported procedure (E. Joeh et al., Mapping glycan-mediated galectin-3 interactions by live cell proximity labeling. Proc. Natl. Acad. Sci. USA 117, 27329-27338 (2020)). The Proteome Discoverer 2.4 software package (Thermo Fisher Scientific) was used to determine peptide sequences using the Homo sapiens proteome database (42,358 sequences) via SEQUEST HT algorithm (10 ppm precursor mass tolerance, 0.6 Da fragment ion mass tolerance, one missed cleavage allowed, target false discovery rate 1% (Percolator)). Static modifications were set for arbamidomethyl (C, +57.02146) and the TMT-tag (K and N-terminal, +229.1629), while oxidation (M, +15.994915) as a variable modification. Peptide quantitation was performed on the MS3 level with reporter ion mass tolerance set to 20 ppm. Subsequent statistical analysis was performed in Python 3.6.5. TMT abundances and ratios obtained from Proteome Discoverer were transformed with log₂(x), and p-values were obtained using t-tests with two biological replicates. Additionally, identified proteins were required to have a minimum of three unique peptides. Quantitative data are listed in Table 2, below. The mass spectrometric proteomics datasets have been deposited to the MassIVE repository with the dataset identifier MSV000086972.

TABLE 2
Enriched proteins from MS-based proteomics using photoaffinity crosslinking of DZ2-
CHD^{Sos}-5 in H358 cells.

| | | | | Average | | | | | |
|---|---|---|---|---|---|---|---|---|---|
| | | | | Abundance | | Fold | p-value | log₂(fold) | −log₁₀(p) |
| | # | # Unique | | DZ2- | CP- | DZ2- | DZ2- | DZ2- | DZ2- |
| Accession | Peptides | Peptides | Gene Symbol | CHD^{Sos}-5 | 266ª | CHD^{Sos}-5 | CHD^{Sos}-5 | CHD^{Sos}-5 | CHD^{Sos}-5 |
| P62826 | 11 | 11 | RAN | 7714 | 964 | 8.00 | 2.0E−08 | 3.00 | 7.69 |
| P19623 | 7 | 7 | SRM | 1300 | 143 | 9.12 | 1.2E−07 | 3.19 | 6.92 |
| P21964 | 7 | 7 | COMT | 1550 | 210 | 7.37 | 1.4E−07 | 2.88 | 6.85 |
| P50914 | 6 | 6 | RPL14 | 1804 | 284 | 6.36 | 1.8E−07 | 2.67 | 6.75 |
| Q99497 | 3 | 3 | PARK7 | 1518 | 305 | 4.98 | 1.9E−07 | 2.32 | 6.71 |
| Q02543 | 7 | 7 | RPL18A | 6677 | 1077 | 6.20 | 2.0E−07 | 2.63 | 6.70 |
| P51153 | 5 | 3 | RAB13 | 198 | 27 | 7.35 | 2.0E−07 | 2.88 | 6.70 |
| O75821 | 9 | 9 | EIF3G | 2111 | 276 | 7.64 | 2.2E−07 | 2.93 | 6.65 |
| P62424 | 15 | 15 | RPL7A | 5704 | 886 | 6.44 | 2.4E−07 | 2.69 | 6.62 |
| Q9GZZ1 | 7 | 7 | NAA50 | 1429 | 201 | 7.11 | 2.6E−07 | 2.83 | 6.58 |
| Q9H7E9 | 3 | 3 | C8orf33 | 541 | 64 | 8.46 | 2.7E−07 | 3.08 | 6.57 |
| P39019 | 11 | 11 | RPS19 | 9543 | 855 | 11.17 | 3.0E−07 | 3.48 | 6.53 |
| Q9BYD2 | 6 | 6 | MRPL9 | 895 | 140 | 6.40 | 3.0E−07 | 2.68 | 6.52 |
| Q9Y6C9 | 5 | 5 | MTCH2 | 1152 | 202 | 5.70 | 3.4E−07 | 2.51 | 6.46 |
| Q99816 | 9 | 9 | TSG101 | 1485 | 174 | 8.53 | 4.4E−07 | 3.09 | 6.36 |
| Q12765 | 5 | 5 | SCRN1 | 625 | 75 | 8.29 | 5.0E−07 | 3.05 | 6.30 |
| Q15185 | 8 | 8 | PTGES3 | 2760 | 228 | 12.08 | 5.5E−07 | 3.59 | 6.26 |
| P22087 | 10 | 10 | FBL | 3540 | 697 | 5.08 | 6.1E−07 | 2.34 | 6.21 |
| P68104 | 25 | 13 | EEF1A1 | 33254 | 3786 | 8.78 | 6.8E−07 | 3.13 | 6.17 |
| O75608 | 5 | 5 | LYPLA1 | 1898 | 368 | 5.16 | 7.1E−07 | 2.37 | 6.15 |
| Q9H0S4 | 9 | 9 | DDX47 | 1727 | 254 | 6.81 | 7.9E−07 | 2.77 | 6.10 |
| P61026 | 13 | 10 | RAB10 | 7046 | 1160 | 6.08 | 8.6E−07 | 2.60 | 6.07 |
| P61106 | 14 | 13 | RAB14 | 7072 | 1053 | 6.72 | 9.2E−07 | 2.75 | 6.04 |
| 095470 | 11 | 11 | SGPL1 | 717 | 122 | 5.87 | 9.2E−07 | 2.55 | 6.04 |
| Q5BKZ1 | 3 | 3 | ZNF326 | 89 | 14 | 6.21 | 1.3E−06 | 2.64 | 5.89 |
| Q15691 | 15 | 15 | MAPRE1 | 2720 | 360 | 7.55 | 1.3E−06 | 2.92 | 5.89 |
| Q9BWF3 | 12 | 12 | RBM4 | 2761 | 355 | 7.78 | 1.4E−06 | 2.96 | 5.86 |
| Q08174 | 6 | 6 | PCDH1 | 1258 | 157 | 8.00 | 1.4E−06 | 3.00 | 5.86 |
| Q9BRU9 | 3 | 3 | UTP23 | 631 | 141 | 4.47 | 1.4E−06 | 2.16 | 5.84 |
| P62906 | 9 | 9 | RPL10A | 12200 | 1574 | 7.75 | 1.6E−06 | 2.95 | 5.79 |
| Q15050 | 6 | 6 | RRS1 | 1090 | 222 | 4.91 | 1.9E−06 | 2.30 | 5.71 |
| P06493 | 8 | 8 | CDK1 | 2291 | 269 | 8.52 | 2.1E−06 | 3.09 | 5.68 |
| Q9Y3B7 | 6 | 6 | MRPL11 | 2437 | 312 | 7.81 | 2.4E−06 | 2.97 | 5.62 |
| Q14684 | 3 | 3 | RRP1B | 778 | 167 | 4.67 | 2.4E−06 | 2.22 | 5.61 |
| P23497 | 5 | 4 | SP100 | 453 | 78 | 5.81 | 2.7E−06 | 2.54 | 5.56 |
| P38919 | 22 | 19 | EIF4A3 | 6326 | 1033 | 6.13 | 2.9E−06 | 2.61 | 5.54 |
| Q9NP72 | 6 | 6 | RAB18 | 1628 | 271 | 6.01 | 3.3E−06 | 2.59 | 5.48 |
| Q9BQA1 | 3 | 3 | WDR77 | 67 | 13 | 5.35 | 3.7E−06 | 2.42 | 5.44 |
| P62854 | 3 | 3 | RPS26 | 743 | 106 | 6.98 | 3.8E−06 | 2.80 | 5.42 |
| Q8TDB6 | 3 | 3 | DTX3L | 151 | 47 | 3.19 | 4.2E−06 | 1.67 | 5.38 |
| Q96A08 | 7 | 3 | HIST1H2BA | 1514 | 390 | 3.89 | 5.0E−06 | 1.96 | 5.30 |
| P39023 | 26 | 26 | RPL3 | 13403 | 1468 | 9.13 | 7.7E−06 | 3.19 | 5.11 |
| P47755 | 8 | 5 | CAPZA2 | 521 | 74 | 7.09 | 8.3E−06 | 2.83 | 5.08 |
| P36404 | 5 | 5 | ARL2 | 665 | 75 | 8.87 | 9.0E−06 | 3.15 | 5.05 |
| P52597 | 10 | 8 | HNRNPF | 2729 | 425 | 6.42 | 9.9E−06 | 2.68 | 5.01 |
| P05787 | 45 | 37 | KRT8 | 59794 | 13117 | 4.56 | 9.9E−06 | 2.19 | 5.01 |
| P22392-2 | 13 | 5 | NME2 | 5458 | 1020 | 5.35 | 1.0E−05 | 2.42 | 4.99 |
| P52815 | 4 | 4 | MRPL12 | 156 | 35 | 4.47 | 1.1E−05 | 2.16 | 4.95 |
| P18124 | 17 | 17 | RPL7 | 9362 | 1364 | 6.87 | 1.1E−05 | 2.78 | 4.94 |
| P27707 | 3 | 3 | DCK | 333 | 51 | 6.51 | 1.2E−05 | 2.70 | 4.93 |
| Q9H8Y8 | 4 | 4 | GORASP2 | 202 | 46 | 4.34 | 1.4E−05 | 2.12 | 4.86 |
| Q9Y496 | 6 | 6 | KIF3A | 399 | 73 | 5.48 | 1.5E−05 | 2.46 | 4.82 |
| Q9NTK5 | 9 | 9 | OLA1 | 1492 | 253 | 5.90 | 2.6E−05 | 2.56 | 4.59 |
| Q8WYA6 | 12 | 12 | CTNNBL1 | 2201 | 322 | 6.83 | 2.6E−05 | 2.77 | 4.58 |
| P09493-3 | 16 | 5 | TPM1 | 2215 | 527 | 4.20 | 3.0E−05 | 2.07 | 4.52 |
| P60660 | 6 | 6 | MYL6 | 2017 | 395 | 5.10 | 3.3E−05 | 2.35 | 4.49 |
| Q5JWF2 | 5 | 4 | GNAS | 104 | 19 | 5.62 | 3.3E−05 | 2.49 | 4.48 |
| Q9GZZ9 | 3 | 3 | UBA5 | 413 | 51 | 8.06 | 3.4E−05 | 3.01 | 4.47 |
| P09211 | 8 | 8 | GSTP1 | 4548 | 947 | 4.80 | 5.1E−05 | 2.26 | 4.29 |
| Q96FQ6 | 4 | 4 | S100A16 | 2466 | 426 | 5.79 | 6.1E−05 | 2.53 | 4.21 |
| Q9H7Z7 | 14 | 14 | PTGES2 | 4174 | 521 | 8.01 | 6.6E−05 | 3.00 | 4.18 |
| P17931 | 6 | 6 | LGALS3 | 2583 | 517 | 4.99 | 6.7E−05 | 2.32 | 4.17 |
| O00299 | 8 | 8 | CLIC1 | 3016 | 340 | 8.88 | 6.8E−05 | 3.15 | 4.16 |
| O60506-3 | 28 | 23 | SYNCRIP | 16364 | 2845 | 5.75 | 7.3E−05 | 2.52 | 4.14 |
| Q13243 | 5 | 4 | SRSF5 | 553 | 145 | 3.81 | 8.0E−05 | 1.93 | 4.10 |
| Q8TDD1 | 9 | 9 | DDX54 | 1424 | 405 | 3.52 | 8.9E−05 | 1.81 | 4.05 |
| Q9BTY7 | 4 | 4 | HGH1 | 506 | 93 | 5.45 | 9.5E−05 | 2.45 | 4.02 |
| P27144 | 5 | 5 | AK4 | 1250 | 154 | 8.11 | 1.1E−04 | 3.02 | 3.97 |
| P54819 | 6 | 6 | AK2 | 1082 | 335 | 3.23 | 1.1E−04 | 1.69 | 3.94 |
| P45973 | 6 | 6 | CBX5 | 4248 | 832 | 5.11 | 1.2E−04 | 2.35 | 3.92 |
| Q9UNX4 | 5 | 5 | WDR3 | 235 | 72 | 3.24 | 1.3E−04 | 1.69 | 3.88 |
| Q5T6V5 | 5 | 5 | C9orf64 | 420 | 55 | 7.71 | 1.5E−04 | 2.95 | 3.83 |
| Q07666 | 12 | 12 | KHDRBS1 | 9696 | 1587 | 6.11 | 1.7E−04 | 2.61 | 3.76 |
| Q15785 | 9 | 9 | TOMM34 | 1723 | 156 | 11.08 | 2.4E−04 | 3.47 | 3.61 |
| Q9BVP2 | 12 | 12 | GNL3 | 2113 | 441 | 4.80 | 2.7E−04 | 2.26 | 3.57 |
| Q9B VI4 | 5 | 5 | NOC4L | 366 | 73 | 5.03 | 2.9E−04 | 2.33 | 3.54 |
| Q9BQ67 | 5 | 5 | GRWD1 | 1045 | 311 | 3.36 | 3.0E−04 | 1.75 | 3.52 |
| Q9Y2X3 | 10 | 10 | NOP58 | 2414 | 869 | 2.78 | 3.1E−04 | 1.47 | 3.51 |
| P56182 | 7 | 7 | RRP1 | 680 | 174 | 3.91 | 3.2E−04 | 1.97 | 3.50 |
| O60841 | 10 | 10 | EIF5B | 689 | 158 | 4.37 | 3.2E−04 | 2.13 | 3.49 |
| P54619 | 9 | 9 | PRKAG1 | 3113 | 336 | 9.27 | 3.5E−04 | 3.21 | 3.45 |
| Q8WUA2 | 3 | 3 | PPIL4 | 189 | 33 | 5.66 | 3.5E−04 | 2.50 | 3.45 |
| P01116-2 | 6 | 6 | KRAS | 825 | 124 | 6.63 | 3.7E−04 | 2.73 | 3.43 |
| Q9BRK5 | 6 | 6 | SDF4 | 633 | 90 | 7.02 | 3.9E−04 | 2.81 | 3.41 |
| Q9Y3D6 | 3 | 3 | FIS1 | 357 | 126 | 2.84 | 4.2E−04 | 1.51 | 3.37 |
| P31146 | 5 | 4 | CORO1A | 523 | 117 | 4.47 | 5.6E−04 | 2.16 | 3.25 |
| P62280 | 9 | 9 | RPS11 | 6290 | 579 | 10.86 | 5.6E−04 | 3.44 | 3.25 |
| Q15070 | 4 | 4 | OXAIL | 186 | 37 | 5.08 | 5.6E−04 | 2.34 | 3.25 |
| Q8IY81 | 15 | 15 | FTSJ3 | 2367 | 788 | 3.01 | 5.7E−04 | 1.59 | 3.24 |
| Q08257 | 4 | 4 | CRYZ | 254 | 70 | 3.61 | 5.7E−04 | 1.85 | 3.24 |
| Q14137 | 6 | 6 | BOP1 | 631 | 163 | 3.87 | 5.8E−04 | 1.95 | 3.23 |
| Q9NVP1 | 16 | 16 | DDX18 | 2793 | 821 | 3.40 | 6.2E−04 | 1.77 | 3.21 |
| O96008 | 8 | 8 | TOMM40 | 3359 | 735 | 4.57 | 6.3E−04 | 2.19 | 3.20 |
| Q86SX6 | 3 | 3 | GLRX5 | 357 | 85 | 4.21 | 6.3E−04 | 2.07 | 3.20 |
| P46777 | 11 | 11 | RPL5 | 6090 | 837 | 7.27 | 6.6E−04 | 2.86 | 3.18 |
| P35080-2 | 4 | 3 | PFN2 | 1570 | 232 | 6.76 | 7.1E−04 | 2.76 | 3.15 |
| Q10713 | 4 | 4 | PMPCA | 395 | 100 | 3.94 | 7.3E−04 | 1.98 | 3.14 |
| P62851 | 5 | 5 | RPS25 | 5841 | 577 | 10.13 | 7.8E−04 | 3.34 | 3.11 |
| P06753-2 | 24 | 12 | TPM3 | 16084 | 3226 | 4.99 | 8.4E−04 | 2.32 | 3.08 |
| Q8WU90 | 9 | 9 | ZC3H15 | 270 | 39 | 6.94 | 9.6E−04 | 2.79 | 3.02 |
| Q8NEJ9 | 3 | 3 | NGDN | 251 | 45 | 5.53 | 1.1E−03 | 2.47 | 2.96 |
| Q99614 | 6 | 6 | TTC1 | 1189 | 183 | 6.51 | 1.1E−03 | 2.70 | 2.94 |
| P28066 | 6 | 6 | PSMA5 | 2172 | 340 | 6.39 | 1.2E−03 | 2.68 | 2.94 |
| P30419 | 4 | 4 | NMT1 | 480 | 165 | 2.90 | 1.4E−03 | 1.54 | 2.86 |
| Q13206 | 3 | 3 | DDX10 | 87 | 19 | 4.64 | 1.4E−03 | 2.22 | 2.84 |
| P05783 | 31 | 30 | KRT18 | 34924 | 9662 | 3.61 | 1.5E−03 | 1.85 | 2.83 |
| P31943 | 14 | 8 | HNRNPH1 | 11191 | 1588 | 7.05 | 1.5E−03 | 2.82 | 2.82 |
| O75844 | 4 | 4 | ZMPSTE24 | 1308 | 482 | 2.71 | 1.6E−03 | 1.44 | 2.80 |
| P27635 | 10 | 10 | RPL10 | 8043 | 984 | 8.18 | 1.7E−03 | 3.03 | 2.78 |
| P62258 | 22 | 20 | YWHAE | 16731 | 1848 | 9.05 | 1.7E−03 | 3.18 | 2.76 |
| Q9BUQ8 | 5 | 5 | DDX23 | 207 | 68 | 3.05 | 1.8E−03 | 1.61 | 2.75 |
| P60842 | 25 | 13 | EIF4A1 | 13939 | 1642 | 8.49 | 1.8E−03 | 3.09 | 2.74 |
| Q9H074 | 3 | 3 | PAIP1 | 276 | 45 | 6.18 | 1.8E−03 | 2.63 | 2.74 |
| P61981 | 10 | 6 | YWHAG | 2231 | 261 | 8.53 | 2.0E−03 | 3.09 | 2.70 |
| P63241 | 10 | 10 | EIF5A | 5906 | 792 | 7.46 | 2.0E−03 | 2.90 | 2.69 |
| Q13247 | 6 | 5 | SRSF6 | 2200 | 666 | 3.30 | 2.0E−03 | 1.72 | 2.69 |
| Q9H3Q1 | 4 | 4 | CDC42EP4 | 535 | 48 | 11.13 | 2.1E−03 | 3.48 | 2.68 |
| P27348 | 14 | 9 | YWHAQ | 5706 | 648 | 8.81 | 2.1E−03 | 3.14 | 2.68 |
| P82930 | 3 | 3 | MRPS34 | 717 | 101 | 7.08 | 2.2E−03 | 2.82 | 2.67 |
| Q14498 | 6 | 6 | RBM39 | 1230 | 425 | 2.90 | 2.2E−03 | 1.53 | 2.66 |
| O76021 | 20 | 20 | RSL1D1 | 5114 | 1655 | 3.09 | 2.3E−03 | 1.63 | 2.64 |
| O95758-1 | 8 | 5 | PTBP3 | 333 | 55 | 6.06 | 2.5E−03 | 2.60 | 2.61 |
| Q6P1J9 | 3 | 3 | CDC73 | 251 | 58 | 4.34 | 2.5E−03 | 2.12 | 2.60 |
| Q53GQ0 | 10 | 10 | HSD17B12 | 3717 | 495 | 7.51 | 2.6E−03 | 2.91 | 2.59 |
| Q9HAV4 | 4 | 4 | XPO5 | 197 | 49 | 3.98 | 2.8E−03 | 1.99 | 2.55 |
| Q96S44 | 4 | 4 | TP53RK | 596 | 52 | 11.54 | 3.0E−03 | 3.53 | 2.53 |
| PODP25 | 5 | 5 | CALM3 | 1088 | 211 | 5.16 | 3.2E−03 | 2.37 | 2.50 |
| Q9ULX3 | 3 | 3 | NOB1 | 233 | 23 | 9.99 | 3.2E−03 | 3.32 | 2.49 |
| P19338 | 42 | 42 | NCL | 21643 | 8238 | 2.63 | 3.3E−03 | 1.39 | 2.49 |
| P78318 | 6 | 6 | IGBP1 | 660 | 74 | 8.91 | 3.3E−03 | 3.16 | 2.48 |
| Q05639 | 22 | 10 | EEF1A2 | 4035 | 438 | 9.22 | 3.3E−03 | 3.20 | 2.48 |
| P24534 | 9 | 7 | EEF1B2 | 6809 | 646 | 10.55 | 3.4E−03 | 3.40 | 2.47 |
| P19784 | 7 | 7 | CSNK2A2 | 812 | 112 | 7.25 | 3.5E−03 | 2.86 | 2.46 |
| Q9ULC4 | 4 | 4 | MCTS1 | 484 | 82 | 5.88 | 3.5E−03 | 2.56 | 2.45 |
| Q96A49 | 11 | 11 | SYAP1 | 966 | 109 | 8.88 | 3.7E−03 | 3.15 | 2.43 |
| Q99439 | 6 | 4 | CNN2 | 791 | 186 | 4.24 | 3.8E−03 | 2.09 | 2.42 |
| Q15628 | 4 | 4 | TRADD | 74 | 17 | 4.34 | 4.0E−03 | 2.12 | 2.39 |
| O60547 | 5 | 5 | GMDS | 261 | 32 | 8.12 | 4.1E−03 | 3.02 | 2.38 |
| P04632 | 6 | 6 | CAPNS1 | 555 | 87 | 6.35 | 4.2E−03 | 2.67 | 2.37 |
| P40939 | 22 | 22 | HADHA | 4549 | 1190 | 3.82 | 4.5E−03 | 1.94 | 2.35 |
| Q9BYG3 | 5 | 5 | NIFK | 1813 | 344 | 5.27 | 4.8E−03 | 2.40 | 2.32 |
| Q01081 | 4 | 4 | U2AF1 | 396 | 85 | 4.66 | 4.8E−03 | 2.22 | 2.32 |
| P49720 | 3 | 3 | PSMB3 | 422 | 159 | 2.66 | 5.2E−03 | 1.41 | 2.28 |
| Q9BUF5 | 18 | 8 | TUBB6 | 2620 | 284 | 9.22 | 5.2E−03 | 3.21 | 2.28 |
| P61224 | 3 | 3 | RAP1B | 1520 | 227 | 6.69 | 5.6E−03 | 2.74 | 2.25 |
| P35659 | 11 | 11 | DEK | 2440 | 785 | 3.11 | 5.7E−03 | 1.64 | 2.25 |
| P20042 | 12 | 12 | EIF2S2 | 5270 | 893 | 5.90 | 5.8E−03 | 2.56 | 2.24 |
| Q99747 | 6 | 6 | NAPG | 98 | 18 | 5.47 | 6.2E−03 | 2.45 | 2.20 |
| P35222 | 17 | 15 | CTNNB1 | 4435 | 1217 | 3.64 | 6.4E−03 | 1.87 | 2.20 |
| Q96FW1 | 7 | 7 | OTUB1 | 3921 | 446 | 8.79 | 6.5E−03 | 3.14 | 2.18 |
| Q01105 | 12 | 3 | SET | 5660 | 1233 | 4.59 | 6.6E−03 | 2.20 | 2.18 |
| P55795 | 10 | 5 | HNRNPH2 | 1682 | 230 | 7.30 | 6.6E−03 | 2.87 | 2.18 |
| P51148 | 8 | 6 | RAB5C | 2640 | 450 | 5.87 | 6.7E−03 | 2.55 | 2.17 |
| P61081 | 6 | 6 | UBE2M | 1401 | 229 | 6.13 | 6.7E−03 | 2.62 | 2.17 |
| Q9P2K5 | 4 | 4 | MYEF2 | 262 | 44 | 5.98 | 6.8E−03 | 2.58 | 2.17 |
| Q99829 | 3 | 3 | CPNE1 | 215 | 35 | 6.17 | 6.9E−03 | 2.62 | 2.16 |
| Q9P287 | 4 | 4 | BCCIP | 693 | 94 | 7.38 | 6.9E−03 | 2.88 | 2.16 |
| Q92733 | 5 | 5 | PRCC | 893 | 160 | 5.59 | 7.0E−03 | 2.48 | 2.15 |
| Q9BX10 | 3 | 3 | GTPBP2 | 91 | 11 | 8.15 | 7.1E−03 | 3.03 | 2.15 |
| P50402 | 10 | 10 | EMD | 3191 | 480 | 6.64 | 7.2E−03 | 2.73 | 2.14 |
| O14773 | 3 | 3 | TPP1 | 113 | 32 | 3.49 | 7.4E−03 | 1.80 | 2.13 |
| Q99627 | 4 | 4 | COPS8 | 1199 | 190 | 6.31 | 7.6E−03 | 2.66 | 2.12 |
| P39687 | 9 | 4 | ANP32A | 2059 | 284 | 7.26 | 7.9E−03 | 2.86 | 2.10 |
| P09651 | 18 | 13 | HNRNPA1 | 10688 | 2198 | 4.86 | 8.0E−03 | 2.28 | 2.10 |
| P30484 | 7 | 4 | HLA-B | 415 | 86 | 4.85 | 8.3E−03 | 2.28 | 2.08 |
| P63104 | 18 | 13 | YWHAZ | 16707 | 1983 | 8.42 | 8.4E−03 | 3.07 | 2.07 |
| O43390 | 27 | 22 | HNRNPR | 10264 | 1745 | 5.88 | 8.6E−03 | 2.56 | 2.07 |
| P29692 | 10 | 8 | EEF1D | 6725 | 614 | 10.95 | 9.0E−03 | 3.45 | 2.04 |
| Q9P289 | 9 | 5 | STK26 | 1337 | 170 | 7.87 | 9.3E−03 | 2.98 | 2.03 |
| P60510 | 3 | 3 | PPP4C | 377 | 60 | 6.27 | 9.8E−03 | 2.65 | 2.01 |
| Q15126 | 8 | 8 | PMVK | 1594 | 177 | 9.02 | 9.9E−03 | 3.17 | 2.00 |
| O95433 | 12 | 12 | AHSA1 | 1074 | 157 | 6.84 | 9.9E−03 | 2.77 | 2.00 |

ªCP-266 = 3-(3-(but-3-yn-1-yl)-3H-diazirin-3-yl)-N-methylpropanamide structure is

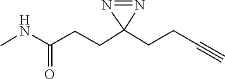

### Example 22: Design of Sos Tertiary Helix Mimics

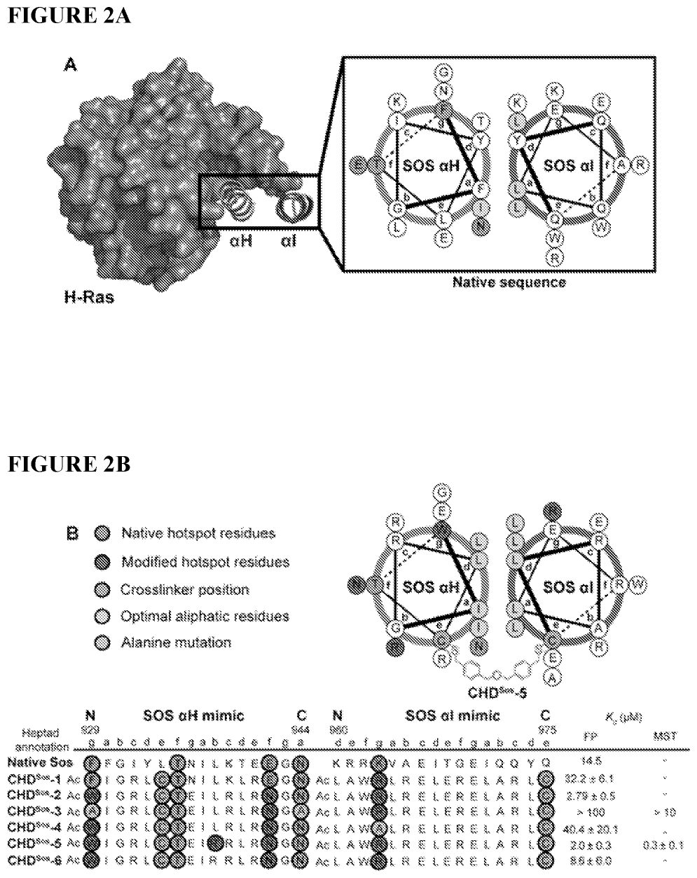

High-resolution structures of the Ras/Sos complex (PDB: 1NVW) show the catalytic region of Sos binding to Ras as a helical hairpin consisting of the αH and αI domains (see FIG. 2A). Computational alanine scanning mutagenesis suggests that the critical Ras-contacting residues, or the hot spot residues, are populated on the αH helix of the Sos hairpin (F929, T935, E942, and N944), with the αI helix potentially engaging an ionic patch, as shown in

Table 3, below (A. Patgiri, K. K. Yadav, P. S. Arora, D. Bar-Sagi, An orthosteric inhibitor of the Ras-Sos interaction. Nat. Chem. Biol. 7, 585-587 (2011); T. Kortemme, D. E. Kim, D. Baker, Computational alanine scanning of protein-protein interfaces. Sci. Signal. 2004, pl2 (2004)).

TABLE 3
Computational alanine scanning for determination of hot-spot
residues in the Ras-Sos complex (PDB 1NVW and 1BKD).
Sos αH Helix (929-944): FFGIYLTNILKTEEGN (SEQ ID NO: 35)

| | ΔΔG (kcal/mol) | ΔΔG (kcal/mol) |
|---|---|---|
| Sos Residue | from 1NVW | from 1BKD |
| F 929 | 1.64 | 1.45 |
| F 930 | 0.03 | 0.05 |
| G 931 | — | — |
| I 932 | 0.07 | 0.05 |
| Y 933 | 0.01 | — |
| L 934 | 0.65 | 0.65 |
| T 935 | 1.11 | 1.59 |
| R 936 | 0.28 | 0.83 |
| I 937 | — | — |
| L 938 | 0.63 | 0.69 |
| K 939 | 0.27 | 0.44 |
| T 940 | −0.13 | 0.05 |
| E 941 | — | — |
| E 942 | 1.10 | 0.34 |
| G 943 | — | — |
| N 944 | 2.35 | 2.63 |

CHDs that capture the conformation of αH and αI helix-loop-helix tertiary structure have been developed. Synthesis of CHDs requires an appropriately placed crosslinker at the solvent exposed surface of the two helical segments in addition to enhanced intramolecular contacts at the dimer interface. To mimic the αH/αI helix dimer, the crosslinker is placed at the ‘e’ position of the antiparallel helical construct. In prior efforts (M. G. Wuo, S. H. Hong, A. Singh, P. S. Arora, Synthetic Control of Tertiary Helical Structures in Short Peptides. J. Am. Chem. Soc. 140, 16284-16290 (2018)), the synthesis of the CHDs has been optimized by placing cysteine residues at the ‘e’ positions followed by alkylation of the thiol group with a dibenzylether crosslinker (see FIG. 2B). The native αH/αI interface features an unoptimized knob-into-hole packing; the aromatic residues at the ‘a’ and ‘d’ positions were mutated to enhance conformational stability in the minimal coiled-coil mimics (J. D. Steinkruger et al., The d′--d--d′ vertical triad is less discriminating than the a′--a--a′ vertical triad in the antiparallel coiled-coil dimer motif. J. Am. Chem. Soc. 134, 2626-2633 (2012); E. B. Hadley, O. D. Testa, D. N. Woolfson, S. H. Gellman, Preferred side-chain constellations at antiparallel coiled-coil interfaces. Proc. Natl. Acad. Sci. USA 105, 530-535 (2008); J. M. Fletcher et al., A basis set of de novo coiled-coil peptide oligomers for rational protein design and synthetic biology. ACS Synth Biol 1, 240-250 (2012)). Other non-interacting residues were also mutated to increase intra- and inter-strand salt bridge interactions and the conformational stability of the helix dimer (P. Lavigne, F. D. Sonnichsen, C. M. Kay, R. S. Hodges, Interhelical salt bridges, coiled-coil stability, and specificity of dimerization. Science 271, 1136-1137 (1996)). The designed sequences are listed in FIG. 2B and FIG. 17, and Table 2.

Sos derivatives were rationally designed with the aid two Rosetta-based computational webservers, Robetta (T. Kortemme, D. E. Kim, D. Baker, Computational alanine scanning of protein-protein interfaces. Sci. Signal. 2004, pl2 (2004)) and Rosetta Backrub(F. Lauck, C. A. Smith, G. F. Friedland, E. L. Humphris, T. Kortemme, RosettaBackrub—a web server for flexible backbone protein structure modeling and design. Nucleic Acids Res. 38, W569-575 (2010)). The Robetta server provides alanine scanning mutagenesis analysis using static models of protein interfaces and Backrub suggests potential residue substitutions to improve scores. Beyond these computational webservers, the Chimera visualization software (E. F. Pettersen et al., UCSF Chimera—A visualization system for exploratory research and analysis. Journal of Computational Chemistry 25, 1605-1612 (2004)) was utilized to predict nonnatural side chains, particularly to engage ionic patches on the protein surface. Modeling of the Ras-Sos complex and apo-Ras crystal structures suggests a potential ionic interaction between K963 residue of the αI helix and a negative patch on the Ras Switch I loop; the Sos lysine residue is sandwiched between E31 and D33 of Ras. It has been presently hypothesized that the guanidine functionality of an arginine group may engage both Ras ionic residues better than the primary amine group of a lysine side chain. Based on this hypothesis K963R substitution was inserted in the designed CHDs.

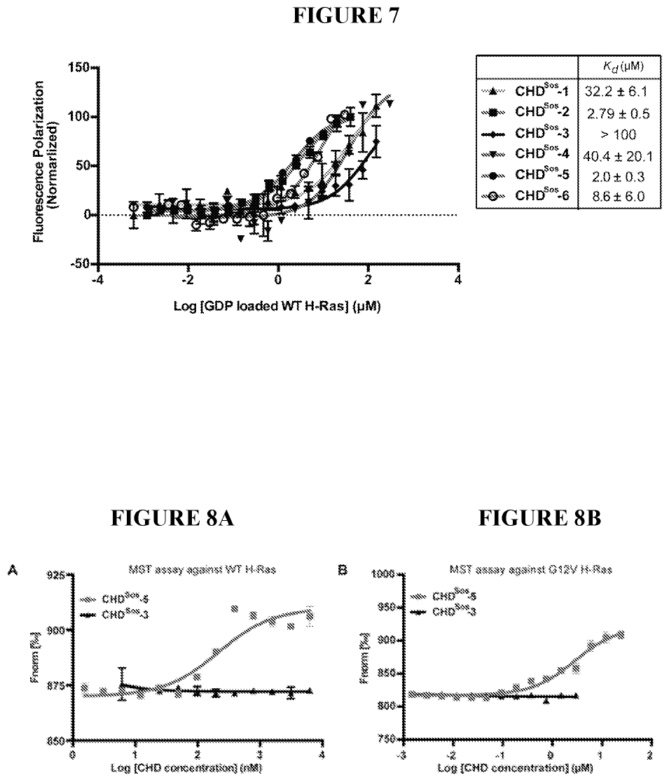

CHC^{Sos}-1 most closely mimics the native Sos hairpin sequence; the fluorescently-labeled derivative of this proteomimetic binds H-Ras with a binding affinity, K_{d}=32±6 μM, as measured in a fluorescence polarization (FP) assay with fluorescein-derivatized CHDs (see FIG. 7). In the cellular context, the binding affinity of Sos protein for Ras is highly dependent on the membrane localization of two proteins. The reported K_{d} value for the in-solution interaction of nucleotide-bound Ras with the catalytic domain of Sos is 14.5 μM (H. Sondermann et al., Structural analysis of autoinhibition in the Ras activator Son of sevenless. Cell 119, 393-405 (2004)). Therefore, the binding affinity of CHC^{Sos}-1 for Ras is in range of what is expected of a Sos fragment in the biochemical context. Computational analysis performed by Rosetta Backrub indicated that two contact residues (F929, E942) were not optimally engaged within their respective sub-pockets on the Ras surface. The analysis suggested substitutions of F929W and E942N. Substitution of these two residues in CHC^{Sos}-2 provided a 10-fold enhancement in binding affinity (K_{d}=2.8±0.5 μM). CHC^{Sos}-3 (F929A, N944A) was designed as a negative control by substituting computationally predicted critical αH binding residues with alanine. As expected, the alanine double mutant bound with a significantly reduced affinity (K_{d}>100 μM).

To analyze the importance of the ionic interactions between αI and the Ras Switch I loop, CHC^{Sos}-4 was designed, in which the single cationic arginine residue (K963R) in CHC^{Sos}-2 is substituted with an alanine. Binding analysis suggests that the ionic interactions between αI and Ras are critical for the overall complex formation as the substitution of the arginine group reduced the binding affinity to K_{d}=40±20 μM. Lastly, it was recognized that L938 on αH is situated close to a negatively charged groove in Ras; and this residue was mutated to a non-canonical homoarginine residue to potentially gain an ionic contact in CHC^{Sos}-5. CHC^{Sos}-5 binds H-Ras with a slightly improved affinity, K_{d}=2.0±0.3 μM, over CHC^{Sos}-2. The extra cationic residue in CHC^{Sos}-5 also improves its aqueous solubility as compared to CHD^{Sos}-2. CHC^{Sos}-6, which contains an arginine residues at position 938, bound Ras with a four-fold reduced affinity (K_{d}=8.6±6.0 μM) than CHC^{Sos}-5, illustrating the need for the longer homoarginine side chain at this position.

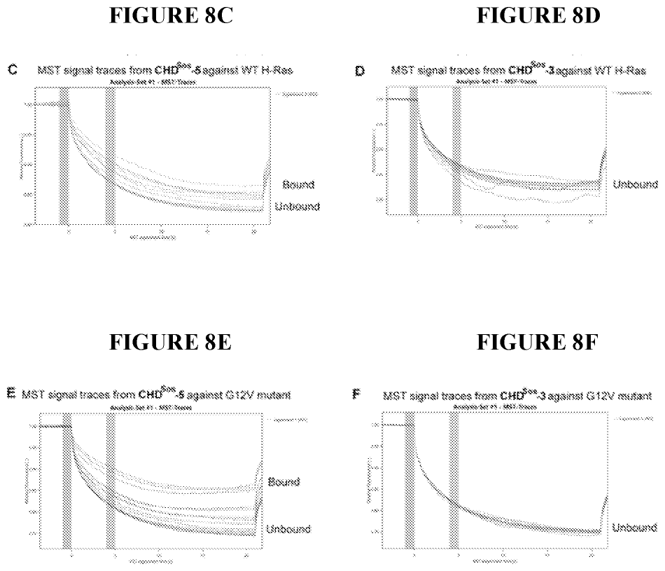

The binding affinity of the lead proteomimetic CHC^{Sos}-5 and the negative control CHC^{Sos}-3 was further analyzed with microscale thermophoresis (MST). In the MST assay, CHC^{Sos}-5 binds fluorescently-labeled Ras with nanomolar binding affinity (K_{d}=0.3±0.1 μM, see FIG. 2B, FIG. 8; as in with the polarization assay, the alanine mutant CHC^{Sos}-3 binds poorly to the protein target (K_{d}>100 μM). The improved binding affinities observed with MST suggest that the dye placement may not be optimal in the fluorescence polarization assay (N. J. Moerke, Fluorescence Polarization (FP) Assays for Monitoring Peptide-Protein or Nucleic Acid-Protein

Binding. Curr. Protoc. Chem. Biol. 1, 1-15 (2009)).

### Example 23: Sos Proteomimetic CHC^{Sos}-5 is Conformationally and Proteolytically Stable and Binds Ras in its Dynamic Switch Region

The conformational stability of the Sos proteomimetic CHC^{Sos}-5 was assessed

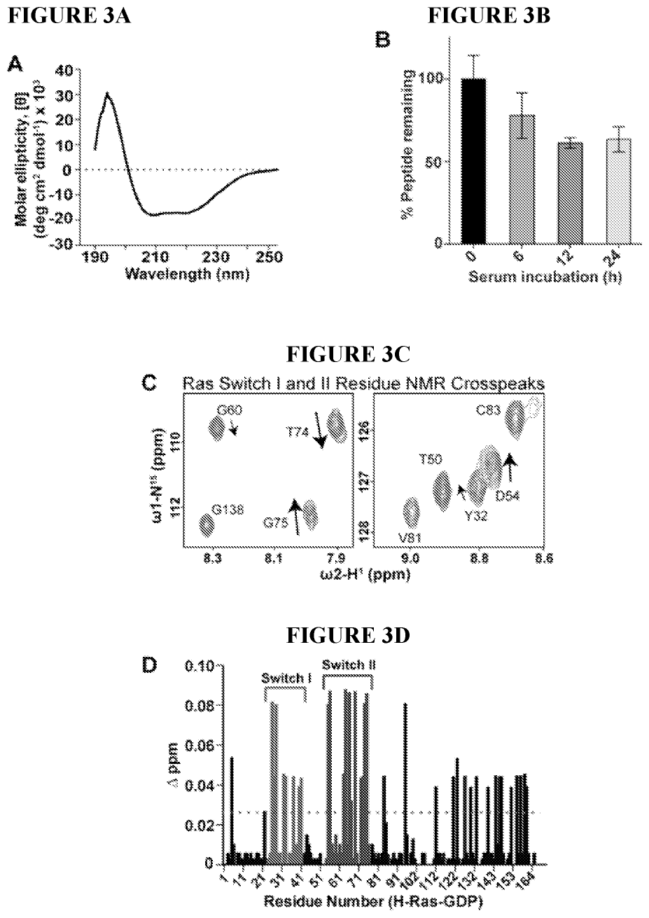

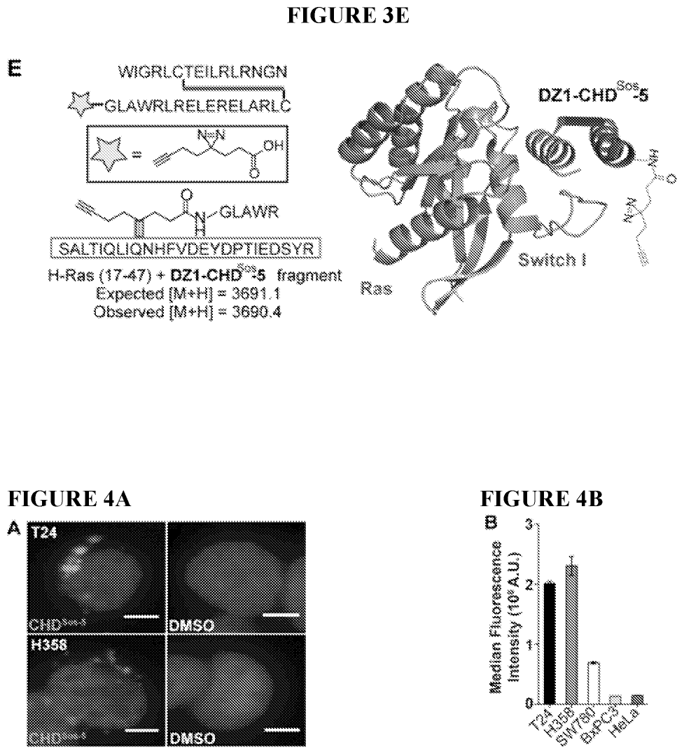

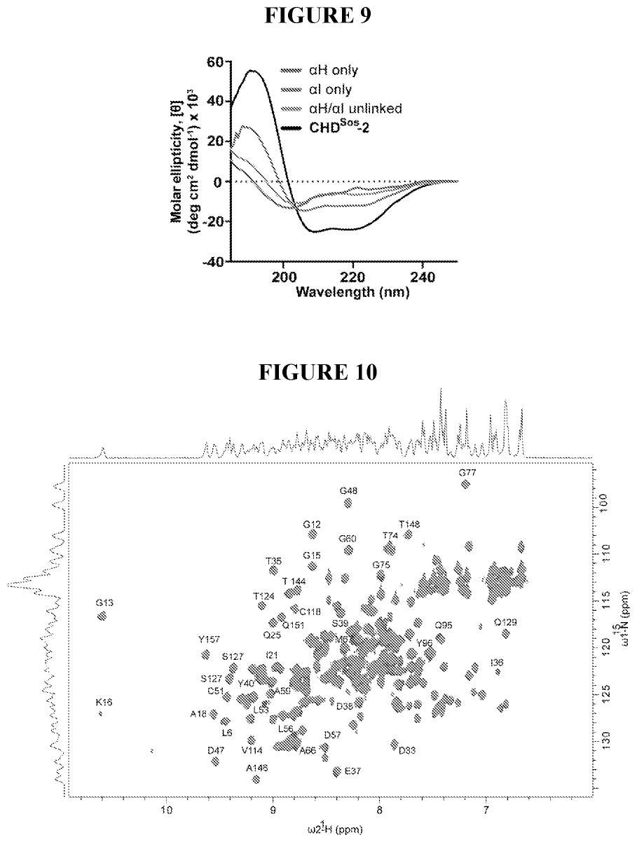

with circular dichroism (CD) spectroscopy. CD provides a unique signature for α-helices with a local maximum at 195 nm and local minima at 208 and 222 nm (N. R. Kallenbach, P. C. Lyu, H. X. Zhou, “CD spectroscopy and the helix-coil transition in peptides and polypeptides” in Circular Dichroism and the Conformational Analysis of Biomolecules, G. D. Fasman, Ed. (Plenum Press, New York, 1996); M. C. Manning, R. W. Woody, Theoretical Cd Studies of Polypeptide Helices—Examination of Important Electronic and Geometric Factors. Biopolymers 31, 569-586 (1991)). CD experiments indicate that CHC^{Sos}-5 has a well-defined helical character (FIG. 3A). In contrast, the individual peptides corresponding to the Sos αH and αI display a random coil-like signature, respectively (FIG. 9). Equimolar titrations of the unlinked peptides revealed no defined structure, while the crosslinked CHC^{Sos}-2 displayed prominent helical characteristics.

Enzymatic proteolysis is a critical factor limiting the potential of peptide therapeutics. The crosslinked helix dimers have shown resistance to proteolytic degradation due to their conformational stability (J. Sadek et al., Modulation of virus-induced NF-kappaB signaling by NEMO coiled coil mimics. Nat Commun 11, 1786 (2020)). The proteolytic stability of CHC^{Sos}-5 was analyzed in serum. The rate of proteolysis of CHC^{Sos}-5 was determined in an HPLC-based time-course assay under ex vivo conditions. CHC^{Sos}-5 exhibits considerable tolerance to proteolysis by serum proteases with a calculated t_{1/2}>24 h. Approximately 60% of the proteomimetic remains intact after 24 h (FIG. 3B).

The MST and fluorescence polarization binding data indicate that CHC^{Sos}-5 binds Ras with high nanomolar to low micromolar binding affinity. To determine if the proteomimetic engaged Ras at the Sos-binding region, as designed, a titration heteronuclear single quantum coherence NMR spectroscopy (HSQC) experiment, which monitors chemical shift changes of specific protein residues upon ligand binding or conformation change was conducted. Increasing concentrations (2.5 and 5 eq.) of CHC^{Sos}-5 to ¹⁵N-labeled H-Ras (100 μM) led to changes in peak shifts that correspond to residues within the Ras Switch I and II regions (FIG. 3C, FIG. 10). FIG. 3D displays the observed chemical shift changes after CHC^{Sos}-5 titration (5 eq.) as a bar graph.

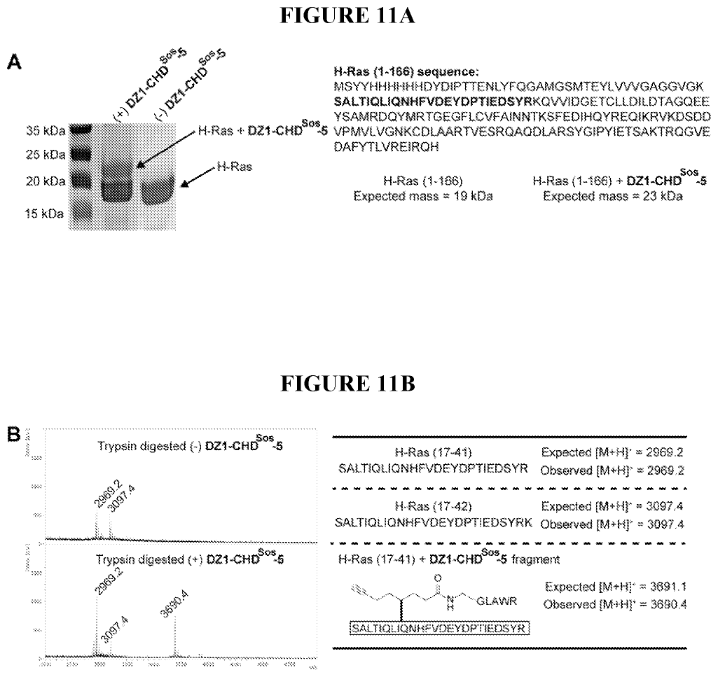

To further confirm the binding site occupancy, CHC^{Sos}-5 was elaborated with a diazirine moiety as a photo-triggered crosslinking group at the N-terminus of the αI-helix (FIG. 3E). It was hypothesized that upon irradiation of diazirine, the resultant reactive carbene would covalently label the Ras hinge region if the association occurred at the Sos binding site. H-Ras was incubated with 5 eq. DZ1-CHC^{Sos}-5, and the complex was exposed to UV light. Agarose gel electrophoresis revealed a distinct new monolabeled protein band. The trypsin treated protein was analyzed by mass spectroscopy to reveal the crosslinked Ras fragment. A fragment mass of digested DZ1-CHC^{Sos}-5 photo-crosslinked with the Ras hinge region was observed (FIG. 3E, FIG. 11). Both the titration HSQC and the chemical crosslinking results indicate that the designed proteomimetic binds to the Sos binding surface of Ras.

### Example 24: CHC^{Sos}-5 Exhibits Selective Cellular Penetration in Mutant Ras Cancer Cells

The in vitro results suggest that the designed CHDs may modulate Ras signaling. Effective cellular modulation requires efficient uptake and cytoplasmic localization of the compounds into the cell. Mechanisms of peptidomimetic transport into cancer cells have been recently comprehensively analyzed. It was observed that efficient uptake of conformationally constrained peptidomimetics is directly correlated with the macropinocytic activity of each cell line regardless of size, charge, and conformation of the peptidomimetic (D. Y. Yoo et al., Macropinocytosis as a Key Determinant of Peptidomimetic Uptake in Cancer Cells. J. Am. Chem. Soc. 142, 14461-14471 (2020); C. Commisso et al., Macropinocytosis of protein is an amino acid supply route in Ras-transformed cells. Nature 497, 633-637 (2013)).

In particular, CHDs exhibited high levels of cellular uptake and endosomal escape into the cytoplasm in macropinocytic cells despite their higher molecular weight - the uptake of CHDs was similar to that of Tat, a polycationic, cell-penetrating peptide known for high cellular internalization (P. Lönn et al., Enhancing Endosomal Escape for Intracellular Delivery of Macromolecular Biologic Therapeutics. Scientific Reports 6, 32301 (2016); P. Lönn, S. F. Dowdy, Cationic PTD/CPP-mediated macromolecular delivery: charging into the cell. Expert Opinion on Drug Delivery 12, 1627-1636 (2015)).

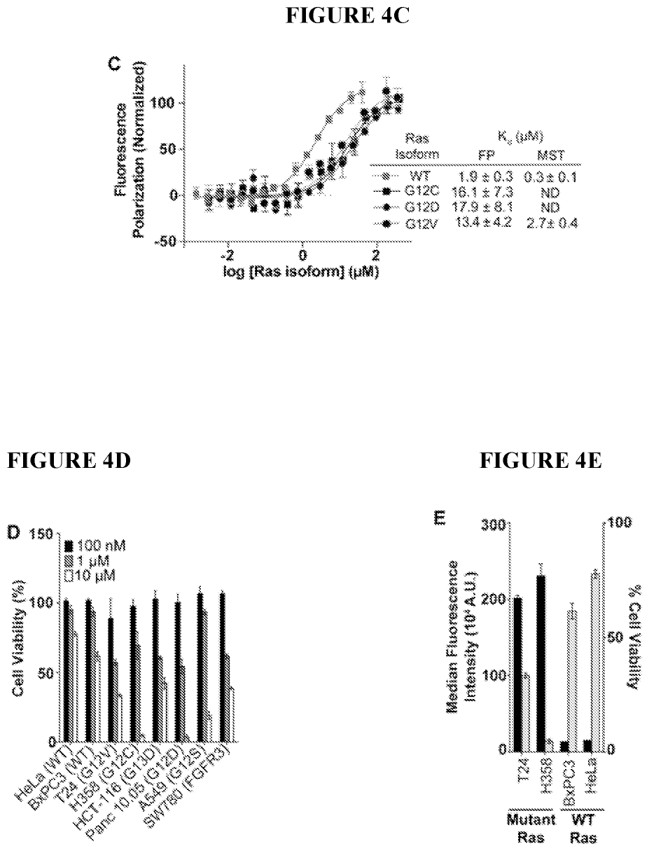

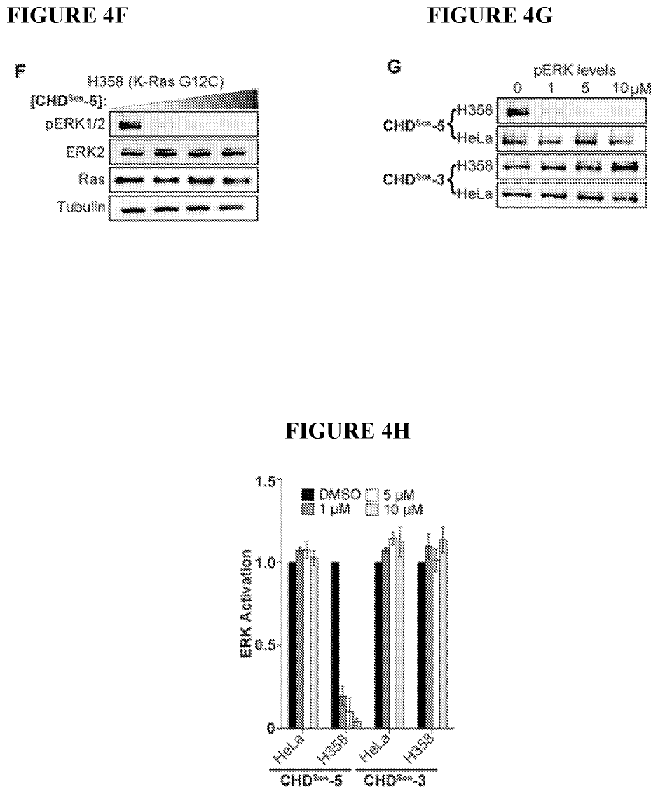

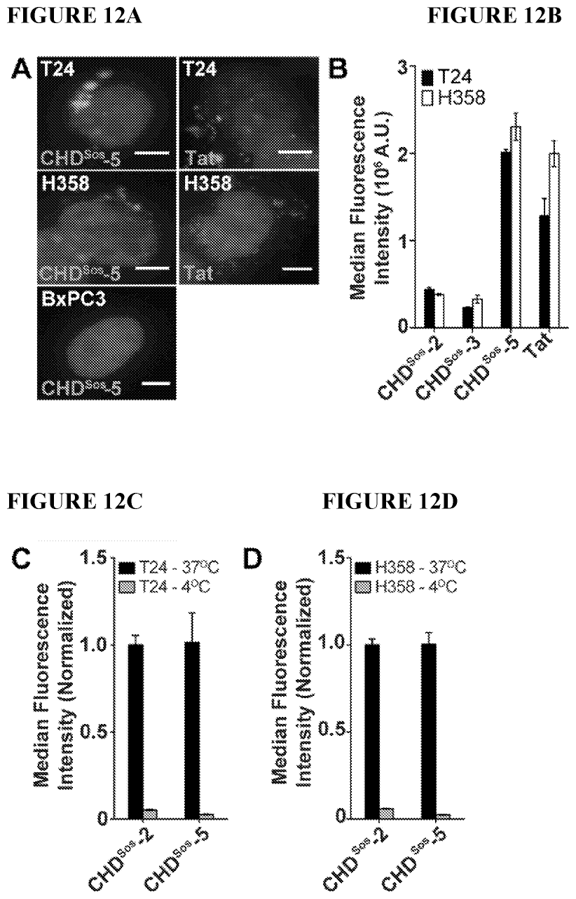

Macropinocytosis may be upregulated in cells with activating mutations in Ras or other endemic mutations within the Ras pathway. In keeping with these earlier analyses, live cell fluorescence microscopy showed significant cellular uptake of fluorescein-labeled CHC^{Sos}-5 into the cytosol in the Ras mutant T24 (H-Ras G12V) bladder and H358 (K-Ras G12C) lung cancer cells (FIG. 4A). The intracellular intensity of fluorescent CHC^{Sos}-5 is similar to that of fluorescently-labeled Tat peptide (FIG. 12A). To support the microscopy studies, peptide uptake was quantified using flow cytometry analysis and observed similar results for CHC^{Sos}-5 and Tat.

Negative controls CHC^{Sos}-2 and CHC^{Sos}-3 exhibited lower uptake than CHC^{Sos}-5 presumably due to the removal of an arginine residue (FIG. 4B, FIG. 12B). As expected, low cellular uptake was observed for peptides in HeLa and BxPC3 cells, both of which have a low macropinocytosis activity. When compared to cells that were incubated at 37° C., cold-treated T24 and H358 cells showed >90% reduction in uptake for fluorescently tagged CHC^{Sos}-2 and CHC^{Sos}-5 (FIG. 12C-D), which should disrupt energy-dependent uptake pathways including macropinocytosis. Enhanced macropinocytotic activity is not limited to cells with Ras mutations and certain other mutations are also known to upregulate this activity. For example, CHC^{Sos}-5 is also permeable in SW780 cells, which contain oncogenic FGFR3 fusions (FIG. 4B). Overall, reliance of cellular permeability on certain cancer mutations suggests that certain cancers may be more amenable to therapeutic proteomimetics.

### Example 25: CHC^{Sos}-5 Binds Wild-Type and Mutant Isoforms of Ras

The enhanced cellular uptake of CHC^{Sos}-5 in cancer cells with mutated forms of Ras suggests that the Sos proteomimetic may be selectively toxic to these cells. Based on this premise, the potential of CHC^{Sos}-5 to engage the mutant forms of H-Ras was explored, specifically where G12 is mutated to cysteine, aspartic acid and valine (G12C, G12D and G12V). Substitution of the glycine residue with polar and β-branched residues modulates the conformation of the dynamic switch regions between the “open” and “closed” Ras forms, and these mutations are often observed in the oncogenic Ras isoforms (S. Lu, H. Jang, R. Nussinov, J. Zhang, The Structural Basis of Oncogenic Mutations G12, G13 and Q61 in Small GTPase K-Ras4B. Scientific Reports 6, 21949 (2016)). The mutants were expressed in the H-Ras construct for in vitro binding analyses. Activating mutations are more often found in the K-Ras isoform in human cancers, but the Sos-binding surface of H-Ras and K-Ras are fully conserved with differences largely localized to the C-terminal hypervariable region, which is implicated in membrane anchoring of Ras proteins (E. Castellano, E. Santos, Functional Specificity of Ras Isoforms: So Similar but So Different. Genes & Cancer 2, 216-231 (2011)). Titration of fluorescein-derivatized CHC^{Sos}-5 with wild-type and G12X mutant H-Ras proteins reveals that the proteomimetic binds all Ras proteins within a similar low micromolar affinity range—albeit with a preference for the wild-type form (FIG. 4C). The binding affinity of CHC^{Sos}-5 for wild-type, G12C, G12D, and G12V H-Ras proteins is expected to translate to wild type and mutant forms of K-Ras.

### Example 26: CHC^{Sos}-5 is Selectively Toxic to Mutant Ras Cancer Cells by Downregulating Ras Signaling

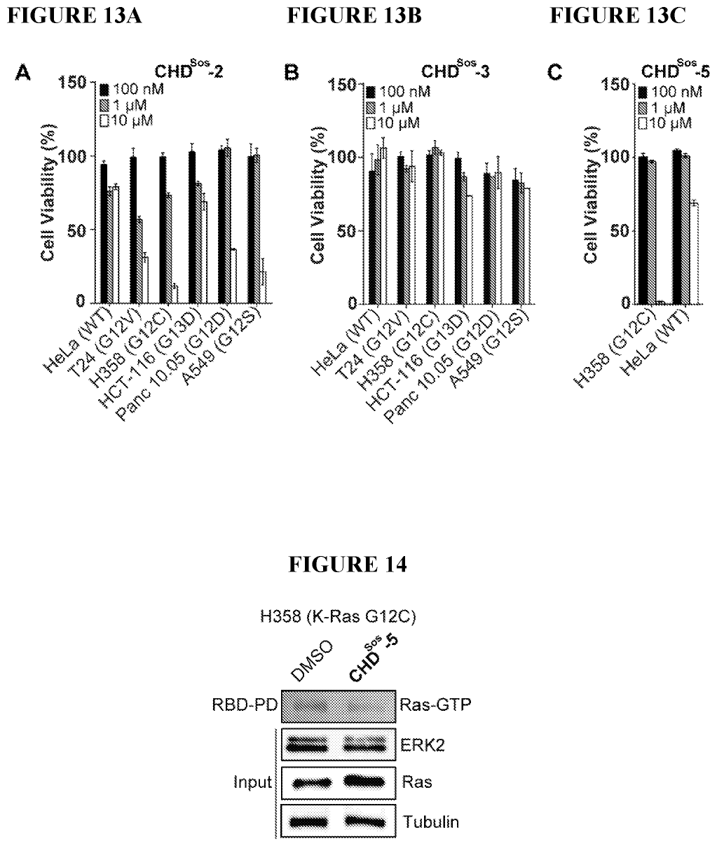

Encouraged by the in vitro results, which showed that CHC^{Sos}-5 can bind wild-type and mutant Ras forms with micromolar affinity and that CHC^{Sos}-5 has selectively high permeability in Ras mutant cell lines, the potential of the Sos proteomimetic to inhibit Ras signaling in cells was evaluated. The toxicity of the Sos proteomimetic using the MTT cell viability assay was assessed. In preliminary studies, it was observed that CHC^{Sos}-5 can function in the presence of serum. This result is important because cells often need to be treated with peptides under artificial serum-free conditions to assess their cellular potential because peptides may be retained in the medium by the hydrophobic components present in serum. As expected from the cell permeability results, CHC^{Sos}-2 and CHC^{Sos}-5 exhibited concentration-dependent toxicity against cell lines containing oncogenic Ras mutations in comparison to the wild-type Ras HeLa control cell line (FIG. 4D, FIG. 13A). Cell viability was shown to be inversely correlated to the inherent macropinocytic uptake level between different cell lines (FIG. 4E). The results suggest that exploitation of upregulated macropinocytosis presents a potentially unexploited advantage for delivering therapeutics to mutant cancer cells. Importantly, the alanine control CHC^{Sos}-3 displayed little to no effect on the viability of the tested cell lines, suggesting that targeting of Ras is leading to cellular toxicity (FIG. 13B). The MTT assay results were validated with a second cell viability assay based on the CellTiter-Glo Luminescent system, which confirmed the selective toxicity of CHC^{Sos}-5 against a mutant Ras (H358) cell line relative to the wild-type Ras control (HeLa) (FIG. 13C).

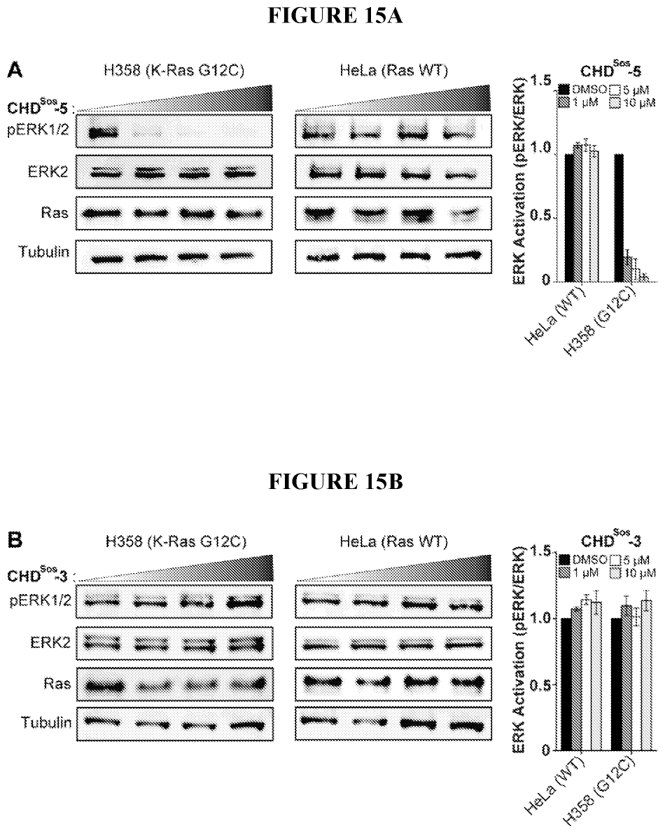

To demonstrate that the cellular toxicity of CHC^{Sos}-5 is correlated with downregulation of Ras signaling, the impact of the Sos proteomimetic on the cellular concentrations of GTP-bound Ras and phosphorylated ERK was probed. It was first determined if treatment of cells with CHC^{Sos}-5 downregulates Ras activation. Cellular levels of Ras-GTP were assessed with a Raf1 Ras-binding domain (RBD) pulldown assay (S. J. Taylor, R. J. Resnick, D. Shalloway, “Nonradioactive determination of Ras-GTP levels using activated ras interaction assay” in Methods in Enzymology. (Academic Press, 2001), vol. 333, pp. 333-342). The extent of Ras phosphorylation was significantly reduced in the presence of 10 μM CHC^{Sos}-5 in H358 cells (FIG. 14). Ras is a critical mediator of multiple signal transduction pathways, and ERK activation is a well-studied node in the Ras effector pathway (R. Garcia-Gomez, X. R. Bustelo, P. Crespo, Protein-Protein Interactions: Emerging Oncotargets in the RAS-ERK Pathway. Trends Cancer 4, 616-633 (2018)). To determine if the impact of CHC^{Sos}-5 on Ras-GTP levels leads to the intended decrease in ERK phosphorylation, H358 and HeLa cells in complete medium were treated with increasing concentrations of CHC^{Sos}-3 or CHC^{Sos}-5. The resulting lysates were blotted for phosphorylated ERK. CHC^{Sos}-5 significantly reduced ERK activation levels (A. A. Samatar, P. I. Poulikakos, Targeting RAS-ERK signalling in cancer: promises and challenges. Nat. Rev. Drug Discov. 13, 928-942 (2014)), IC₅₀<1 μM, in a concentration dependent manner in the K-Ras G12C mutant cell line while exerting little effect in HeLa cells. This differential activity is consistent with the cellular permeability differences of the proteomimetic between the two cell lines (FIG. 4F-H, FIG. 15A). As expected, the alanine control CHC^{Sos}-3 did not suppress ERK phosphorylation in either cell line (FIG. 4G-H, FIG. 15B). The observed downregulation of activated ERK with CHC^{Sos}-5 treatment is consistent with the intracellular engagement of Ras by the proteomimetic.

### Example 27: Chemoproteomics Analysis Reveals Cellular Targets of CHC^{Sos}-5

The Ras superfamily of small GTPases consists of over 150 members and includes the Ras, Rho, Rab, Arf and Ran subfamilies (D. Vigil, J. Cherfils, K. L. Rossman, C. J. Der, Ras superfamily GEFs and GAPs: validated and tractable targets for cancer therapy? Nat. Rev. Cancer 10, 842-857 (2010)). The superfamily has high structural and sequence conservation in the GTP/GDP nucleotide-binding domain which is engaged by various structurally conserved GEFs. The Ras-Sos complex formation is promoted by membrane localization of both proteins but it is not known how many Ras family members Sos may engage if membrane recruitment was not a determining factor. The designed Sos proteomimetic, CHC^{Sos}-5, mimics a portion of the Sos nucleotide binding domain and does not contain a membrane anchor. It was hypothesized that CHC^{Sos}-5 would likely have multiple cellular partners and a chemoproteomics analysis may reveal its major targets.

To identify putative binding partners of CHC^{Sos}-5, H358 lung cancer cells were subjected to photoaffinity labeling followed by enrichment of the interactors and their characterization by established mass spectroscopy-based proteomics protocols (C. G. Parker, M.

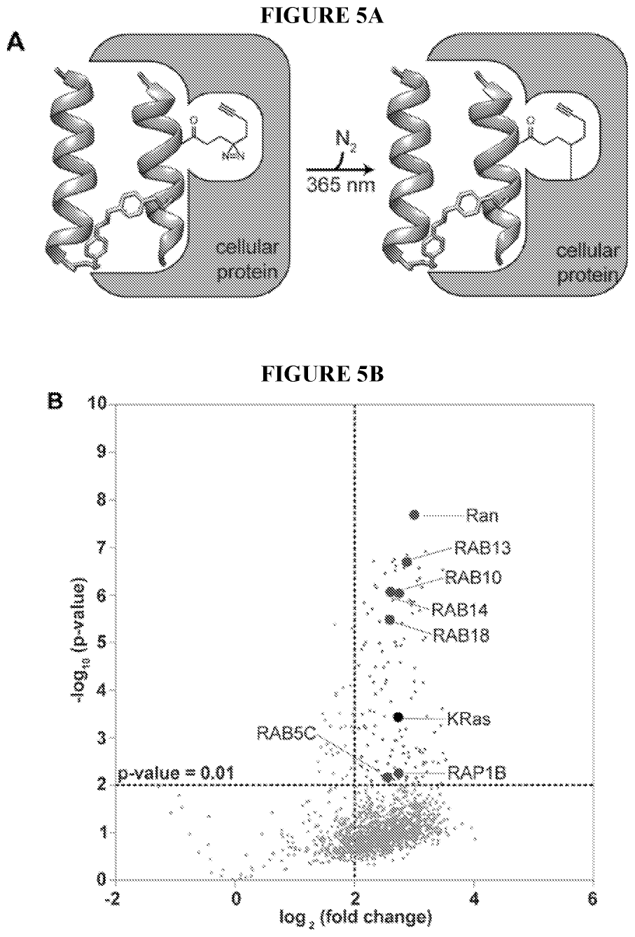

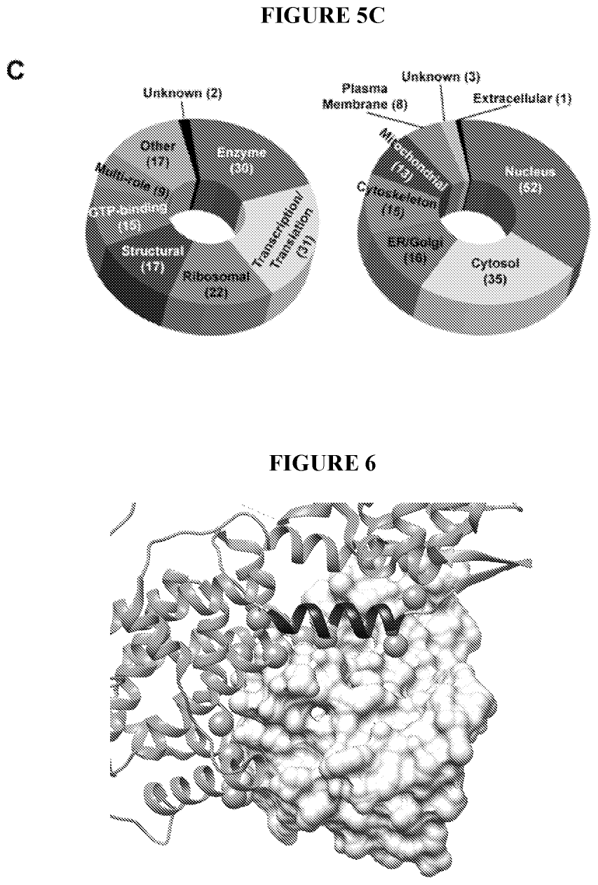

R. Pratt, Click Chemistry in Proteomic Investigations. Cell 180, 605-632 (2020)). Labeling was accomplished using a variant of CHC^{Sos}-5 with a photolabile diazirine crosslinker attached in place of the non-natural homoarginine residue (DZ2-CHC^{Sos}-5, FIG. 5A).

Separate populations of cells were treated with DZ2-CHC^{Sos}-5 (10 μM) alongside a previously described negative control diazirine probe (CP-2-66, Table 2, above) (C. G. Parker et al., Ligand and Target Discovery by Fragment-Based Screening in Human Cells. Cell 168, 527-541.e₅₂₉ (2017)) and then exposed to UV light (365 nm) to induce photocrosslinking of DZ2-CHC^{Sos}-5 bound protein targets. Cells were lysed, and the probe-labeled proteins were conjugated to biotin azide via CuAAC, enriched with streptavidin-coated resin and trypsinized as previously described in Y. Wang et al., Expedited mapping of the ligandable proteome using fully functionalized enantiomeric probe pairs. Nat. Chem. 11, 1113-1123 (2019). Subsequently, tryptic peptides from each population were labeled with isobaric tandem mass tags (TMT) (G. C. McAlister et al., MultiNotch MS3 Enables Accurate, Sensitive, and Multiplexed Detection of Differential Expression across Cancer Cell Line Proteomes. Anal. Chem. 86, 7150-7158 (2014)) to enable quantitative comparisons and analyzed by mass spectrometry (MS)-based proteomics. Proteins were considered targets if they were enriched by an average value of >4-fold across biological duplicate experiments (p <0.01; Table 2, above). Overall, 143 protein targets were identified, amongst these, K-Ras was shown to be enriched by DZ2-CHC^{Sos}-5 along with seven other members of the Ras GTPase superfamily: the GTP-binding nuclear protein (RAN) and several Ras-related proteins (RAB13, RAB10, RAB14, RAB18, RAB5C, RAP1B) (FIG. 5B).

TABLE 4A
Analysis of sequence similarity within enriched
Ras GTPase proteins as compared to K-Ras.

| | | | | Switch I + | | | |
|---|---|---|---|---|---|---|---|
| | | G-Domain | | Binding Helix | | Switch II | |
| | | (Residues 1-166) | | (Residues 10-40) | | (Residues 56-75) | |
| | Gene | % | % | % | % | % | % |
| Accession | Symbol | Identity | Similarity | Identity | Similarity | Identity | Similarity |
| P01116-2 | KRAS | — | — | — | — | — | — |
| P62826 | RAN | 25.9 | 68.1 | 25.8 | 67.7 | 45 | 75 |
| P51153 | RAB13 | 38.0 | 75.3 | 35.5 | 71.0 | 40 | 70 |
| P61026 | RAB10 | 35.5 | 73.5 | 25.8 | 64.5 | 40 | 70 |
| P61106 | RAB14 | 31.3 | 69.9 | 35.5 | 64.5 | 45 | 70 |
| Q9NP72 | RAB18 | 33.7 | 71.1 | 35.5 | 64.5 | 40 | 70 |
| P61224 | RAP1B | 57.2 | 88.0 | 74.2 | 96.8 | 65 | 90 |
| P51148 | RAB5C | 31.9 | 72.9 | 29.0 | 77.4 | 45 | 65 |
| P01112 | HRAS | 94.0 | 98.8 | 100 | 100 | 100 | 100 |
| P01111 | NRAS | 94.0 | 98.2 | 100 | 100 | 100 | 100 |

TABLE 4B
Analysis of sequence similarity within enriched
non-Ras G-proteins as compared to K-Ras.

| | | | | Switch I + | | | |
|---|---|---|---|---|---|---|---|
| | | G-Domain | | Binding Helix | | Switch II | |
| | | (Residues 1-166) | | (Residues 10-40) | | (Residues 56-75) | |
| | Gene | % | % | % | % | % | % |
| Accession | Symbol | Identity | Similarity | Identity | Similarity | Identity | Similarity |
| P01116-2 | KRAS | — | — | — | 1 | — | — |
| Q5JWF2 | GNAS | 41 | 62 | 10 | 8 | 4 | 6 |
| P36404 | ARL2 | 23.0 | 58.0 | 6.0 | 15.0 | 4 | 9 |
| Q9BX10 | GTPBP2 | 13.3 | 44.0 | 22.6 | 54.8 | 20 | 40 |
| P19784 | CSNK2A2 | 21.1 | 52.4 | 6.5 | 12.9 | 10 | 15 |
| Q9BVP2 | GNL3 | 19.9 | 49.4 | 22.6 | 51.6 | 5 | 25 |
| O60841 | EIF5B | 22.3 | 56.6 | 25.8 | 58.1 | 30 | 65 |
| Q9NTK5 | OLA1 | 24.1 | 60.2 | 29.0 | 58.1 | 15 | 55 |

Tables 4A and 4B compare sequences of Ras-related GTPases and other G-proteins enriched from proteomics analysis to human K-Ras. Each sequence was aligned and compared to K-Ras within the G-domain (aa 1-166) and the Sos &H/al hairpin binding region (aa 10-40, 56-75). Sequence identity assesses the degree of fully conserved residues within the indicated regions, while sequence similarity refers to variable residue substitutions with similar chemical properties according to the Gonnet PAM 250 matrix (G. Gonnet, M. Cohen, S. Benner, Exhaustive matching of the entire protein sequence database. Science 256, 1443-1445 (1992)).

The analysis suggests that there is a high degree of sequence similarity (50-70%) between the enriched Ras-related and other G-proteins in the Sos helix hairpin binding region (J. Colicelli, Human RAS Superfamily Proteins and Related GTPases. Sci. Signal. 2004, rel3-rel3 (2004)).

The enriched targets identified by the photoaffinity labeling method include proteins with a wide array of functions (FIG. 5C). A majority of these interactors is localized within the intracellular compartment of the cell, which supports the hypothesis that CHC^{Sos}-5 avoids endosomal entrapment upon internalization (FIG. 5C). The biological impact of targeting other GTPases, beyond Ras, and non-Ras family proteins with the Sos proteomimetic remains to be determined.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

As various changes can be made in the above-described subject matter without departing from the scope and spirit of the present invention, it is intended that all subject matter contained in the above description, or defined in the appended claims, be interpreted as descriptive and illustrative of the present invention. Many modifications and variations of the present invention are possible in light of the above teachings. Accordingly, the present description is intended to embrace all such alternatives, modifications, and variances which fall within the scope of the appended claims.

All patents, applications, publications, test methods, literature, and other materials cited herein are hereby incorporated by reference in their entirety as if physically present in this specification.

### REFERENCES

1. P. A. Konstantinopoulos, M. V. Karamouzis, A. G. Papavassiliou, Post-translational modifications and regulation of the RAS superfamily of GTPases as anticancer targets. Nat. Rev. Drug Discov. 6, 541-555 (2007).
2. P. A. Boriack-Sjodin, S. M. Margarit, D. Bar-Sagi, J. Kuriyan, The structural basis of the activation of Ras by Sos. Nature 394, 337-343 (1998).
3. A. R. Moore, S. C. Rosenberg, F. McCormick, S. Malek, RAS-targeted therapies: is the undruggable drugged? Nature Reviews Drug Discovery 19, 533-552 (2020).
4. H. Chen, J. B. Smaill, T. Liu, K. Ding, X. Lu, Small-Molecule Inhibitors Directly Targeting KRAS as Anticancer Therapeutics. Journal of Medicinal Chemistry 63, 14404-14424 (2020).
5. J. Canon et al., The clinical KRAS(G12C) inhibitor AMG 510 drives anti-tumour immunity. Nature 575, 217-223 (2019).
6. J. M. Ostrem, U. Peters, M. L. Sos, J. A. Wells, K. M. Shokat, K-Ras(G12C) inhibitors allosterically control GTP affinity and effector interactions. Nature 503, 548-551 (2013).
7. Z. Zhang et al., GTP-State-Selective Cyclic Peptide Ligands of K-Ras(G12D) Block Its Interaction with Raf. ACS Central Science 6, 1753-1761 (2020).
8. K. Sakamoto, T. Masutani, T. Hirokawa, Generation of KS-58 as the first K-Ras(G12D)-inhibitory peptide presenting anti-cancer activity in vivo. Scientific Reports 10, 21671 (2020).
9. D. Y. Yoo, A. D. Hauser, S. T. Joy, D. Bar-Sagi, P. S. Arora, Covalent Targeting of Ras G12C by Rationally Designed Peptidomimetics. ACS Chem. Biol. 15, 1604-1612 (2020).
10. A. Patgiri, K. K. Yadav, P. S. Arora, D. Bar-Sagi, An orthosteric inhibitor of the Ras-Sos interaction. Nat. Chem. Biol. 7, 585-587 (2011).
11. B. N. Kholodenko, J. F. Hancock, W. Kolch, Signalling ballet in space and time. Nature Reviews Molecular Cell Biology 11, 414-426 (2010).
12. Y. S. Chang et al., Stapled alpha-helical peptide drug development: a potent dual inhibitor of MDM2 and MDMX for p53-dependent cancer therapy. Proc. Natl. Acad. Sci. USA 110, E3445-3454 (2013).
13. S. Kushal et al., Protein domain mimetics as in vivo modulators of hypoxia-inducible factor signaling. Proc. Natl. Acad. Sci. USA 110, 15602-15607 (2013).
14. S. Liu, R. W. Cheloha, T. Watanabe, T. J. Gardella, S. H. Gellman, Receptor selectivity from minimal backbone modification of a polypeptide agonist. Proc. Natl. Acad. Sci. USA 115, 12383-12388 (2018).
15. M. Pelay-Gimeno, A. Glas, O. Koch, T. N. Grossmann, Structure-Based Design of Inhibitors of Protein-Protein Interactions: Mimicking Peptide Binding Epitopes. Angew. Chem. Int. Ed. 54, 8896-8927 (2015).
16. W. S. Horne, T. N. Grossmann, Proteomimetics as protein-inspired scaffolds with defined tertiary folding patterns. Nat. Chem. 12, 331-337 (2020).
17. H. Adihou et al., A protein tertiary structure mimetic modulator of the Hippo signalling pathway. Nat. Commun. 11, 5425 (2020).
18. J. Sadek et al., Modulation of virus-induced NF-kappaB signaling by NEMO coiled coil mimics. Nat Commun 11, 1786 (2020).
19. A. M. Watkins, M. G. Wuo, P. S. Arora, Protein-Protein Interactions Mediated by Helical Tertiary Structure Motifs. J. Am. Chem. Soc. 137, 11622-11630 (2015).
20. M. G. Wuo, S. H. Hong, A. Singh, P. S. Arora, Synthetic Control of Tertiary Helical Structures in Short Peptides. J. Am. Chem. Soc. 140, 16284-16290 (2018).
21. M. G. Wuo, A. B. Mahon, P. S. Arora, An Effective Strategy for Stabilizing Minimal Coiled Coil Mimetics. J. Am. Chem. Soc. 137, 11618-11621 (2015).
22. F. Lauck, C. A. Smith, G. F. Friedland, E. L. Humphris, T. Kortemme, RosettaBackrub—a web server for flexible backbone protein structure modeling and design. Nucleic Acids Res. 38, W569-575 (2010).
23. D. Rooklin et al., Targeting Unoccupied Surfaces on Protein-Protein Interfaces. J. Am. Chem. Soc. 139, 15560-15563 (2017).
24. T. Kortemme, D. E. Kim, D. Baker, Computational alanine scanning of protein-protein interfaces. Sci. Signal. 2004, pl2 (2004).
25. J. D. Steinkruger et al., The d′--d--d′ vertical triad is less discriminating than the a′ -- a -- a′ vertical triad in the antiparallel coiled-coil dimer motif. J. Am. Chem. Soc. 134, 2626-2633 (2012).
26. E. B. Hadley, O. D. Testa, D. N. Woolfson, S. H. Gellman, Preferred side-chain constellations at antiparallel coiled-coil interfaces. Proc. Natl. Acad. Sci. USA 105, 530-535 (2008).
27. J. M. Fletcher et al., A basis set of de novo coiled-coil peptide oligomers for rational protein design and synthetic biology. ACS Synth Biol 1, 240-250 (2012).
28. P. Lavigne, F. D. Sonnichsen, C. M. Kay, R. S. Hodges, Interhelical salt bridges, coiled-coil stability, and specificity of dimerization. Science 271, 1136-1137 (1996).
29. E. F. Pettersen et al., UCSF Chimera—A visualization system for exploratory research and analysis. Journal of Computational Chemistry 25, 1605-1612 (2004).
30. H. Sondermann et al., Structural analysis of autoinhibition in the Ras activator Son of sevenless. Cell 119, 393-405 (2004).
31. N. J. Moerke, Fluorescence Polarization (FP) Assays for Monitoring Peptide-Protein or Nucleic Acid-Protein Binding. Curr. Protoc. Chem. Biol. 1, 1-15 (2009).
32. N. R. Kallenbach, P. C. Lyu, H. X. Zhou, “CD spectroscopy and the helix-coil transition in peptides and polypeptides” in Circular Dichroism and the Conformational Analysis of Biomolecules, G. D. Fasman, Ed. (Plenum Press, New York, 1996).
33. M. C. Manning, R. W. Woody, Theoretical Cd Studies of Polypeptide Helices—Examination of Important Electronic and Geometric Factors. Biopolymers 31, 569-586 (1991).
34. D. Y. Yoo et al., Macropinocytosis as a Key Determinant of Peptidomimetic Uptake in Cancer Cells. J. Am. Chem. Soc. 142, 14461-14471 (2020).
35. C. Commisso et al., Macropinocytosis of protein is an amino acid supply route in Ras-transformed cells. Nature 497, 633-637 (2013).
36. P. Lönn et al., Enhancing Endosomal Escape for Intracellular Delivery of Macromolecular Biologic Therapeutics. Scientific Reports 6, 32301 (2016).
37. P. Lönn, S. F. Dowdy, Cationic PTD/CPP-mediated macromolecular delivery: charging into the cell. Expert Opinion on Drug Delivery 12, 1627-1636 (2015).
38. S. Lu, H. Jang, R. Nussinov, J. Zhang, The Structural Basis of Oncogenic Mutations G12, G13 and Q61 in Small GTPase K-Ras4B. Scientific Reports 6, 21949 (2016).
39. E. Castellano, E. Santos, Functional Specificity of Ras Isoforms: So Similar but So Different. Genes & Cancer 2, 216-231 (2011).
40. S. J. Taylor, R. J. Resnick, D. Shalloway, “Nonradioactive determination of Ras-GTP levels using activated ras interaction assay” in Methods in Enzymology. (Academic Press, 2001), vol. 333, pp. 333-342.
41. R. Garcia-Gomez, X. R. Bustelo, P. Crespo, Protein-Protein Interactions: Emerging Oncotargets in the RAS-ERK Pathway. Trends Cancer 4, 616-633 (2018).
42. A. A. Samatar, P. I. Poulikakos, Targeting RAS-ERK signalling in cancer: promises and challenges. Nat. Rev. Drug Discov. 13, 928-942 (2014).
43. D. Vigil, J. Cherfils, K. L. Rossman, C. J. Der, Ras superfamily GEFs and GAPs: validated and tractable targets for cancer therapy? Nat. Rev. Cancer 10, 842-857 (2010).
44. C. G. Parker, M. R. Pratt, Click Chemistry in Proteomic Investigations. Cell 180, 605-632 (2020).
45. C. G. Parker et al., Ligand and Target Discovery by Fragment-Based Screening in Human Cells. Cell 168, 527-541.e₅₂₉ (2017).
46. Y. Wang et al., Expedited mapping of the ligandable proteome using fully functionalized enantiomeric probe pairs. Nat. Chem. 11, 1113-1123 (2019).
47. G. C. McAlister et al., MultiNotch MS3 Enables Accurate, Sensitive, and Multiplexed Detection of Differential Expression across Cancer Cell Line Proteomes. Anal. Chem. 86, 7150-7158 (2014).
48. G. Gonnet, M. Cohen, S. Benner, Exhaustive matching of the entire protein sequence database. Science 256, 1443-1445 (1992).
49. J. Colicelli, Human RAS Superfamily Proteins and Related GTPases. Sci. Signal. 2004, re₁₃-re₁₃ (2004).
50. R. Spencer-Smith et al., Inhibition of RAS function through targeting an allosteric regulatory site. Nat. Chem. Biol. 13, 62-68 (2017).
51. M. E. Welsch et al., Multivalent Small-Molecule Pan-RAS Inhibitors. Cell 168, 878-889.e₈₂₉ (2017).
52. T. Maurer et al., Small-molecule ligands bind to a distinct pocket in Ras and inhibit SOS-mediated nucleotide exchange activity. Proc. Natl. Acad. Sci. USA 109, 5299-5304 (2012).
53. Q. Sun et al., Discovery of Small Molecules that Bind to K-Ras and Inhibit Sos-Mediated Activation. Angew. Chem. Int. Ed. 51, 6140-6143 (2012).
54. J. M. Ostrem, K. M. Shokat, Direct small-molecule inhibitors of KRAS: from structural insights to mechanism-based design. Nat. Rev. Drug Discov. 15, 771-785 (2016).
55. J. Downward, Targeting RAS signalling pathways in cancer therapy. Nat. Rev. Cancer 3, 11-22 (2003).
56. A. G. Stephen, D. Esposito, R. K. Bagni, F. McCormick, Dragging ras back in the ring. Cancer Cell 25, 272-281 (2014).
57. B. A. Lanman et al., Discovery of a Covalent Inhibitor of KRAS(G12C) (AMG 510) for the Treatment of Solid Tumors. J. Med. Chem. 63, 52-65 (2020).
58. R. E. Kleiner, L. E. Hang, K. R. Molloy, B. T. Chait, T. M. Kapoor, A Chemical Proteomics Approach to Reveal Direct Protein-Protein Interactions in Living Cells. Cell Chemical Biology 25, 110-120.e₁₁₃ (2018).
59. D. P. Murale, S. C. Hong, M. M. Haque, J.- S. Lee, Photo-affinity labeling (PAL) in chemical proteomics: a handy tool to investigate protein-protein interactions (PPIs).Proteome Science 15, 14 (2017).
60. S. M. Margarit et al., Structural evidence for feedback activation by Ras. GTP of the Ras-specific nucleotide exchange factor SOS. Cell 112, 685-695 (2003).
61. Y. Ito et al., Regional polysterism in the GTP-bound form of the human c-Ha-Ras protein. Biochemistry 36, 9109-9119 (1997).
62. E. Joeh et al., Mapping glycan-mediated galectin-3 interactions by live cell proximity labeling. Proc. Natl. Acad. Sci. USA 117, 27329-27338 (2020).

What is claimed:

1. A macrostructure comprising an antiparallel coiled-coil, wherein the antiparallel coiled-coil comprises:
a first coil of Formula I and a second coil of Formula II:
T₁-g₀-a₁-b₁-c₁-d₁-e₁-f₁-g₁-a₂-b₂-c₂-d₂-e₂-f₂-g₂-a₃-b₃-c₃-d₃-e₃-f₃-T₂ (I)
T₃-f′₀-g′₀-a′₁-b′₁-c′₁-d′₁-e′₁-f′₁-g′₁-a′₂-b′₂-c′₂-d′₂-e′₂-f′₂-g′₂-a′₃-b′₃-c′₃-d′₃-e′₃-T₄ (II),
wherein:
each a₁₋₃, b₁₋₃, c₁₋₃, d₁₋₃, e₁₋₃, f₁₋₃, g₀₋₂, a′₁₋₃, b′₁₋₃, c′₁₋₃, d′₁₋₃, e′₁₋₃, f′₀₋₂, and g′₀₋₂ is independently absent or a residue selected from the group consisting of modified or unmodified amino acid residues and analogues thereof;
one or more of the following residue pairs are covalently bound by a linker: g₀-g′₂, g₁-g′₁, g₂-g′₀, a₁-d′₃, a₂-d′₂, a₃-d′₁, d₁-a′₃, d₂-a′₂, d₃-a′₁, e₁-e′₃, e₂-e′₂, and e₃-e′₁;
each T₁ and T₃ is independently a point of attachment from a terminal nitrogen to one or more (preferably one or two) moieties, wherein each moiety is independently H, -PG1, —C(O)R, —C(O)NR₂, —C(O)NH₂, —R, —C(O)OR, an amino acid or analogue thereof, a peptide, a targeting moiety, or a tag, where PG₁ is an amine protecting group and each R is independently hydrogen, an alkyl, an alkenyl, an alkynyl, a cycloalkyl, an aryl, a heteroaryl, a heterocyclyl, an arylalkyl, a peptide, a targeting moiety, or a tag; and
each T₂ and T₄ is independently a point of attachment from a terminal carbonyl to H, —OPG₂, —NPG₂, —OR, —OH, —NR₂, —NH₂, —N(R)C(O)C₁₋₆ alkyl, —N(H)C(O)C₁₋₆ alkyl, an amino acid or analogue thereof, a peptide, a targeting moiety, or a tag, where PG₂ is a carboxylic acid protecting group and each R is independently hydrogen, an alkyl, an alkenyl, an alkynyl, a cycloalkyl, an aryl, a heteroaryl, a heterocyclyl, an arylalkyl, a peptide, a targeting moiety, or a tag;
and wherein:
the first coil comprises at least ten contiguous residues, wherein the at least ten contiguous residues have the formula X₁-X₂-X₃-X₄-X₅-X₆-X₇-X₈-X₉-X₁₀-X₁₁-X₁₂-X₁₃-X₁₄-X₁₅-X_{16;}
the second coil comprises at least ten contiguous residues, wherein the at least ten contiguous residues have the formula X′₁-X′₂-X′₃-X′₄-X′₅-X′₆-X′₇-X′₈-X′₉-X′₁₀-X′₁₁-X′₁₂-X′₁₃-X′₁₄-X′₁₅-X′₁₆; and
wherein each residue is selected from the groups indicated below (superscript letters indicate each residue's location within Formula I and Formula II; residues in the a, a′, d, d′, e, e′, g, and g′ positions can optionally be modified to facilitate attachment of a linker or replaced with a linker; underlined residues are particularly preferred)

| First Coil | | | Second Coil | | |
|---|---|---|---|---|---|
| | | Preferred | | | Preferred |
| Residue | Group | Residue(s) | Residue | Group | Residue(s) |
| ^{g}X₁ | Any residue | Phe, Trp | | | |
| ^{a}X₂ | Any | Cys, HCys, Leu, | ^{d}X′₁ | Any | Cys, HCys, Leu, |
| | hydrophobic | Ile, allylleucine, | | hydrophobic | Ile, allylleucine, |
| | residue | Val, allylglycine, | | residue | Val, allylglycine, |
| | | Thr, | | | Thr, |
| | | selenocysteine, | | | selenocysteine, |
| | | hexafluoroleucine, | | | hexafluoroleucine, |
| | | hexafluorovaline | | | hexafluorovaline |
| | | (or analogue of | | | (or analogue of |
| | | any of the | | | any of the |
| | | preceding | | | preceding |
| | | residues) | | | residues) |
| ^{b}X₃ | Any residue | Gly | ^{e}X′₂ | Any residue | Ala |
| ^{c}X₄ | Any residue | Arg | ^{f}X′₃ | Any residue | Trp |
| ^{d}X₅ | Any | Cys, HCys, Leu, | ^{g}X′₄ | Any residue | Arg |
| | hydrophobic | Ile, allylleucine, | | | |
| | residue | Val, allylglycine, | | | |
| | | Thr, | | | |
| | | selenocysteine, | | | |
| | | hexafluoroleucine, | | | |
| | | hexafluorovaline | | | |
| | | (or analogue of | | | |
| | | any of the | | | |
| | | preceding | | | |
| | | residues) | | | |
| ^{e}X₆ | Any residue | Cys | ^{a}X′₅ | Any | Cys, HCys, Leu, |
| | | | | hydrophobic | Ile, allylleucine, |
| | | | | residue | Val, allylglycine, |
| | | | | | Thr, |
| | | | | | selenocysteine, |
| | | | | | hexafluoroleucine, |
| | | | | | hexafluorovaline |
| | | | | | (or analogue of |
| | | | | | any of the |
| | | | | | preceding |
| | | | | | residues) |
| ^{f}X₇ | Any residue | Thr | ^{b}X′₆ | Any residue | Arg |
| ^{g}X₈ | Any residue | Glu, Carboxylic | ^{c}X′₇ | Any residue | Glu |
| | | Acid Isostere | | | |
| ^{a}X₉ | Any | Cys, HCys, Leu, | ^{d}X′₈ | Any | Cys, HCys, Leu, |
| | hydrophobic | Ile, allylleucine, | | hydrophobic | Ile, allylleucine, |
| | residue | Val, allylglycine, | | residue | Val, allylglycine, |
| | | Thr, | | | Thr, |
| | | selenocysteine, | | | selenocysteine, |
| | | hexafluoroleucine, | | | hexafluoroleucine, |
| | | hexafluorovaline | | | hexafluorovaline |
| | | (or analogue of | | | (or analogue of |
| | | any of the | | | any of the |
| | | preceding | | | preceding |
| | | residues) | | | residues) |
| ^{b}X₁₀ | Any residue | Leu, L- | ^{e}X′₉ | Any residue | Glu |
| | | homoarginine | | | |
| ^{c}X₁₁ | Any residue | Lys, Arg | ^{f}X′₁₀ | Any residue | Arg |
| ^{d}X₁₂ | any | Cys, HCys, Leu, | ^{g}X′₁₁ | Any residue | Glu |
| | hydrophobic | Ile, allylleucine, | | | |
| | residue | Val, allylglycine, | | | |
| | | Thr, | | | |
| | | selenocysteine, | | | |
| | | hexafluoroleucine, | | | |
| | | hexafluorovaline | | | |
| | | (or analogue of | | | |
| | | any of the | | | |
| | | preceding | | | |
| | | residues) | | | |
| ^{e}X₁₃ | Any residue | Arg | ^{a}X′₁₂ | Any | Cys, HCys, Leu, |
| | | | | hydrophobic | Ile, allylleucine, |
| | | | | residue | Val, allylglycine, |
| | | | | | Thr, |
| | | | | | selenocysteine, |
| | | | | | hexafluoroleucine, |
| | | | | | hexafluorovaline |
| | | | | | (or analogue of |
| | | | | | any of the |
| | | | | | preceding |
| | | | | | residues) |
| ^{f}X₁₄ | Any residue | Glu, Asn, Amide | ^{b}X′ ₁₃ | Any residue | Ala |
| | | isostere, | | | |
| | | Carboxylic acid | | | |
| | | isostere | | | |
| ^{g}X₁₅ | Any residue | Gly | ^{c}X′₁₄ | Any residue | Arg |
| ^{a}X₁₆ | Any residue | Asn, Amide | ^{d}X′₁₅ | Any | Cys, HCys, Leu, |
| | | isostere | | hydrophobic | Ile, allylleucine, |
| | | | | residue | Val, allylglycine, |
| | | | | | Thr, |
| | | | | | selenocysteine, |
| | | | | | hexafluoroleucine, |
| | | | | | hexafluorovaline |
| | | | | | (or analogue of |
| | | | | | any of the |
| | | | | | preceding |
| | | | | | residues) |
| | | | ^{e}X′₁₆ | Any residue | Cys |

2. The macrostructure of claim 1, wherein:
the length of any linker between residue pairs g₀-g′₂, g₁-g′₁, g₂-g′₀, e₁-e′₃, e₂-e′₂, and e₃-e′₁ is such that the spatial distance between the Cα positions of each residue in the pair is 10-25 Å; and
the length of any linker between residue pairs a₁-d′₃, a₂-d′₂, a₃-d′₁, d₁-a′₃, d₂-a′₂, and d₃-a′₁ is such that the spatial distance between the Cα positions of each residue in the pair is 5-15 Å.

3. The macrostructure of claim 1 or claim 2, wherein at least g₀, a₁, b₁, c₁, d₁, e₁, f₁, g₁, a₂, b₂, c₂, d₂, e₂, f₂, g₂, and a₃, are present in the first coil and at least d′₁, e′₁, f′₁, g′₁, a′₂, b′₂, c′₂, d′₂, e′₂, f₂, g′₂, a′₃, b′₃, c′₃, d′₃, and e′₃ are present in the second coil.

4. The macrostructure of any one of claims 1-3, wherein each residue independently has the formula 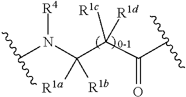
wherein:
R^{1a}, R^{1b}, R^{1c}, and R^{1d} are each independently hydrogen, an amino acid side chain, an alkyl, an alkenyl, an alkynyl, a cycloalkyl, a heterocyclyl, an aryl, a heteroaryl, or an arylalkyl, wherein each amino acid side chain, alkyl, alkenyl, alkynyl, cycloalkyl, heterocyclyl, aryl, heteroaryl, and arylalkyl can be optionally substituted with H, an alkyl, an alkenyl, an alkynyl, an azide, —OR⁵, or —SR⁵; and wherein when a linker covalently binds to a residue, the linker is attached to or replaces one of R^{1a}, R^{1b}, R^{1c}, and R^{1d};
each R⁴ is independently hydrogen, an alkyl, an alkenyl, an alkynyl, a cycloalkyl, a heterocyclyl, an aryl, a heteroaryl, or an arylalkyl; and
each R⁵ is independently selected from the group consisting of H, -PG (where PG is a protecting group), an alkyl, an alkenyl, an alkynyl, a cycloalkyl, an aryl, a heteroaryl, a heterocyclyl, and an arylalkyl.

5. The macrostructure of any one of claims 1-4, wherein a linker between at least one of residue pairs g₀-g′₂, g₁-g′₁, g₂-g′₀, e₁-e′₃, e₂-e′₂, and e₃-e′₁ is present.

6. The macrostructure of any one of claims 1-5, wherein a linker between at least one of residue pairs g₀-g′₂, g₁-g′₁, g₂-g′₀, e₁-e′₃, e₂-e′₂, and e₃-e′₁ has the formula —Zₙ—, wherein n is a number from 1 to 25 and each Z is independently selected at each occurrence thereof from the group consisting of alkylene, alkenylene, arylene, heteroarylene, triazole-diyl, thiazole-diyl, oxazole-diyl, ethers, amides, esters, maleimides, thioethers, O, S, and Se.

7. The macrostructure of any one of claims 1-6, wherein a linker between at least one of residue pairs g₀-g′₂, g₁-g′₁, g₂-g′₀, e₁-e′₃, e₂-e′₂, and e₃-e′₁ has a formula selected from (a) the group consisting of: 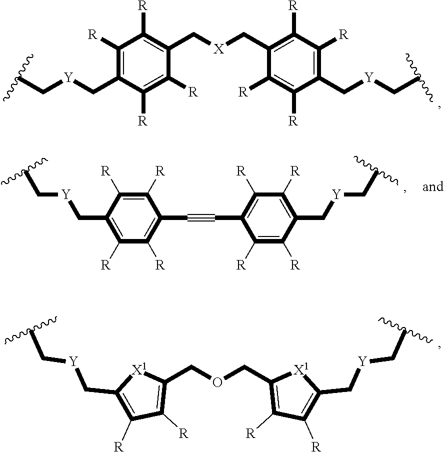
(b) the group consisting of: 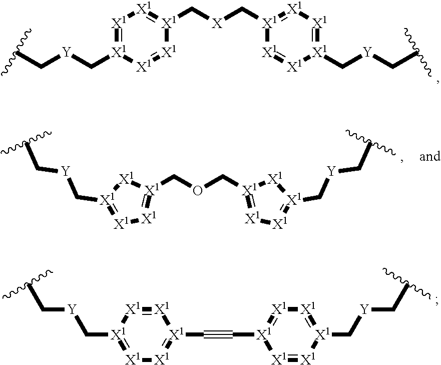
wherein:
X in group (a) and group (b) is O, S, CR₂, NR, or P (preferably O, S, CH₂ or NR);
each X₁ in group (a) is independently O, S, NH, or NR;
each X₁ in group (b) is independently O, S, C, CR, N, NH, or NR;
each R in group (a) and group (b) is independently H, alkyl, or aryl; and
each Y in group (a) and group (b) is S.

8. The macrostructure of any one of claims 1-7, wherein a linker between at least one of residue pairs g₀-g′₂, g₁-g′₁, g₂-g′₀, e₁-e′₃, e₂-e′₂, and e₃-e′₁ is selected from the group consisting of

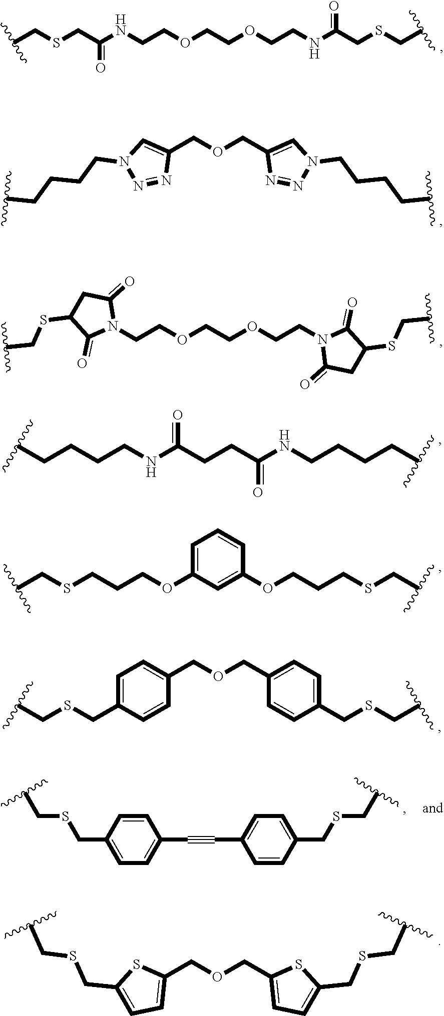

9. The macrostructure of any one of claims 1-8, wherein a linker between at least one of residue pairs a₁-d′₃, a₂-d′₂, a₃-d′₁, d₁-a′₃, d₂-a′₂, and d₃-a′₁ is present.

10. The macrostructure of any one of claims 1-9, wherein a linker between at least one of residue pairs a₁-d′₃, a₂-d′₂, a₃-d′₁, d₁-a′₃, d₂-a′₂, and d₃-a′₁ is selected from the group consisting of disulfides, diselenides, C₁₋₈ alkylene, C₂₋₈ alkenylene, arylene, heteroarylene, triazole-diyl, and thiazole-diyl.

11. The macrostructure of any one of claims 1-10, wherein a linker between at least one of residue pairs a₁-d′₃, a₂-d′₂, a₃-d′₁, d₁-a′₃, d₂-a′₂, and d₃-a′₁ is a disulfide bond from a cysteine or homocysteine residue, a diselenide from a selenocysteine residue, an alkylene from an allylglycine residue, or an arylene linker.

12. The macrostructure of any one of claims 1-11, wherein the antiparallel coiled-coil is of Formula III: 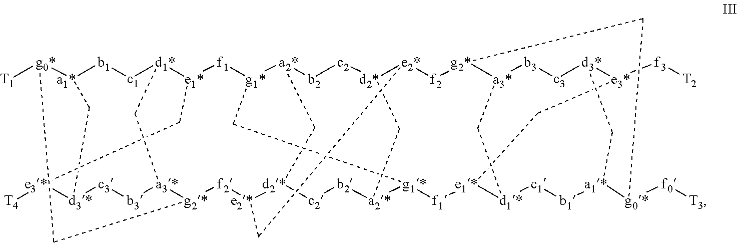
wherein:
each dotted line represents, independently, an optional linker and each residue independently has the formula 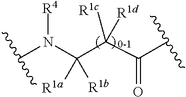
wherein:
R^{1a}, R^{1b}, R^{1c}, and R^{1d} are each independently hydrogen, an amino acid side chain, an alkyl, an alkenyl, an alkynyl, a cycloalkyl, a heterocyclyl, an aryl, a heteroaryl, or an arylalkyl, wherein each amino acid side chain, alkyl, alkenyl, alkynyl, cycloalkyl, heterocyclyl, aryl, heteroaryl, and arylalkyl can be optionally substituted with H, an alkyl, an alkenyl, an alkynyl, an azide, —OR⁵, or —SR⁵; and wherein when a linker covalently binds to a residue, the linker is attached to or replaces one of R^{1a}, R^{1b}, R^{1c}, and R^{1d};
each R⁴ is independently hydrogen, an alkyl, an alkenyl, an alkynyl, a cycloalkyl, a heterocyclyl, an aryl, a heteroaryl, or an arylalkyl; and
each R⁵ is independently selected from the group consisting of H, -PG (where PG is a protecting group), an alkyl, an alkenyl, an alkynyl, a cycloalkyl, an aryl, a heteroaryl, a heterocyclyl, and an arylalkyl.

13. The macrostructure of claim 12, wherein at least one of the following conditions is met:
(A) in at least one a, a′, d, or d′ residue, (i) one of R^{1a} and R^{1c} is the side chain of a modified or unmodified amino acid selected from the group consisting of cysteine, homocysteine, selenocysteine, leucine, isoleucine, hexafluoroleucine, valine, hexafluorovaline, allylglycine, threonine, and analogues of each of the preceding residues, and (ii) R^{1b}, R^{1d}, and the other of R^{1a} and R^{1c} are each independently hydrogen, a C₁₋₃ alkyl, or a C₂₋₃ alkenyl;
(B) in at least one e, e′, g, or g′ residue, (i) one of R^{1a} and R^{1c} is an amino acid side chain and (ii) R^{1b}, R^{1d}, and the other of R^{1a} and R^{1c} are each independently hydrogen or a C₁₋₃ alkyl.

14. The macrostructure of any one of claims 1-13, wherein the antiparallel coiled-coil has the formula: [symbol]
(two-dimensional view)
wherein each dotted line is independently an optional linker.

15. The macrostructure of any one of claims 1-14, wherein the macrostructure is CHD-1, CHD-2, CHD-3, CHD-4, or CHD-5.

16. A pharmaceutical composition comprising a macrostructure according to any one of claims 1-15 and a pharmaceutically acceptable vehicle.

17. A method of inhibiting Ras signaling in a cell, said method comprising:
contacting the cell with a macrostructure according to any one of claims 1-15 under conditions effective to inhibit Ras signaling in the cell.

18. The method of claim 17, wherein the cell is a mammalian cell.

19. The method of claim 17 or claim 18, wherein the cell expresses a mutated Ras protein.

20. The method of any one of claims 17-19, wherein said contacting is carried out in a subject.

21. A method of treating in a subject a disorder mediated by Ras signaling, said method comprising:
administering to the subject a macrostructure according to any one of claims 1-15 or a pharmaceutical formulation according to claim 16 under conditions effective to treat the disorder in the subject.

22. A method of treating a cellular proliferative disorder, differentiative disorder, and/or neoplastic condition in a subject in need thereof, the method comprising:
administering to the subject a macrostructure according to any one of claims 1-15 or a pharmaceutical formulation according to claim 16 under conditions effective to treat the cellular proliferative disorder, differentiative disorder, and/or neoplastic condition in the subject.

23. The method according to claim 22, wherein the cellular proliferative disorder, differentiative disorder, and/or neoplastic condition is selected from the group consisting of fibrosarcoma, myosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, gastric cancer, esophageal cancer, rectal cancer, pancreatic cancer, ovarian cancer, prostate cancer, uterine cancer, cancer of the head and neck, skin cancer, brain cancer, squamous cell carcinoma, sebaceous gland carcinoma, papillary carcinoma, papillary adenocarcinoma, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular cancer, small cell lung carcinoma, non-small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma, leukemia, lymphoma, and Kaposi sarcoma; hematopoietic neoplastic disorders; cellular proliferative and/or differentiative disorders of the breast; cellular proliferative and/or differentiative disorders of the lung; cellular proliferative and/or differentiative disorders of the colon; cellular proliferative and/or differentiative disorders of the liver; cellular proliferative and/or differentiative disorders of the ovary; a cancer mediated by a mutated Ras protein; and immunoproliferative disorders.

24. The method according to any one of claims 21-23, wherein the disorder and/or condition is pancreatic ductal adenocarcinoma, rectal adenocarcinoma, plasma cell myeloma, colon adenocarcinoma, bile duct carcinoma, chronic myelomonocytic leukemia, acute myeloid leukemia, melanoma, lung adenocarcinoma, rhabdomyosarcoma, endometrium carcinoma, salivary gland carcinoma, thyroid carcinoma, bladder carcinoma, mouth carcinoma, or ovarian carcinoma.

25. The method of any one of claims 20-24, wherein the subject is a mammal.

26. The method of any one of claims 20-25, wherein the subject is a primate (e.g., human).

27. The method of any one of claims 21-26, wherein the disorder or condition is mediated by a mutated Ras protein.

28. The method according to claim 19 or claim 27, wherein the mutated Ras protein is an H-Ras isoform.

29. The method according to claim 19 or claim 27, wherein the mutated Ras protein is a K-Ras isoform.

30. The method according to any one of claims 19 and 27-29, wherein the mutated Ras protein has one or more mutations selected from the group consisting of G12C, G12D, G12S, G12V, G13D, G12A, G12C, G12D, G12R, G12S, G12V, G13A, G13C, G13R, G13S, G13D, Q61E, Q61H, Q61L, Q61K, Q61P, and Q61R.
